(19) United States
(12) Patent Application Publication
(10) Pub. No.: US 2024/0374579 A1
(43) Pub. Date: Nov. 14, 2024

(54) BICYCLIC PGDH INHIBITORS AND METHODS OF MAKING AND USING

(71) Applicant: EPIRIUM BIO INC., San Diego, CA (US)

(72) Inventors: Sundeep DUGAR, San Jose, CA (US); Bruce FAHR, San Diego, CA (US); Roopa RAI, San Carlos, CA (US); Michael J. GREEN, Half Moon Bay, CA (US)

(21) Appl. No.: 18/292,890

(22) PCT Filed: Jul. 27, 2022
(86) PCT No.: PCT/US2022/038515

§ 371 Date: Jan. 26, 2024

Related U.S. Application Data
(60) Provisional application No. 63/226,671, filed on Jul. 28, 2021.
(60) Provisional application No. 63/226,273, filed on Jul. 28, 2021.

(51) Int. Cl. A61K 31/4545; A61K 31/454; A61K 31/5377; A61K 31/541; C07D 401/06; C07D 401/14; C07D 471/04

(52) U.S. Cl. CPC A61K 31/4545; A61K 31/454; A61K 31/5377; A61K 31/541; C07D 401/06; C07D 401/14; C07D 471/04

(57) ABSTRACT

Disclosed herein are 15-hydroxyprostaglandin dehydrogenase inhibitor compounds. Such compounds may be administered to subjects that may benefit from modulation of prostaglandin levels.

### CROSS REFERENCE TO RELATED APPLICATIONS

This application claims the benefit of U.S. Provisional Application No. 63/226,671, filed Jul. 28, 2021, and U.S. Provisional Application No. 63/226,673, filed Jul. 28, 2021, which are hereby incorporated by reference in their entirety.

### BACKGROUND OF THE INVENTION

Prostaglandins are a group of physiologically active lipid compounds with diverse biological effects including vasodilation, inhibition of platelet aggregation, bronchodilation, bronchoconstriction, immune responses, contraction and relaxation of gastrointestinal smooth muscles, gastric acid secretion, gastric mucus secretion, uterus contraction, lipolysis inhibition, neurotransmission, clotting, hyperalgesia, and pyrexia.

Treatment of diseases or disorders may require activation of prostaglandins, or inhibition of inactivation of prostaglandins. Hydroxyprostaglandin dehydrogenases, such as 15-hydroxyprostaglandin dehydrogenase (15-PGDH) are involved in the inactivation of prostaglandins. As such, diseases/disorders associated with prostaglandins can be prevented, treated and/or managed using inhibitors of hydroxyprostaglandin dehydrogenase such as inhibitors of 15-PGDH.

### SUMMARY OF THE INVENTION

In one aspect, provided herein is a PGDH inhibitor having the structure of Formula (V), or a pharmaceutically acceptable salt or solvate thereof:

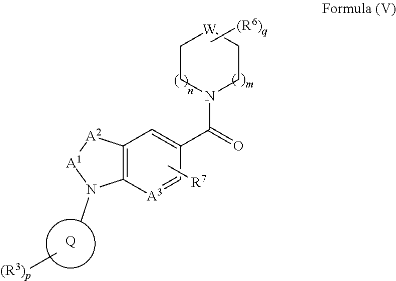

wherein,
ring Q is C₆ aryl or 5 to 10-membered heteroaryl;
A¹ is CR¹ or N and A² is CR² or N, provided that at least one of A¹ or A² is N;
A³ is N or CR⁷;
W is —CR⁶R⁶—, —O—, —S—, —NR⁵—, —S(O)—, —S(O)₂—, or —C(O)—;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁵ is H or C₁-C₆ alkyl; and
each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
or two R⁶ can join together with the atom(s) to which they are attached to form a C₃-C₆ cycloalkyl or C₃-C₈ heterocycloalkyl ring;
R⁷ is H, halogen, —OR¹⁰, —C(O)R¹⁰, —C(O)OR¹⁰, —CN, —C(O)NR⁸R⁹, —NR⁸C(O)R⁹, substituted or unsubstituted C₁-C₆ alkyl, or substituted or unsubstituted C₃-C₈ cycloalkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
n and m are each independently 0, 1, 2, or 3;
q is 0, 1, or 2; and
p is 1, 2, or 3.

In some embodiments, ring Q is a bicyclic or monocyclic heteroaryl comprising 1, 2, or 3 heteroatoms selected from O, S, or N. In some embodiments, wherein ring Q is a phenyl, pyrimidinyl, or pyridinyl.

In some embodiments, the compound has the structure of Formula (VI), or a pharmaceutically acceptable salt or solvate thereof:

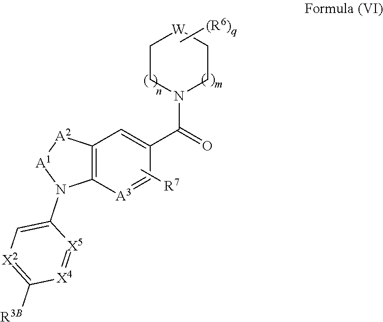

wherein,
X² is N, NR^{3A}, or CR^{3A};
X³ is N or CR^{3B};
X⁴ is N, NR^{3C}, or CR^{3C}; and
R^{3A}, R^{3B}, and R^{3C} are each independently H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R^{3A} and R^{3B} together with the atoms to which they are attached form a substituted or unsubstituted 5 to 6-membered aryl or heteroaryl; or
R^{3B} and R^{3C} together with the atoms to which they are attached form a substituted or unsubstituted 5 to 6-membered aryl or heteroaryl; and
wherein CR^{3A}, CR^{3B}, and CR^{3C} are not all H at the same time.

In some embodiments, the compound has the structure of Formula (VIa), (VIb), or (VIc), or a pharmaceutically acceptable salt or solvate thereof:

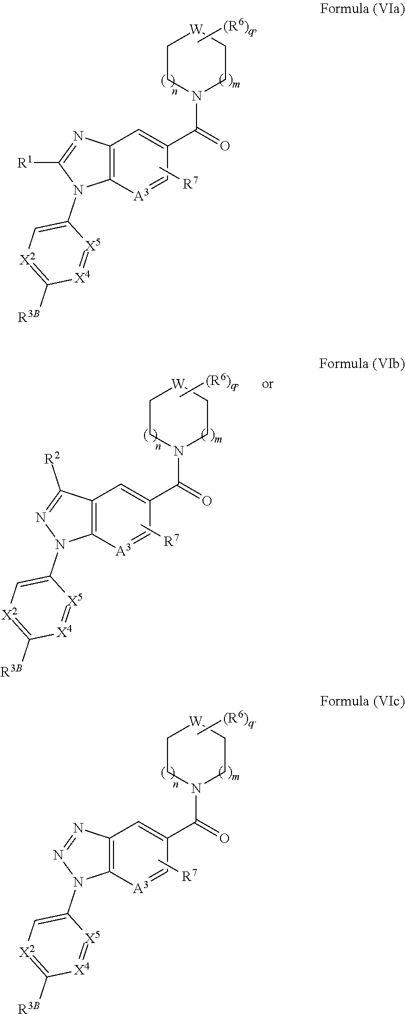

In some embodiments, the compound has the structure of Formula (VII), or a pharmaceutically acceptable salt or solvate thereof:

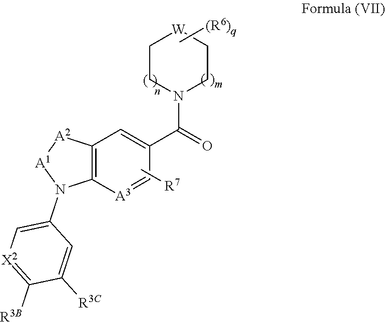

wherein,
X² is N or CH; and
R^{3C} is H or halogen and R^{3B} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl; or
R^{3B} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

In some embodiments, the compound has the structure of Formula (VIIa), (VIIb), or (VIIc), or a pharmaceutically acceptable salt or solvate thereof:

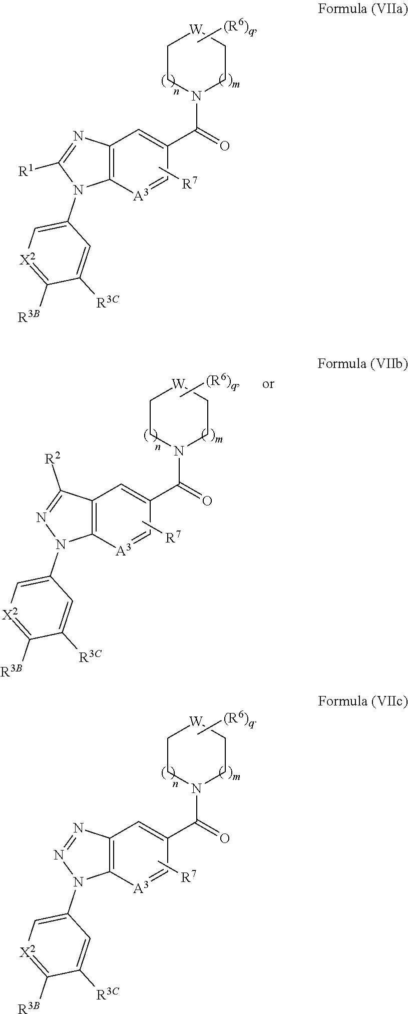

In some embodiments, Q is a 5-membered heteroaryl selected from triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, and tetrazolyl.

In some embodiments, wherein ring Q is

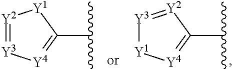

wherein Y¹ is O, S, or NR^{3D}; Y² is N or CR^{3A}; Y³ and Y⁴ are each independently N or CR^{3B}; R^{3A} and R^{3B} are each independently selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

In some embodiments, wherein the compound has the structure of Formula (VIIIa), (VIIIb), (VIIIc), (VIIId), (VIIIe), or (VIIIf), or a pharmaceutically acceptable salt or solvate thereof:

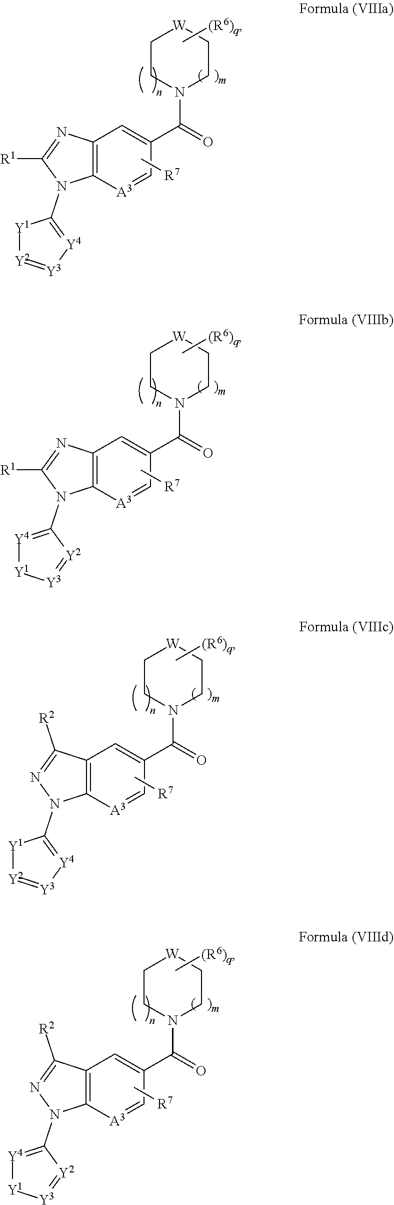

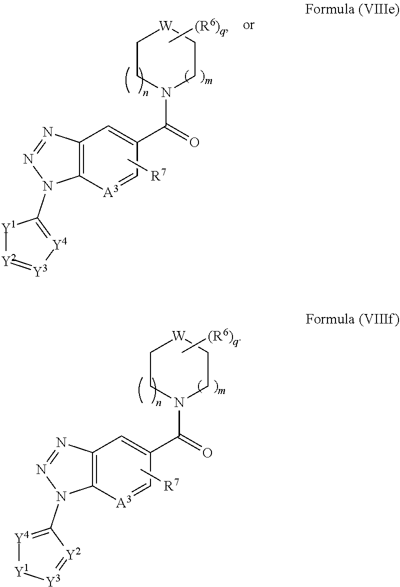

In another aspect, provided herein is a PGDH inhibitor having the structure of Formula (I), or a pharmaceutically acceptable salt or solvate thereof:

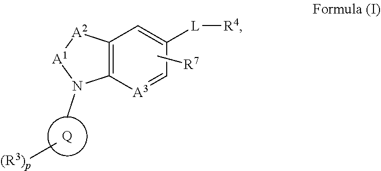

wherein,
ring Q is C₆ aryl or 5- to 10-membered heteroaryl;
L is —CR^{13A}R^{13B}, —C(O)—, —S—, —S(O)—, —S(O)₂—, or —S(O₂)NH—;
A¹ is N or CR¹ and A² is N or CR², provided that at least one of A¹ or A² is N;
A³ is N or CR⁷;
R¹ and R² are each independently H, halogen, —CN, —OR¹⁰, —C(O)R¹⁰, —C(O)OR¹⁰, —NR⁸R⁹, —C(O)NR⁸R⁹, —NR⁸C(O)R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —NR¹²C(O)OR⁹, —SOR¹¹, —SO₂R¹¹, —SO₂NR⁸R⁹, —NR¹²C(O)R¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁴ is substituted or unsubstituted C₁-C₈ alkyl, substituted or unsubstituted C₂-C₈ alkenyl, substituted or unsubstituted C₁-C₈ heteroalkyl;
or R⁴ is

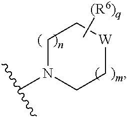

wherein
   W is —CR⁶R⁶—, —O—, —S—, —NR⁵—, —S(O)₂—, or —C(O)—;
   R⁵ is H or C₁-C₆ alkyl;
   each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, —NR⁸C(O)R⁹, —SR⁸, substituted or unsubstituted C₁-C₆ alkyl, or substituted or unsubstituted C₃-C₈ cycloalkyl;
   or two R⁶ can join together with the atom(s) to which they are attached to form a C₃-C₆ cycloalkyl or C₃-C₈ heterocycloalkyl ring;
   n and m are each independently 0, 1, 2, or 3; and
   q is 0, 1, 2, 3, 4, 5, or 6;
R⁷ is H, halogen, —OR¹⁰, —C(O)R¹⁰, —C(O)OR¹⁰, —CN, —C(O)NR⁸R⁹, —NR⁸C(O)R⁹, substituted or unsubstituted C₁-C₆ alkyl, or substituted or unsubstituted C₃-C₈ cycloalkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, and C₃-C₁₀ heterocycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
R^{13A} and R^{13B} are each independently H, CF₃, halogen, or C₁-C₆ alkyl; and
p is 1, 2, 3, or 4.

In some embodiments, the compound has the structure of Formula (IIa), (IIb), or (IIc), or a pharmaceutically acceptable salt or solvate thereof:

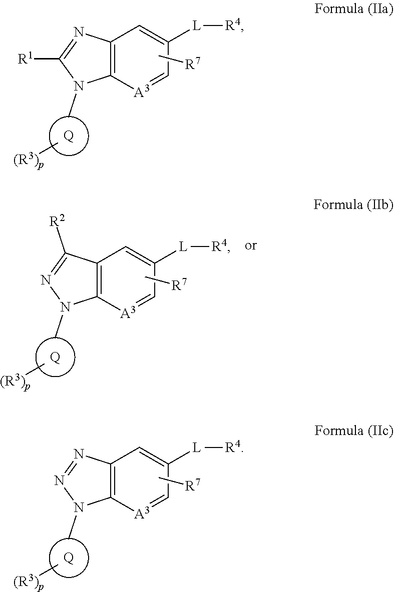

In some embodiments, the compound is selected from Table 1a or Table 1b, or a pharmaceutically acceptable salt or solvate thereof.

In some embodiments, is a pharmaceutical composition comprising a compound described herein, or a pharmaceutically acceptable salt or solvate thereof; and a pharmaceutically acceptable excipient.

In another aspect, provided herein is a method of promoting and/or stimulation skin pigmentation, comprising administering one or more of the compositions described herein to a subject in need thereof.

In another aspect, provided herein is a method of inhibiting hair loss, comprising administering one or more of the compositions described herein to a subject in need thereof.

method of preventing and/or treating skin inflammation and/or damage, comprising administering one or more of the compositions described herein to a subject in need thereof.

In another aspect, provided herein is a method of preventing and/or treating vascular insufficiency, comprising administering one or more of the compositions described herein to a subject in need thereof.

In another aspect, provided herein is a method of preventing, treating, minimizing and/or reversing congestive heart failure, cardiomyopathy, comprising administering one or more of the compositions described herein to a subject in need thereof.

In another aspect, provided herein is a method of reducing cardiac ejection fraction, comprising administering one or more of the compositions described herein to a subject in need thereof.

In another aspect, provided herein is a method of preventing and/or treating a gastrointestinal disease, comprising administering one or more of the compositions described herein to a subject in need thereof.

In another aspect, provided herein is a method of preventing and/or treating renal dysfunction, comprising administering one or more of the compositions described herein to a subject in need thereof.

In another aspect, provided herein is a method of stimulation bone resorption and bone formation, comprising administering one or more of the compositions described herein to a subject in need thereof.

In another aspect, provided herein is a method of stimulating tissue regeneration by stimulating, comprising administering one or more of the compositions described herein to a subject in need thereof.

In another aspect, provided herein is a method of modulating cervical ripening, comprising administering one or more of the compositions described herein to a subject in need thereof.

In another aspect, provided herein is a method of promoting neuroprotection and/or stimulating neuronal regeneration, comprising administering one or more of the compositions described herein to a subject in need thereof.

In another aspect, provided herein is a method of treating and/or preventing a neurological disorder, a neuropsychiatric disorder, a neural injury, a neural toxicity disorder, a neuropathic pain, or a neural degenerative disorder, comprising administering one or more of the compositions described herein to a subject in need thereof.

In another aspect, provided herein is a method of treating and/or preventing fibrotic or adhesion disease, disorder or condition, comprising administering one or more of the compositions described herein to a subject in need thereof.

In another aspect, provided herein is a method of reducing and/or preventing scar formation, comprising administering one or more of the compositions described herein to a subject in need thereof.

In another aspect, provided herein is a method of treating and/or preventing muscle disorder, muscle injury and/or muscle atrophy, comprising administering one or more of the compositions described herein to a subject in need thereof.

In another aspect, provided herein is a method of treating and/or preventing fibrosis, comprising administering one or more of the compositions described herein to a subject in need thereof.

In another aspect, provided herein is a method of treating and/or preventing idiopathic pulmonary fibrosis, comprising administering one or more of the compositions described herein to a subject in need thereof.

In another aspect, provided herein is a method of treating and/or preventing kidney fibrosis, comprising administering one or more of the compositions described herein to a subject in need thereof.

In another aspect, provided herein is a method of stimulating muscle regeneration, comprising administering one or more of said compositions described herein to a subject in need thereof.

In another aspect, provided herein is a method of promoting organ fitness, comprising administering one or more of said compositions described herein to a subject in need thereof.

In another aspect, provided herein is a method of promoting wound healing, comprising administering one or more of said compositions described herein to a subject in need thereof.

In another aspect, provided herein is a method of treating acute kidney injury, comprising administering one or more of said compositions described herein to a subject in need thereof.

In another aspect, provided herein is a method of treating sarcopenia, comprising administering one or more of said compositions described herein to a subject in need thereof.

In another aspect, provided herein is a method of treating a neuromuscular disease, comprising administering one or more of said compositions of any of the preceding claims to a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₆alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂.

“Alkoxy” refers to a radical of the formula —OR^{a} where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂.

“Carbocycle” refers to a saturated, unsaturated or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₈ fully saturated cycloalkyl or C₃-C₈ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₇ heterocycloalkenyl), from two to five carbon atoms (e.g., C₂-C₈ fully saturated heterocycloalkyl or C₂-C₈ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂.

“Heteroaryl” or “aromatic heterocycle” refers to a radical derived from a heteroaromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, thiophene, benzthiazole, and imdazopyridine. An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

The term “substituted” refers to moieties having substituents replacing a hydrogen on one or more carbons or substitutable heteroatoms, e.g., Nil, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR a, —R—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)OR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR a, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)—. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that may induce a particular response in target cells, e.g., reduction of platelet adhesion and/or cell migration. The specific dose may vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

The terms “antagonist” and “inhibitor” are used interchangeably, and they refer to a compound having the ability to inhibit a biological function (e.g., activity, expression, binding, protein-protein interaction) of a target protein or enzyme. Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

Whenever a protein is referred to herein, it will be understood that a single protein can be referred to by different names. For example, “15-PGDH”, “PGDH”, and “hPGDH” all refer to the same protein, 15-hydroxyprostaglandin dehydrogenase.

### Compounds

Provided herein are compounds and methods for inhibiting 15-hydroxyprostaglandin dehydrogenase (15-PGDH).

In one aspect, provided herein is a compound having the structure of Formula (I), or a pharmaceutically acceptable salt or solvate thereof:

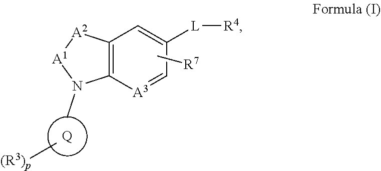

wherein,
ring Q is C₆ aryl or 5- to 10-membered heteroaryl;
L is —CR^{13A}R^{13B}, —C(O)—, —S—, —S(O)—, —S(O)₂—, or —S(O₂)NH—;
A¹ is N or CR¹ and A² is N or CR², provided that at least one of A¹ or A² is N;
A³ is N or CR⁷;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl, wherein each or which is substituted or unsubstituted with one, two, or three R¹⁴;
R⁴ is substituted or unsubstituted C₁-C₈ alkyl, substituted or unsubstituted C₂-C₈ alkenyl, substituted or unsubstituted C₁-C₈ heteroalkyl, or substituted or unsubstituted C₁-C₈ heteroalkyl, wherein each is substituted or unsubstituted with one, two, or three R¹⁴;
or R⁴ is

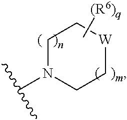

wherein
   W is —CR⁶R⁶—, —O—, —S—, —NR⁵—, —S(O)₂—, or —C(O)—;
   R⁵ is H or substituted or unsubstituted C₁-C₆ alkyl, substituted or unsubstituted cycloalkyl, or substituted or unsubstituted heterocycloalkyl;
   each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, —NR⁸C(O)R⁹, —SR⁸, substituted or unsubstituted C₁-C₆ alkyl, or substituted or unsubstituted C₃-C₈ cycloalkyl;
   or two R⁶ can join together with the atom(s) to which they are attached to form a C₃-C₆ cycloalkyl or C₃-C₈ heterocycloalkyl ring;
   n and m are each independently 0, 1, 2, or 3; and
   q is 0, 1, 2, 3, 4, 5, or 6;
R⁷ is H, halogen, —OR¹⁰, —C(O)R¹⁰, —C(O)OR¹⁰, or substituted or unsubstituted C₁-C₆ alkyl, or substituted or unsubstituted C₃-C₈ cycloalkyl, wherein the alkyl or heteroalkyl is substituted or unsubstituted with one, two, or three R¹⁴;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, or C₃-C₁₀ heterocycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
R^{13A} and R^{13B} are each independently H, CF₃, halogen, or C₁-C₆ alkyl;
each R¹⁴ is independently selected from halogen, —CN, —NR⁸R⁹, —OR¹⁰, —C(O)R¹⁰, —C(O)OR¹⁰, —C(O)NR⁸R⁹, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, or C₃-C₁₀ heterocycloalkyl; and
p is 1, 2, 3, or 4.

In some embodiments, A¹ is CR¹; and A² is N; In some embodiments, A¹ is N; and A² is CR². In some embodiments, A¹ is CH; and A² is N; In some embodiments, A¹ is N; and A² is CH;

In some embodiments, the compound of Formula (I) is a compound having the structure of Formula (IIa), or a pharmaceutically acceptable salt or solvate thereof:

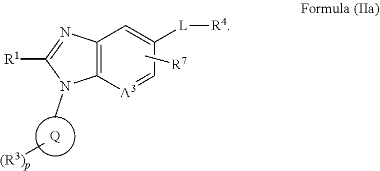

In some embodiments, the compound of Formula (I) is a compound having the structure of Formula (IIb), or a pharmaceutically acceptable salt or solvate thereof:

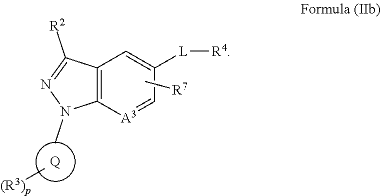

In some embodiments, the compound of Formula (I) is a compound having the structure of Formula (IIc), or a pharmaceutically acceptable salt or solvate thereof:

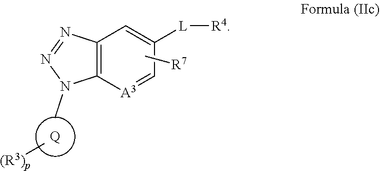

In some embodiments, A³ is CR⁷. In some embodiments, A³ is CH. In some embodiments, A³ is N.

In some embodiments, R is H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl. In some embodiments, R¹ is H, halogen, —CN, —OR¹⁰, or —NR⁸R⁹. In some embodiments, R¹ is —C(O)R¹⁰, —C(O)OR¹⁰, or —C(O)NR⁸R⁹. In some embodiments, R¹ is substituted or unsubstituted C₁-C₆ alkyl, or substituted or unsubstituted C₃-C₈ cycloalkyl. In some embodiments, R¹ is H.

In some embodiments, R² is H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl. In some embodiments, R² is H, halogen, —CN, —OR¹⁰, or —NR⁸R⁹. In some embodiments, R² is —C(O)R¹⁰, —C(O)OR¹⁰, or —C(O)NR⁸R⁹. In some embodiments, R² is substituted or unsubstituted C₁-C₆ alkyl, or substituted or unsubstituted C₃-C₈ cycloalkyl. In some embodiments, R² is H.

In another aspect, provided herein is a compound having the structure of Formula (Ie), or a pharmaceutically acceptable salt or solvate thereof:

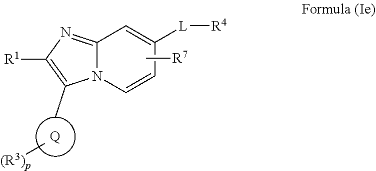

wherein,
ring Q is C₆ aryl or 5- to 10-membered heteroaryl;
L is —CR^{13A}R^{13B}, —C(O)—, —S—, —S(O)—, —S(O)₂—, or —S(O₂)NH—;
R¹ and R² are each independently H, halogen, —CN, —OR¹⁰, —C(O)R¹⁰, —C(O)OR¹⁰, —NR⁸R⁹, —NR¹⁰C(O)R¹¹, —C(O)NR⁸R⁹, substituted or unsubstituted C₁-C₆ alkyl, substituted or unsubstituted C₃-C₈ cycloalkyl, or substituted or unsubstituted C₃-C₈ heterocycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted alkenyl, substituted or unsubstituted alkynyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl, wherein each or which is substituted or unsubstituted with one, two, or three R¹⁴;
R⁴ is substituted or unsubstituted C₁-C₈ alkyl, substituted or unsubstituted C₂-C₈ alkenyl, substituted or unsubstituted C₁-C₈ heteroalkyl, or substituted or unsubstituted C₁-C₈ heteroalkyl, wherein each is substituted or unsubstituted with one, two, or three R¹⁴;
or R⁴ is

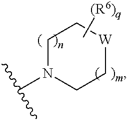

wherein
W is —CR⁶R⁶—, —O—, —S—, —NR⁵—, —S(O)₂—, or —C(O)—;
R⁵ is H or substituted or unsubstituted C₁-C₆ alkyl, substituted or unsubstituted cycloalkyl, or substituted or unsubstituted heterocycloalkyl;
each R⁶ is independently H, halogen, CN, —NR⁸R⁹, —OR¹⁰, —C(O)R¹⁰, —C(O)OR¹⁰, —NR⁸C(O)R¹¹—, —C(O)NR⁸R⁹, —SOR¹¹, —SO₂R¹¹, —SR¹¹, substituted or unsubstituted C₁-C₆ alkyl; or two R⁶ can join together with the atom(s) to which they are attached to form a C₃-C₆ cycloalkyl or C₃-C₈ heterocycloalkyl ring;
n and m are each independently 0, 1, 2, or 3; and
q is 0, 1, 2, 3, 4, 5, or 6;
R⁷ is H, halogen, —OR¹⁰, —C(O)R¹⁰, —C(O)OR¹⁰, —CN, —NR¹⁰R¹⁰, or substituted or unsubstituted C₁-C₆ alkyl, wherein the alkyl or heteroalkyl is substituted or unsubstituted with one, two, or three R¹⁴;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
R^{13A} and R^{13B} are each independently H, CF₃, halogen, or C₁-C₆ alkyl;
each R¹⁴ is independently selected from halogen, —CN, —NR⁸R⁹, —OR¹⁰, —C(O)R¹⁰, —C(O)OR¹⁰, —C(O)NR⁸R⁹, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, or C₃-C₁₀ heterocycloalkyl; and
p is 1, 2, 3, or 4.

In another aspect, provided herein is a compound having the structure of Formula (If), or a pharmaceutically acceptable salt or solvate thereof:

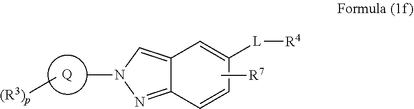

wherein,
ring Q is C₆ aryl or 5- to 10-membered heteroaryl;
L is —CR^{13A}R^{13B}, —C(O)—, —S—, —S(O)—, —S(O)₂—, or —S(O₂)NH—;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR^{c}, —C(O)NR⁸R⁹, —SOR¹¹, —SO₂R¹¹, —SO₂NR⁸R⁹, —NR¹²C(O)R¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁴ is substituted or unsubstituted C₁-C₈ alkyl, substituted or unsubstituted C₂-C₈ alkenyl, substituted or unsubstituted C₁-C₈ heteroalkyl;
or R⁴ is

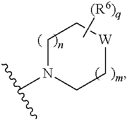

wherein
   W is —CR⁶R⁶—, —O—, —S—, —NR⁵—, —S(O)₂—, or —C(O)—;
   R⁵ is H or substituted or unsubstituted C₁-C₆ alkyl, substituted or unsubstituted cycloalkyl, or substituted or unsubstituted heterocycloalkyl;
   each R⁶ is independently H, halogen, CN, —NR⁸R⁹, —OR¹⁰, —C(O)R¹⁰, —C(O)OR¹⁰, —NR⁸C(O)R¹¹—, —C(O)NR⁸R⁹, —SOR¹¹, —SO₂R¹¹, —SR¹¹, substituted or unsubstituted C₁-C₆ alkyl;
   or two R⁶ can join together with the atom(s) to which they are attached to form a C₃-C₆ cycloalkyl or C₃-C₈ heterocycloalkyl ring;
   n and m are each independently 0, 1, 2, or 3; and
   q is 0, 1, 2, 3, 4, 5, or 6;
R⁷ is H, halogen, —OR¹⁰, —C(O)R¹⁰, —C(O)OR¹⁰, —CN, —NR¹⁰R¹⁰, or substituted or unsubstituted C₁-C₆ alkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
R^{13A} and R^{13B} are each independently H, CF₃, halogen, or C₁-C₆ alkyl; and
p is 1, 2, 3, or 4.

In some embodiments, ring Q is an aryl or heteroaryl. In some embodiments, ring Q is aryl. In some embodiments, ring Q is a bicyclic or monocyclic heteroaryl. In some embodiments, ring Q is a bicyclic heteroaryl. In some embodiments, ring Q is a monocyclic heteroaryl. In some embodiments, ring Q is a 5- to 6-membered heteroaryl.

In some embodiments, ring Q is C₆ aryl. In some embodiments, ring Q is a 6-membered monocyclic heteroaryl. In some embodiments, ring Q is phenyl or a 6-membered monocyclic heteroaryl. In some embodiments, ring Q is pyridinyl, pyrazinyl, pyrimidinyl, or pyridazinyl. In some embodiments, ring Q is phenyl or pyridinyl. In some embodiments, ring Q is phenyl. In some embodiments, ring Q is pyridinyl. In some embodiments, ring Q is pyrazinyl. In some embodiments, ring Q is pyrimidinyl. In some embodiments, ring Q is pyridazinyl.

In some embodiments, ring Q is:

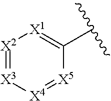

wherein,
X¹, X², X³, X⁴, and X⁵ are each independently N or CR³; and wherein at least two of X¹—X⁵ is CR³.

In some embodiments, X² is N; and X¹, X³, X⁴, and X⁵ are each CR³. In some embodiments, X³ is N; and X¹, X², X⁴, and X⁵ are each CR³. In some embodiments, X¹ is N; and X², X³, X⁴, and X⁵ are each CR³. In some embodiments, X² and X⁴ are each N; and X¹, X³ and X⁵ are each CR³. In some embodiments, X² and X³ are each N; and X¹, X⁴ and X⁵ are each CR³. In some embodiments, X¹ and X⁴ are each N; and X², X³ and X⁵ are each CR³. In some embodiments, X¹, X², and X⁴ are N; and X³ and X⁵ are each CR³.

In some embodiments, ring Q is:

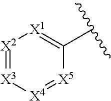

wherein,
X¹ and X⁵ are each independently N or CH;
X² is N or CR^{3A};
X³ is N or CR^{3B};
X⁴ is N, NR^{3C}, or CR^{3C}; and
R^{3A}, R^{3B}, and R^{3C} are each independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl.

In some embodiments, the compound of Formula (I) has the structure of Formula (III), or a pharmaceutically acceptable salt or solvate thereof:

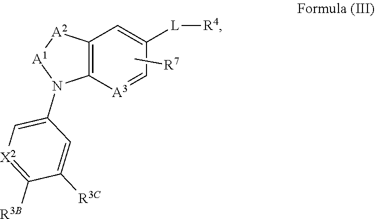

wherein,
X² is N or CR^{3A}; and
R^{3A}, R^{3B}, and R^{3C} are each independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
provided that R^{3A}, R^{3B}, and R^{3C} are not all H at the same time.

In some embodiments, the compound of Formula (Ie) has the structure of Formula (IIIe), or a pharmaceutically acceptable salt or solvate thereof:

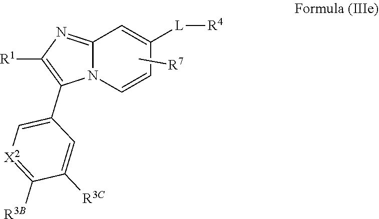

wherein,
X² is N or CR^{3A}; and
R^{3A}, R^{3B}, and R^{3C} are each independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
provided that R^{3A}, R^{3B}, and R^{3C} are not all H at the same time.

In some embodiments, X² is N. In some embodiments, X² is CR^{3A}. In some embodiments, X² is CH.

In some embodiments, R^{3B} is H or halogen; and R^{3C} is selected from substituted or unsubstituted C₃-C₈ heterocycloalkyl, or substituted or unsubstituted 5-membered heteroaryl. In some embodiments, R^{3B} is H or halogen; and R^{3C} is substituted or unsubstituted 5-membered heteroaryl. In some embodiments, R^{3B} is H, Br, Cl, or F; and R^{3C} is substituted or unsubstituted 5-membered heteroaryl.

In some embodiments, R^{3C} is H or halogen; and R^{3B} is selected from substituted or unsubstituted C₃-C₈ heterocycloalkyl, or substituted or unsubstituted 5-membered heteroaryl. In some embodiments, R^{3C} is H or halogen; and R^{3B} is substituted or unsubstituted 5-membered heteroaryl. In some embodiments, R^{3C} is H, Br, Cl, or F; and R^{3B} is substituted or unsubstituted 5-membered heteroaryl.

In some embodiments, R^{3B} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl. In some embodiments, R^{3B} is selected from H, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₃-C₈ cycloalkyl, substituted or unsubstituted C₃-C₈ heterocycloalkyl, and substituted or unsubstituted 5-membered heteroaryl. In some embodiments, R^{3B} is selected from —C(O)NR⁸R⁹, —NR¹²C(O)NR⁸R⁹, —NR¹²C(O)OR¹⁰, —OC(O)NR⁸R⁹. In some embodiments, R^{3B} is selected from substituted or unsubstituted C₃-C₈ heterocycloalkyl. In some embodiments, R^{3B} is selected from substituted or unsubstituted 5-membered heteroaryl. In some embodiments, the heteroaryl is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, R^{3B} is H.

In some embodiments, R^{3C} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl. In some embodiments, R^{3C} is selected from H, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₃-C₈ cycloalkyl, substituted or unsubstituted C₃-C₈ heterocycloalkyl, and substituted or unsubstituted 5-membered heteroaryl. In some embodiments, R^{3C} is selected from —C(O)NR⁸R⁹, —NR¹²C(O)NR⁸R⁹, —NR¹²C(O)OR¹⁰, —OC(O)NR⁸R⁹. In some embodiments, R^{3C} is selected from substituted or unsubstituted C₃-C₈ heterocycloalkyl. In some embodiments, R^{3C} is selected from substituted or unsubstituted 5-membered heteroaryl. In some embodiments, the heteroaryl is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, R^{3C} is H.

In some embodiments, ring Q is a 5-membered heteroaryl. In some embodiments, ring Q is triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl.

In some embodiments, ring Q is:

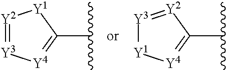

wherein,
Y¹ is O, S, or NR^{3D};
Y² is N or CR^{3A};
Y³ and Y⁴ are each independently N or CR^{3B};
R^{3A} and R^{3B} are each independently selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl; and
R^{3D} is H or C₁-C₆ alkyl.

In some embodiments, Y¹ is O or S; Y² is CR^{3A}; and Y³ and Y⁴ are each independently N or CR^{3B}. In some embodiments, Y¹ is 0; Y² is CR^{3A}; and Y³ and Y⁴ are each independently N or CR^{3B}. In some embodiments, Y¹ is S; Y² is CR^{3A}; and Y³ and Y⁴ are each independently N or CR^{3B}.

In some embodiments, Y¹ is O or S; Y² is N; and Y³ and Y⁴ are each independently N or CR^{3B}. In some embodiments, Y¹ is 0; Y² is N; and Y³ and Y⁴ are each independently N or CR^{3B}. In some embodiments, Y¹ is S; Y² is N; and Y³ and Y⁴ are each independently N or CR^{3B}.

In some embodiments, R^{3A}, R^{3B}, and R^{3C} are each independently selected from H, halogen, —NR⁸R⁹, —OR¹⁰, —C(O)R¹⁰, —C(O)OR¹⁰, —C(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl. In some embodiments, R^{3A}, R^{3B}, and R^{3C} are each independently selected from —NR⁸R⁹, —OR¹⁰, —C(O)R¹⁰, —C(O)OR¹⁰, —C(O)NR⁸R⁹. In some embodiments, R^{3A}, R^{3B}, and R^{3C} are each independently selected from substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl. In some embodiments, R^{3A}, R^{3B}, and R^{3C} are each independently selected from substituted or unsubstituted 5-membered heteroaryl. In some embodiments, R^{3A}, R^{3B}, and R^{3C} are each independently selected from H or halogen.

In some embodiments, R^{3D} is H. In some embodiments, R^{3D} is C₁-C₆ alkyl.

In some embodiments, the compound has the structure of Formula (IVa) or (IVb), or a pharmaceutically acceptable salt or solvate thereof:

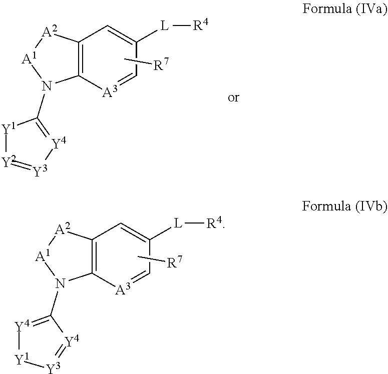

In some embodiments, the compound has the structure of Formula (IVc) or (IVd), or a pharmaceutically acceptable salt or solvate thereof:

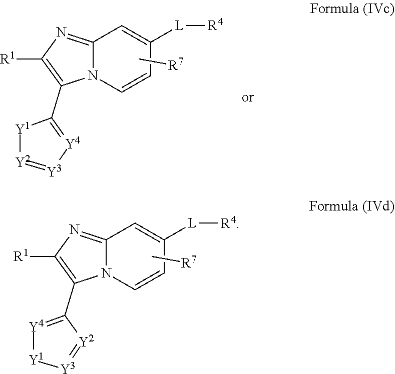

In some embodiments, each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)NR⁸R⁹, —NR¹²C(O)OR¹⁰, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl, substituted or unsubstituted C₂-C₈ alkenyl, or C₂-C₈ substituted or unsubstituted alkynyl. In some embodiment, each R³ is independently selected from H, halogen, —NR⁸R⁹, —OR¹⁰, CN, —C(O)R¹⁰, —C(O)OR¹⁰, —C(O)NR⁸R⁹, —NR¹²C(O)OR¹⁰, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5-membered heteroaryl.

In some embodiments, each R³ is independently selected from H, Cl, F, substituted or unsubstituted C₃-C₈ heterocycloalkyl, or substituted or unsubstituted 5-membered heteroaryl.

In some embodiments, each R³ is independently selected from substituted or unsubstituted C₃-C₈ heterocycloalkyl or substituted or unsubstituted 5-membered heteroaryl, substituted or unsubstituted with one or two —NH₂, CF₃, C₁-C₆ alkyl, or C₃-C₈ cycloalkyl. In some embodiments, each R³ is independently a substituted or unsubstituted 5-membered heteroaryl. In some embodiments, each R³ is independently selected from triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, each R³ is independently triazinyl, pyrrolyl, imidazolyl, pyrazolyl, oxazolyl, isoxazolyl, thiophenyl, thiazolyl, isothiazolyl, triazolyl, oxadiazolyl, thiadiazolyl, or tetrazolyl, each of which is substituted or unsubstituted with one or two halogen, —NH₂, CF₃, C₁-C₆ alkyl, or C₃-C₈ cycloalkyl.

In some embodiments, each R³ is independently selected from the group consisting of

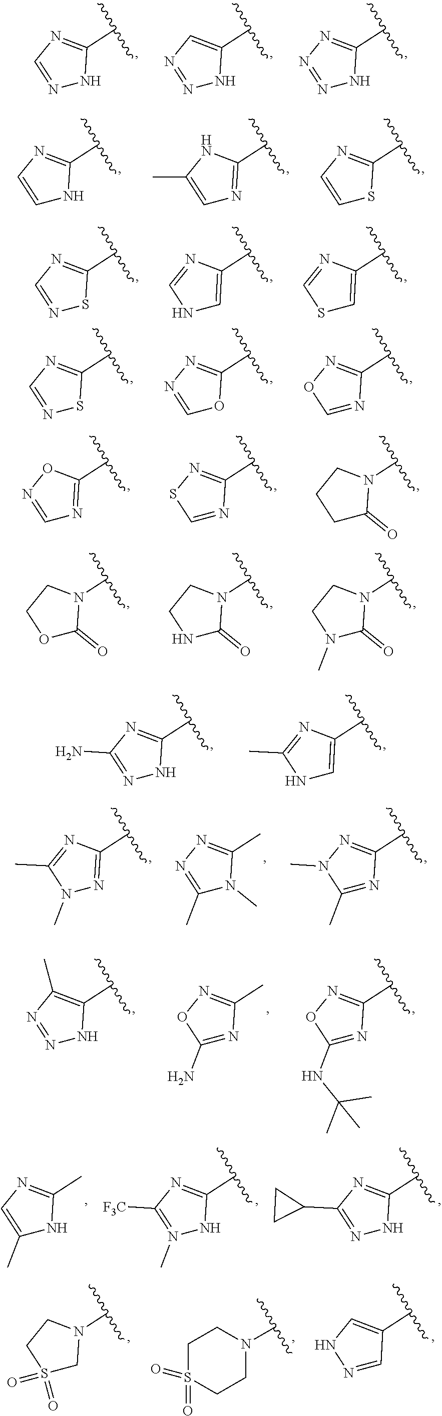

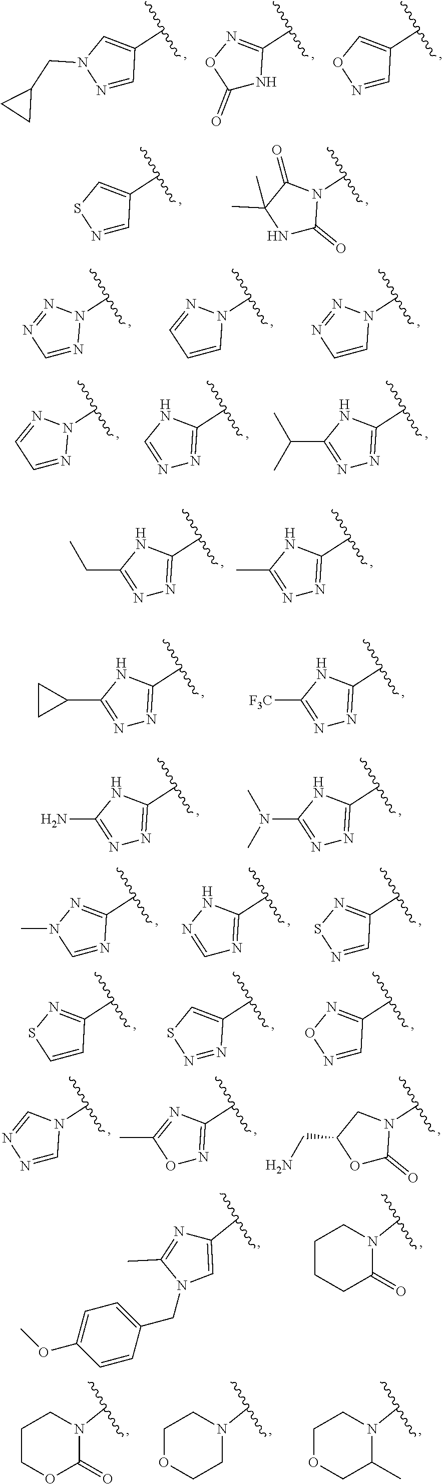

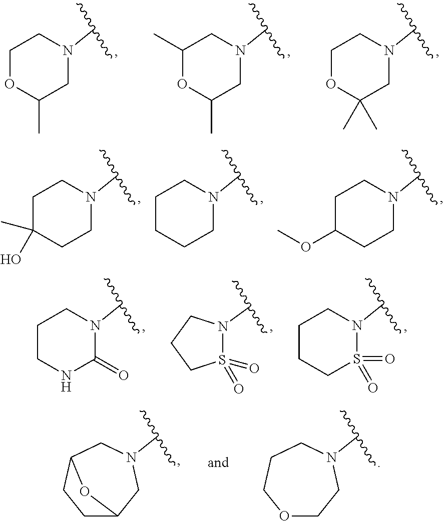

In some embodiments, each R³ is independently selected from the group consisting of

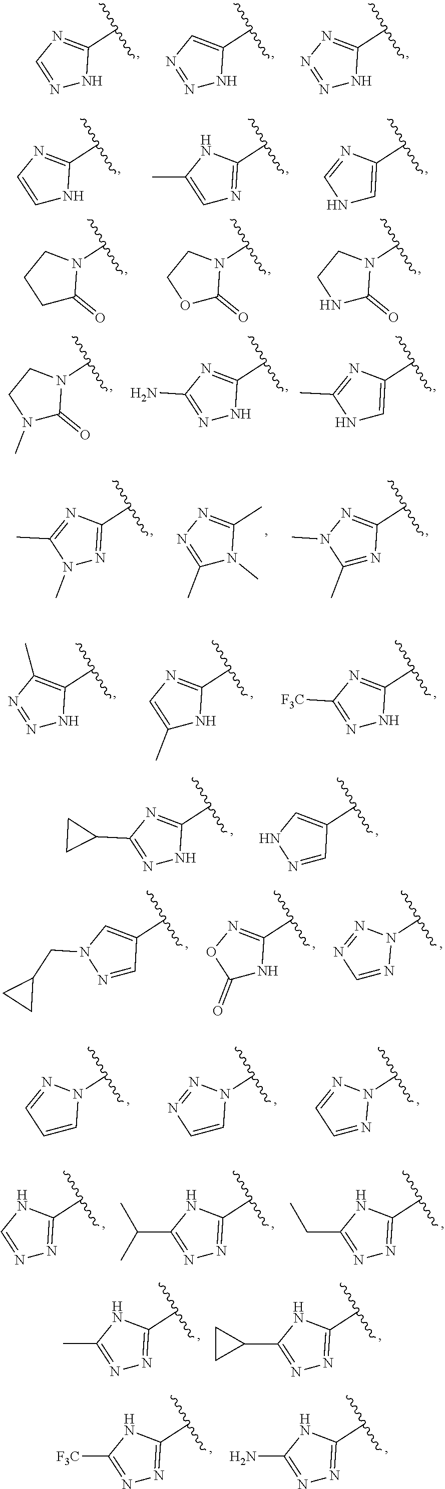

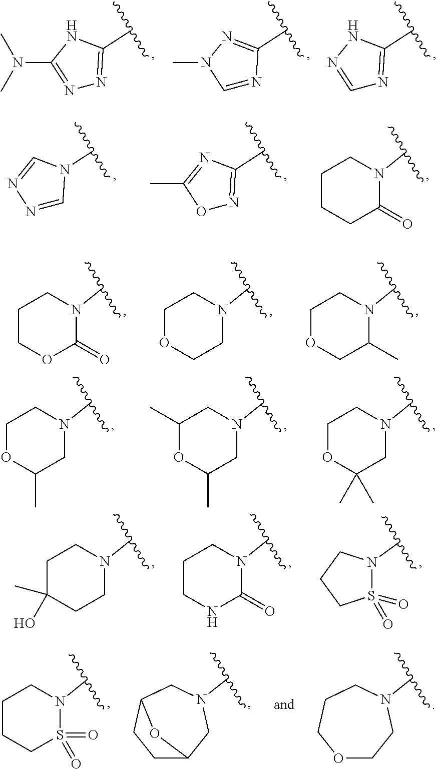

In some embodiments, each R³ is independently —C(O)R¹⁰, —C(O)OR¹⁰, —C(O)NR⁸R⁹, or —NR¹²C(O)OR¹⁰. In some embodiments, each R³ is independently —C(O)R¹⁰, —C(O)NR⁸R⁹, or —NR¹²C(O)OR¹⁰. In some embodiments, each R³ is independently —C(O)R¹⁰. In some embodiments, each R³ is independently —C(O)OR¹⁰. In some embodiments, each R³ is independently-C(O)NR⁸R⁹. In some embodiments, each R³ is independently —NR¹²C(O)OR¹⁰.

In some embodiments, each R³ is independently selected from

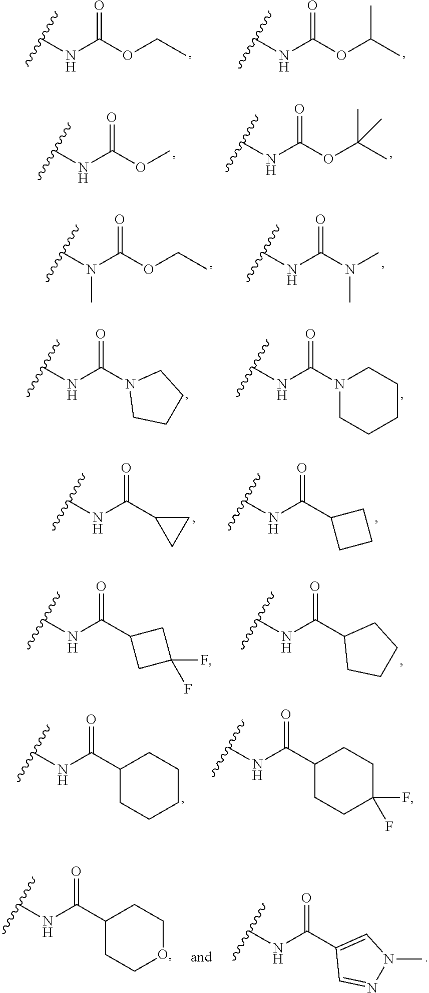

In some embodiments, the definitions of R³ are each independently the same as the definitions of R^{3A}, R^{3B}, and R^{3C}. In some embodiments, the definitions of R³ are the same as the definitions of R^{3A}. In some embodiments, the definitions of R³ are the same as the definitions of R^{3B}. In some embodiments, the definitions of R³ are the same as the definitions of R^{3C}.

In some embodiments, ring Q is a bicyclic heteroaryl. In some embodiments, ring Q is a bicyclic heteroaryl comprising 1-3 heteroatoms selected from N, O, and S atoms. In some embodiments, ring Q is a bicyclic heteroaryl comprising 1, 2, or 3 N atoms. In some embodiments, ring Q is [1,2,4]triazolo[1,5-a]pyridine.

In some embodiments, ring Q is

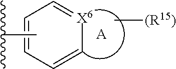

wherein,
ring A is a 5-membered heteroaryl optionally comprising 1 or 2 N atoms;
X⁶ is C or N; and
R¹⁵ is H, halogen, —NR⁸R⁹, -substituted or unsubstituted C₁-C₆ alkyl, substituted or unsubstituted C₁-C₆ haloalkyl, substituted or unsubstituted C₃-C₈ cycloalkyl, or substituted or unsubstituted C₃-C₈ heterocycloalkyl.

In some embodiments, ring A is imidazolyl, pyrazolyl, oxazolyl, isoxazolyl, thiophenyl, thiazolyl, isothiazolyl, triazolyl, oxadiazolyl, thiadiazolyl, or tetrazolyl. In some embodiments, ring Q is pyrazolyl. In some embodiments, ring Q is imidazolyl. In some embodiments, ring Q is triazolyl.

In some embodiments, X⁶ is C. In some embodiments, X⁶ is N.

In some embodiments, R¹⁵ is H, halogen, —NR⁸R⁹, —C₁-C₆ alkyl, or C₁-C₆ haloalkyl. In some embodiments, R¹⁵ is H. In some embodiments, R¹⁵ is —NR⁸R⁹. In some embodiments, R¹⁵ is —NH₂, —NHCH₃, or —N(CH₃)₂. In some embodiments, R¹⁵ is —C₁-C₆ alkyl or C₁-C₆ haloalkyl. In some embodiments, R¹⁵ is —CH₃, —CH₂CH₃, —CH(CH₃)₂, —C(CH₃)₃, —CF₃, or CHF₂.

In some embodiments, R¹⁵ is substituted or unsubstituted C₃-C₈ cycloalkyl. In some embodiments, R¹⁵ is substituted or unsubstituted C₃-C₈ heterocycloalkyl. In some embodiments, R¹⁵ is

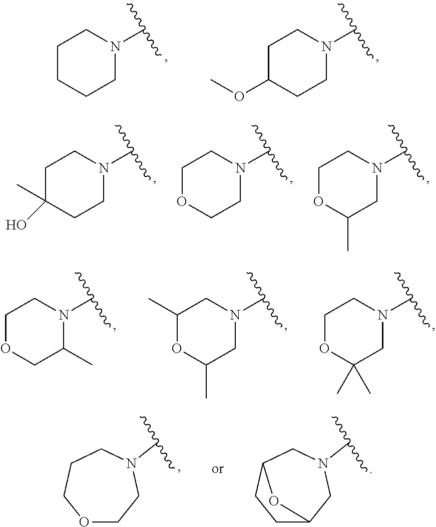

In some embodiments, L is —S—, —S(O)—, or —S(O)₂—. In some embodiments, L is —S—. In some embodiments, L is —S(O)—. In some embodiments, L is —S(O)₂—. In some embodiments, L is —C(O)—. In some embodiments, L is —O—. In some embodiments, L is —CR⁶R⁶—. In some embodiments, L is —C(O)R¹⁰—.

In some embodiments, L is —S—, —S(O)—, or —S(O)₂—; and R⁴ is substituted or unsubstituted C₁-C₈ alkyl, substituted or unsubstituted C₂-C₈ alkenyl, substituted or unsubstituted C₁-C₈ heteroalkyl.

In some embodiments, L is —S—, —S(O)—, or —S(O)₂—; and R⁴ is substituted or unsubstituted C₁-C₆ alkyl.

In some embodiments, R⁴ is substituted or unsubstituted C₁-C₈ alkyl, substituted or unsubstituted C₂-C₈ alkenyl, substituted or unsubstituted C₁-C₈ heteroalkyl. In some embodiments, R⁴ is substituted or unsubstituted C₁-C₈ alkyl. In some embodiments, R⁴ is substituted or unsubstituted C₂-C₈ alkenyl. In some embodiments, R⁴ is substituted or unsubstituted C₁-C₈ heteroalkyl.

In some embodiments, L is C(O); and R⁴ is

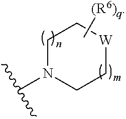

In another aspect, provided herein is a compound having the structure of Formula (V), or a pharmaceutically acceptable salt or solvate thereof:

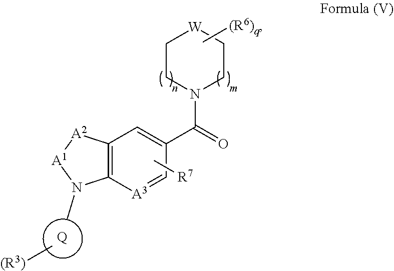

wherein,
ring Q is C₆ aryl or 5 or 10-membered heteroaryl;
A¹ is CR¹ or N and A² is CR² or N, provided that at least one of A¹ or A² is N;
A³ is N or CR⁷;
W is —CR⁶R⁶—, —O—, —S—, —NR⁵—, —S(O)₂—, or —C(O)—;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁵ is H or C₁-C₆ alkyl; and
each R⁶ is independently CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
or two R⁶ can join together with the atom(s) to which they are attached to form a C₃-C₆ cycloalkyl or C₃-C₈ heterocycloalkyl ring;
R⁷ is H, halogen, —OR¹⁰, —C(O)R¹⁰, —C(O)OR¹⁰, —CN, —C(O)NR⁸R⁹, —NR⁸C(O)R⁹, substituted or unsubstituted C₁-C₆ alkyl, or substituted or unsubstituted C₃-C₈ cycloalkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
n and m are each independently 0, 1, 2, or 3;
q is 0, 1, or 2; and
p is 1, 2, or 3.

In some embodiments, the compound has the structure of Formula (VI), or a pharmaceutically acceptable salt or solvate thereof:

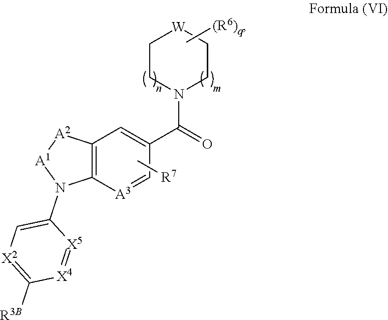

wherein,
X² is N, NR^{3A}, or CR^{3A};
X³ is N or CR^{3B};
X⁴ is N, NR^{3C}, or CR^{3C}; and
R^{3A}, R^{3B}, and R^{3C} are each independently H, halogen, —CN, —NR⁸R⁹, —OR¹⁰, CN, —C(O)R¹⁰, —C(O)OR¹, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R^{3A} and R^{3B} together with the atoms to which they are attached form a substituted or unsubstituted 5 to 6-membered aryl or heteroaryl; or
R^{3B} and R^{3C} together with the atoms to which they are attached form a substituted or unsubstituted 5 to 6-membered aryl or heteroaryl; and
wherein CR^{3A}, CR^{3B}, and CR^{3C} are not all H at the same time.

In some embodiments, the compound has the structure of Formula (VIa), or a pharmaceutically acceptable salt or solvate thereof:

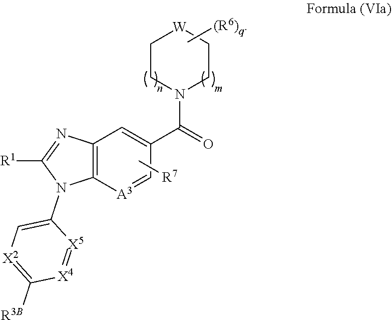

In some embodiments, the compound has the structure of Formula (VIb), or a pharmaceutically acceptable salt or solvate thereof:

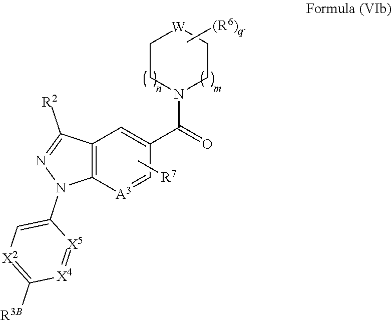

In some embodiments, the compound has the structure of Formula (VIc), or a pharmaceutically acceptable salt or solvate thereof:

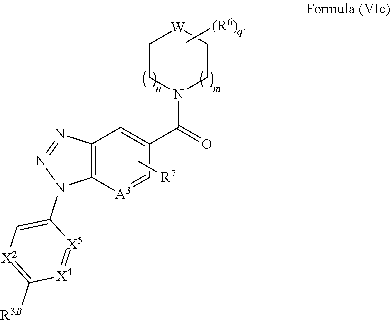

In some embodiments, X⁴ is N and X⁵ is CH. In some embodiments, X⁴ is CR^{3C} and X⁵ is N. In some embodiments, X⁴ is CR^{3C} and X⁵ is CH.

In some embodiments, X² is N and X⁴ is CR^{3C}. In some embodiments, R^{3B} is H, and R^{3C} is —C(O)R¹⁰, —C(O)OR¹⁰, —C(O)NR⁸R⁹, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3C} is H, and R^{3B} is —C(O)R¹⁰, —C(O)OR^{c}, —C(O)NR⁸R⁹, —NR¹²C(O)R¹⁰, —NR¹²C(O)OR¹⁰, substituted or unsubstituted C₃-C₈ heterocycloalkyl, or substituted or unsubstituted 5-membered heteroaryl.

In some embodiments, X² is C^{3A} and X⁴ is N. In some embodiments, R^{3A} is H, and R^{3B} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3B} is H, and R^{3A} is —C(O)R¹⁰, —C(O)OR¹⁰, —C(O)NR⁸R⁹, —NR¹²C(O)R¹⁰, —NR¹²C(O)OR¹⁰, substituted or unsubstituted C₃-C₈ heterocycloalkyl, or substituted or unsubstituted 5-membered heteroaryl.

In some embodiments, X² is CR^{3A} and X⁴ is CR^{3C}. In some embodiments, R^{3A} and R^{3B} are each H; and R^{3C} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3A} and R^{3C} are each H; and R^{3B} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl. In some embodiments, R^{3B} and R^{3C} are each H; and R^{3A} is —C(O)R¹⁰, —NR¹²C(O)R¹⁰, —NR¹²C(O)OR¹⁰, substituted or unsubstituted C₃-C₈ heterocycloalkyl, or substituted or unsubstituted 5-membered heteroaryl.

In some embodiments, the compound has the structure of Formula (VII), or a pharmaceutically acceptable salt or solvate thereof:

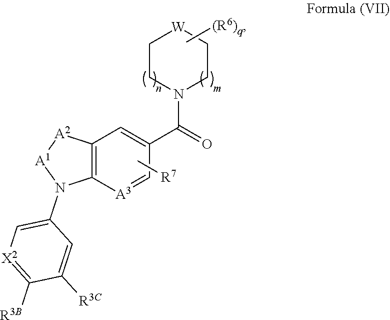

wherein,
X² is N or CH; and
R^{3C} is H or halogen and R^{3B} is selected from H, halogen, —CN, —NR⁸R⁹, —OR¹⁰, —C(O)R¹⁰, —C(O)OR¹, —C(O)NR⁸R⁹, —NR¹²C(O)NR⁸R⁹, —NR¹²C(O)OR¹⁰, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl; or
R^{3B} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

In some embodiments, the compound has the structure of Formula (VIIa), or a pharmaceutically acceptable salt or solvate thereof:

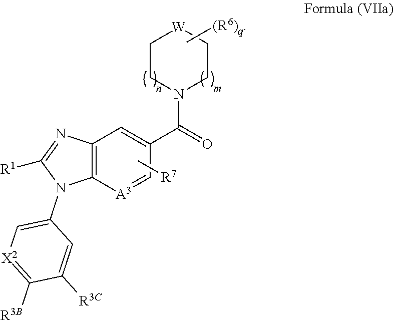

In some embodiments, the compound has the structure of Formula (VIIb), or a pharmaceutically acceptable salt or solvate thereof:

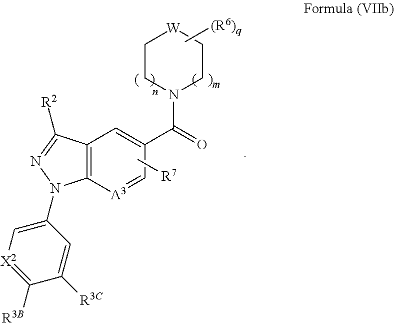

In some embodiments, the compound has the structure of Formula (VIIc), or a pharmaceutically acceptable salt or solvate thereof:

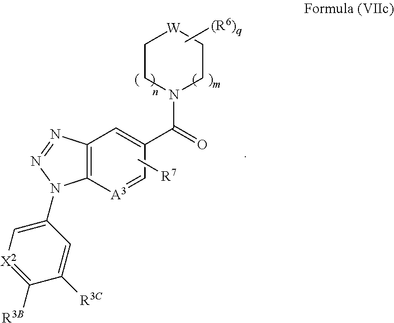

In some embodiments, the compound has the structure of Formula (VIIIa), (VIIIb), (VIIIc), (VIIId), (VIIIe), or (VIIIf), or a pharmaceutically acceptable salt or solvate thereof:

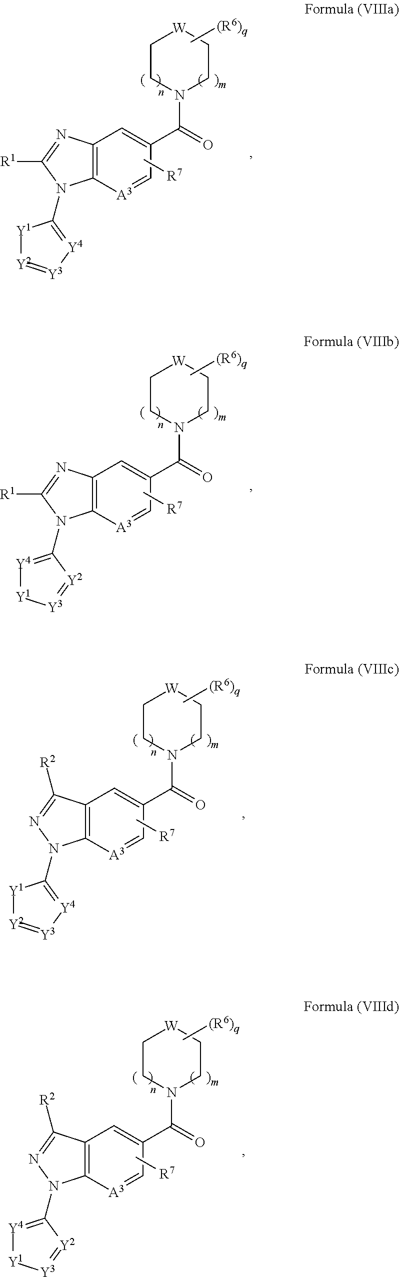

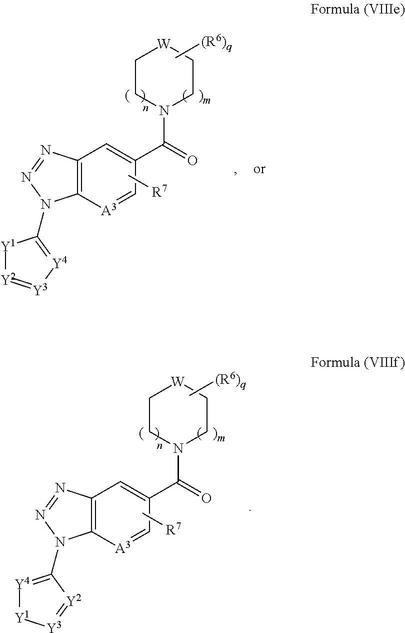

In another aspect, provided herein is a compound of Formula (IX), or a pharmaceutically acceptable salt or solvate thereof:

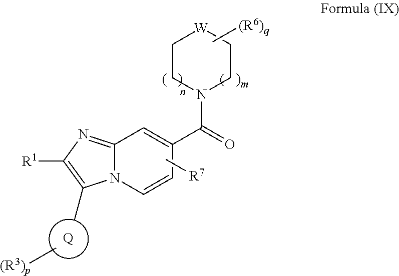

wherein,
ring Q is C₆ aryl or 5 or 10-membered heteroaryl;
W is —CR⁶R⁶—, —O—, —S—, —NR⁵—, —S(O)₂—, or —C(O)—;
R¹ is H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁵ is H or C₁-C₆ alkyl; and
each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
or two R⁶ can join together with the atom(s) to which they are attached to form a C₃-C₆ cycloalkyl or C₃-C₈ heterocycloalkyl ring;
R⁷ is H, halogen, —OR¹⁰, —C(O)R¹⁰, —C(O)OR¹⁰, —CN, —C(O)NR⁸R⁹, —NR⁸C(O)R⁹, substituted or unsubstituted C₁-C₆ alkyl, or substituted or unsubstituted C₃-C₈ cycloalkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
n and m are each independently 0, 1, 2, or 3;
q is 0, 1, or 2; and
p is 1, 2, or 3.

In some embodiments, the compound has the structure of Formula (X), or a pharmaceutically acceptable salt or solvate thereof:

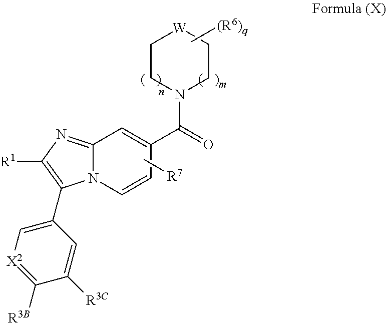

X² is N or CR^{3A}; and
R^{3A}, R^{3B}, and R^{3C} are each independently selected from H, halogen, —CN, —NR⁸R⁹, —C(O)R¹⁰, —C(O)OR¹⁰, —C(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, or substituted or unsubstituted 5-membered heteroaryl;
provided that R^{3A}, R^{3B}, and R^{3C} are not all H at the same time.

In some embodiments, the compound has the structure of Formula (XIa) or (XIb), or a pharmaceutically acceptable salt or solvate thereof:

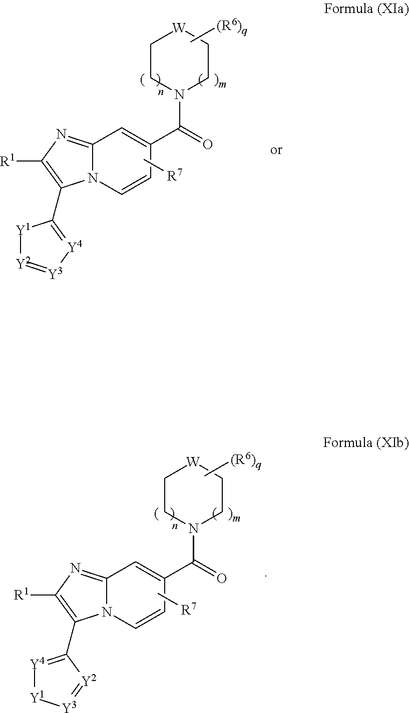

In some embodiments, the compound has the structure of Formula (XII), or a pharmaceutically acceptable salt or solvate thereof:

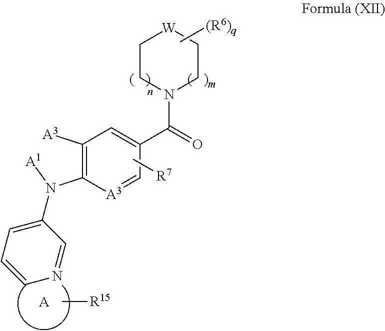

wherein,
ring A is a 5-membered heteroaryl optionally comprising 1 or 2 N atoms; and
R¹⁵ is H, halogen, —NR⁸R⁹, -substituted or unsubstituted C₁-C₆ alkyl, substituted or unsubstituted C₁-C₆ haloalkyl, substituted or unsubstituted C₃-C₈ cycloalkyl, or substituted or unsubstituted C₃-C₈ heterocycloalkyl.

In some embodiments, W is —CR⁶R⁶—, —O—, —S—, —NR⁵—, —S(O)—, or —S(O)₂—. In some embodiments, W is —O—, —S— or —S(O)₂—. In some embodiments, W is —O—. In some embodiments, W is —S—. In some embodiments, W is —NR⁵—. In some embodiments, W is —S(O)₂—. In some embodiments, W is —S(O)—.

In some embodiments, W is —CR⁶R⁶—. In some embodiments, W is —CH₂—. In some embodiments, W is —CF₂—. In some embodiments, W is —CHF—.

In some embodiments, each R⁶ is independently H, halogen, CN, —NR⁸R⁹, —OR¹⁰, —C(O)R¹⁰, —C(O)OR¹⁰, —C(O)NR⁸R⁹, —NR⁸C(O)R¹¹—, —SOR¹¹, —SO₂R¹¹, —SR¹¹, substituted or unsubstituted C₁-C₆ alkyl, substituted or unsubstituted C₁-C₆ haloalkyl, or C₃-C₈ cycloalkyl. In some embodiments, each R is independently H, halogen, CN, —NR⁸R⁹, —OR¹⁰, —C(O)R¹⁰, —C(O)OR¹⁰, —C(O)NR⁸R⁹, —NR⁸C(O)R¹¹—, —SOR¹¹, —SO₂R¹¹, —SR¹¹, substituted or unsubstituted C₁-C₆ alkyl, or C₃-C₈ cycloalkyl.

In some embodiments, each R⁶ is independently H, halogen, CN, —NR⁸R⁹, —OR¹⁰, —C(O)R¹⁰, —C(O)OR¹⁰, —C(O)NR⁸R⁹, or substituted or unsubstituted C₁-C₆ alkyl. In some embodiments, each R⁶ is halogen.

In some embodiments, each R⁶ is independently F, —NH₂, —CF₃, —CHF₂, —CF₂CH₃, —OH, —OCH₃, or —CH₃. In some embodiments, each R⁶ is independently F, —NH₂, —OH, —OCH₃, or —CH₃. In some embodiments, each R⁶ is independently F. In some embodiments, each R⁶ is independently —CH₃.

In some embodiments, two R⁶ can join together with the atom(s) to which they are attached to form a C₃-C₆ cycloalkyl ring. In some embodiments, two R⁶ on the same carbon atom can join together to form a cycloalkyl ring. In some embodiments, two R⁶ on different carbon atoms can join together to form a cycloalkyl ring. In some embodiments, the ring is a spirocycle. In some embodiments, two R⁶ join together to form a cyclopropyl, cyclobutyl, cyclopentyl or cyclohexyl. In some embodiments, two R⁶ join together to form a cyclopropyl.

In some embodiments, R⁷ is H, halogen, —OR¹⁰, —C(O)R¹⁰, —C(O)OR¹⁰, or substituted or unsubstituted C₁-C₆ alkyl. In some embodiments, R⁷ is H.

In some embodiments,

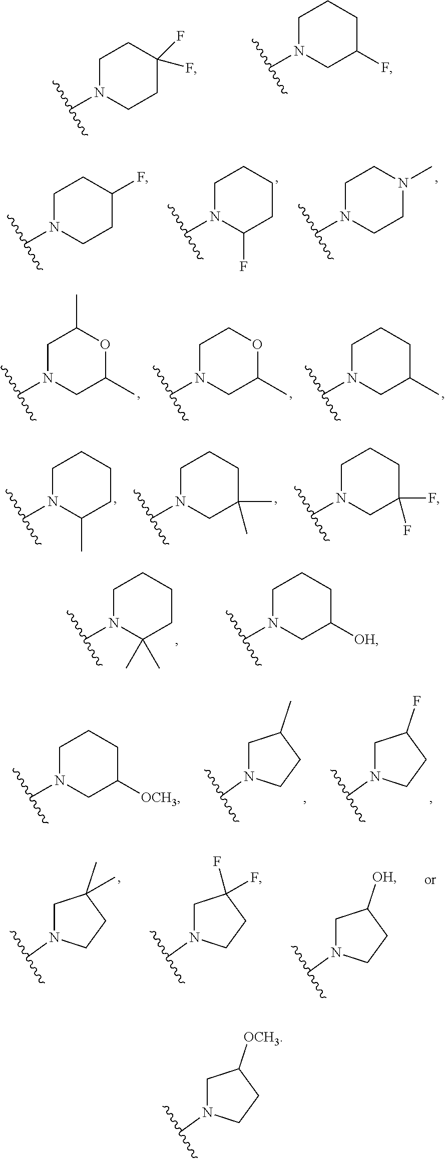

is

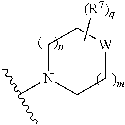

In some embodiments,

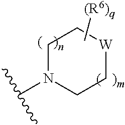

is

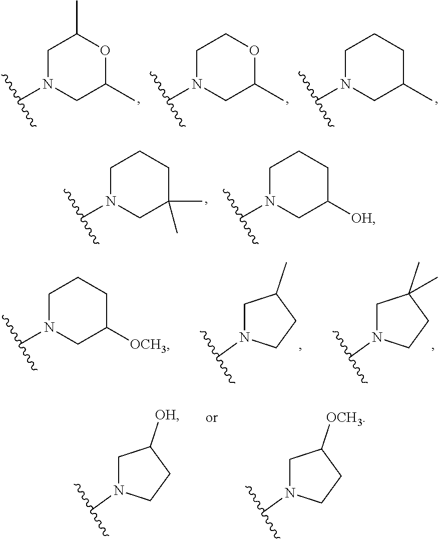

In some embodiments

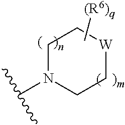

is

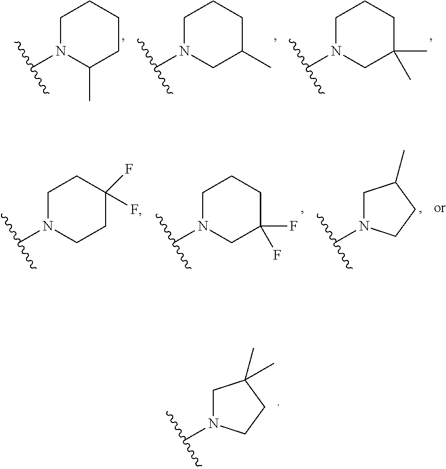

In some embodiments,

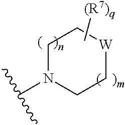

is

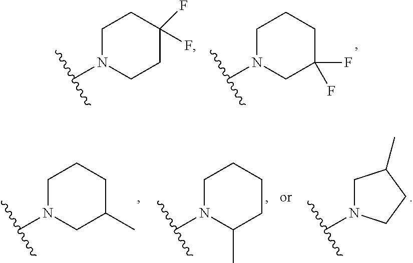

In some embodiments,

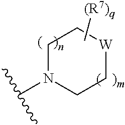

is

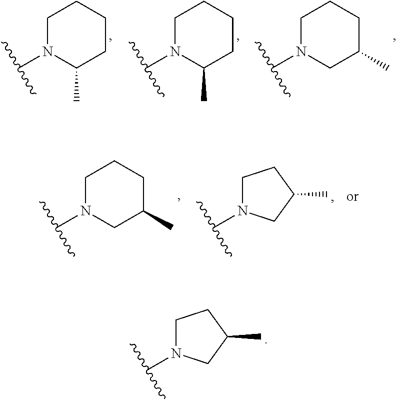

In some embodiments,

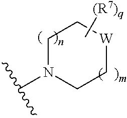

is

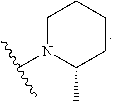 [SMILES: *N1CCCC[C@@H]1C]

In some embodiments,

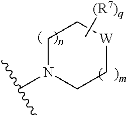

is

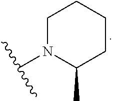 [SMILES: *N1CCCC[C@H]1C]

In some embodiments,

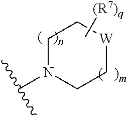

is

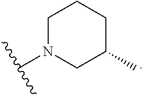 [SMILES: *N1CCC[C@H](C)C1]

In some embodiments,

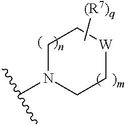

is

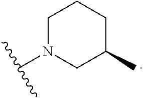 [SMILES: *N1CCC[C@@H](C)C1]

In some embodiments,

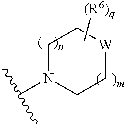

is

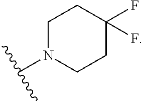 [SMILES: *N1CCC(F)(F)CC1]

In some embodiments,

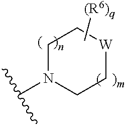

is

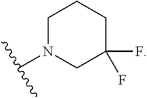 [SMILES: *N1CCCC(F)(F)C1]

In some embodiments,

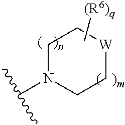

is

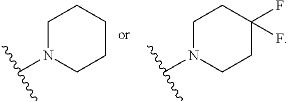 [SMILES: *N1CCCCC1 ; *N1CCC(F)(F)CC1]

In some embodiments, each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl. In some embodiments, each R⁸ and R⁹ are independently selected at each occurrence from H or C₁-C₆ alkyl. In some embodiments, each R⁸ and R⁹ are independently selected at each occurrence from H. In some embodiments, each R⁸ and R⁹ are independently selected at each occurrence from C₁-C₆ alkyl.

In some embodiments, each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl. In some embodiments, each R¹⁰ is independently selected from H or C₁-C₆ alkyl. In some embodiments, each R¹⁰ is independently selected from C₁-C₆ haloalkyl. In some embodiments, each R¹⁰ is independently selected from C₃-C₈ cycloalkyl. In some embodiments, each R¹⁰ is independently selected from C₆-C₁₀ aryl and 5- to 10-membered heteroaryl. In some embodiments, each R¹⁰ is independently 5-membered heteroaryl.

In some embodiments, each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl. In some embodiments, each R¹¹ is independently selected from C₁-C₆ alkyl. In some embodiments, each R¹¹ is selected from C₁-C₆ haloalkyl. In some embodiments, each R¹¹ is selected from C₃-C₈ cycloalkyl. In some embodiments, each R¹¹ is selected from C₆-C₁₀ aryl, and 5- to 10-membered heteroaryl.

In some embodiments, each R¹² is independently selected from H, C₁-C₆ alkyl, C₂-C₆ alkenyl, C₁-C₆ haloalkyl, and C₃-C₈ cycloalkyl. In some embodiments, each R¹² is independently selected from H or C₁-C₆ alkyl. In some embodiments, each R¹² is independently selected from C₁-C₆ haloalkyl. In some embodiments, each R¹² is independently selected from C₃-C₈ cycloalkyl.

In some embodiments, each R¹⁴ is independently selected from —CN, —NR⁸R⁹, —OR¹⁰, —C(O)R¹⁰, —C(O)OR¹⁰, —C(O)NR⁸R⁹, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl, or C₃-C₁₀ heterocycloalkyl. In some embodiments, each R¹⁴ is independently selected from —NR⁸R⁹ or —OR¹⁰. In some embodiments, each R¹⁴ is independently selected from —C(O)R′⁰, —C(O)OR¹⁰, or —C(O)NR⁸R⁹. In some embodiments, each R¹⁴ is independently selected from C₁-C₆ alkyl, C₂-C₆ alkenyl, C₂-C₆ alkynyl, C₁-C₆ heteroalkyl, or C₁-C₆ haloalkyl. In some embodiments, each R¹⁴ is independently selected from C₃-C₁₀ cycloalkyl or C₃-C₁₀ heterocycloalkyl.

In some embodiments, p is 1, 2, 3, or 4. In some embodiments, p is 2 or 3. In some embodiments, p is 3. In some embodiments, p is 5. In some embodiments, p is 4. In some embodiments, p is 3. In some embodiments, p is 2. In some embodiments, p is 1.

In some embodiments, q is q is 0, 1, 2, 3, 4, 5, or 6. In some embodiments, q is 1 or 2. In some embodiments, q is 6. In some embodiments, q is 5. In some embodiments, q is 4. In some embodiments, q is 3. In some embodiments, q is 2. In some embodiments, q is 1. In some embodiments, q is 0.

In some embodiments, n and m are independently 0, 1, or 2. In some embodiments, n and m are independently 0. In some embodiments, n and m are independently 1. In some embodiments, n and m are independently 2. In some embodiments, n is 0, 1, or 2; and m is 1 or 2. In some embodiments, n is 0; and
m is 1 or 2. In some embodiments, n is 1; and m is 1 or 2. In some embodiments, n is 2; and m is 1 or 2.

In some embodiments, the PDGH inhibitor is a compound presented in Table 1a, or pharmaceutically acceptable salt or solvate thereof.

Table 1a. Representative sulfoxide PGDH inhibitors.

TABLE 1a
Representative sulfoxide PGDH inhibitors.

| Compound No | Structure |
|---|---|
| C-1 | 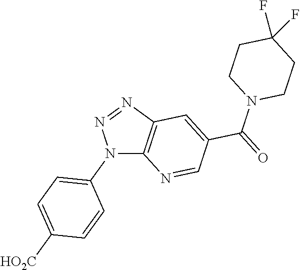 [SMILES: O=C(O)c1ccc(-n2nnc3cc(C(=O)N4CCC(F)(F)CC4)cnc32)cc1] |
| C-2 | 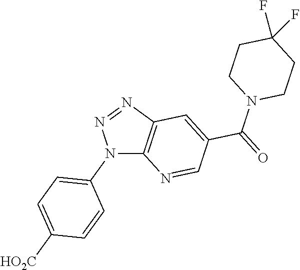 [SMILES: O=C(O)c1ccc(-n2nnc3cc(C(=O)N4CCC(F)(F)CC4)cnc32)cc1] |
| C-3 | 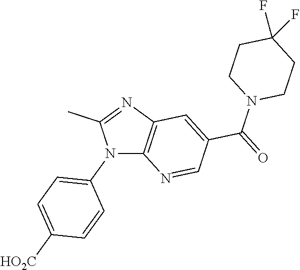 [SMILES: Cc1nc2cc(C(=O)N3CCC(F)(F)CC3)cnc2n1-c1ccc(C(=O)O)cc1] |
| C-4 | 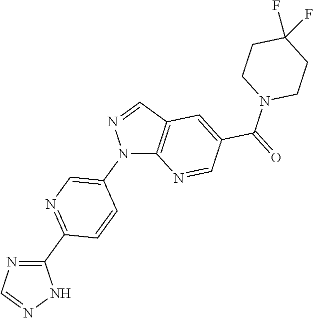 [SMILES: O=C(c1cnc2c(cnn2-c2ccc(-c3ncn[nH]3)nc2)c1)N1CCC(F)(F)CC1] |
| C-5 | 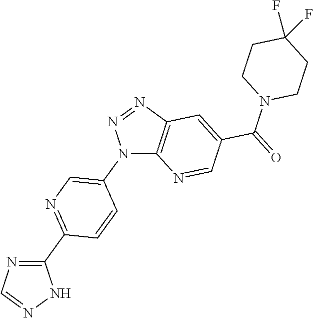 [SMILES: O=C(c1cnc2c(c1)nnn2-c1ccc(-c2ncn[nH]2)nc1)N1CCC(F)(F)CC1] |
| C-6 | 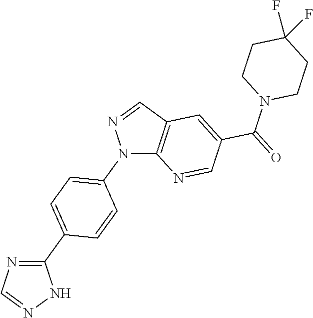 [SMILES: O=C(c1cnc2c(cnn2-c2ccc(-c3ncn[nH]3)cc2)c1)N1CCC(F)(F)CC1] |
| C-7 | 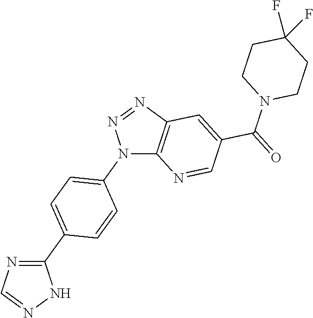 [SMILES: O=C(c1cnc2c(c1)nnn2-c1ccc(-c2ncn[nH]2)cc1)N1CCC(F)(F)CC1] |
| C-8 | 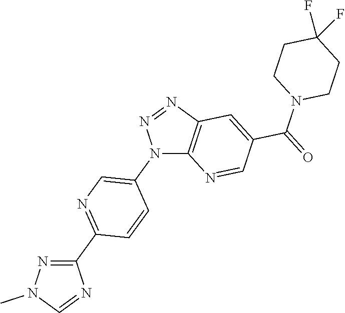 [SMILES: Cn1cnc(-c2ccc(-n3nnc4cc(C(=O)N5CCC(F)(F)CC5)cnc43)cn2)n1] |
| C-9 | 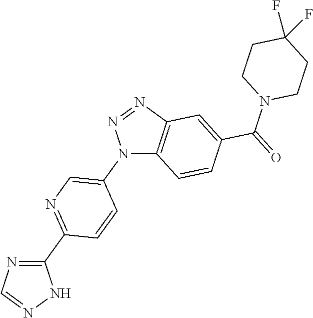 [SMILES: O=C(c1ccc2c(c1)nnn2-c1ccc(-c2ncn[nH]2)nc1)N1CCC(F)(F)CC1] |
| C-10 | 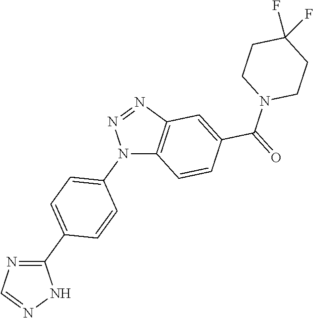 [SMILES: O=C(c1ccc2c(c1)nnn2-c1ccc(-c2ncn[nH]2)cc1)N1CCC(F)(F)CC1] |
| C-11 | 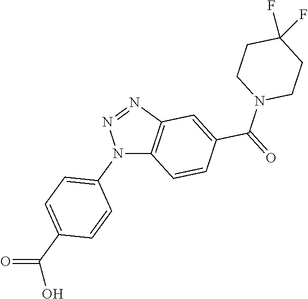 [SMILES: O=C(O)c1ccc(-n2nnc3cc(C(=O)N4CCC(F)(F)CC4)ccc32)cc1] |
| C-12 | 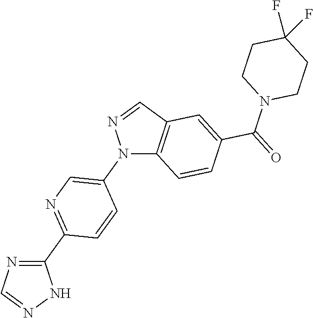 [SMILES: O=C(c1ccc2c(cnn2-c2ccc(-c3ncn[nH]3)nc2)c1)N1CCC(F)(F)CC1] |
| C-13 | 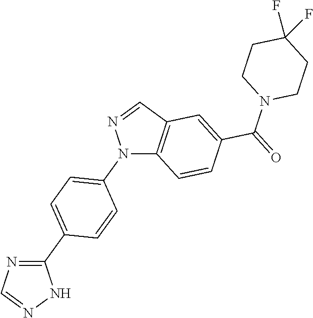 [SMILES: O=C(c1ccc2c(cnn2-c2ccc(-c3ncn[nH]3)cc2)c1)N1CCC(F)(F)CC1] |
| C-14 | 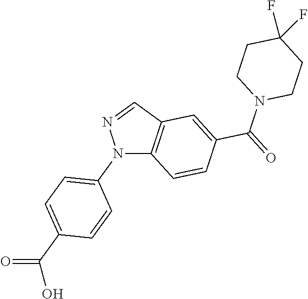 [SMILES: O=C(O)c1ccc(-n2ncc3cc(C(=O)N4CCC(F)(F)CC4)ccc32)cc1] |
| C-15 | 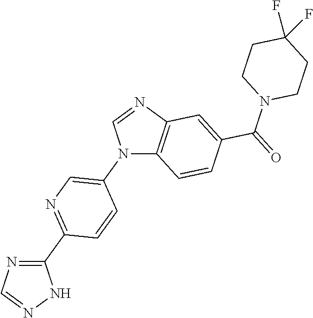 [SMILES: O=C(c1ccc2c(c1)ncn2-c1ccc(-c2ncn[nH]2)nc1)N1CCC(F)(F)CC1] |
| C-16 | 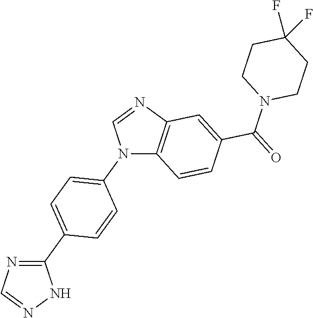 [SMILES: O=C(c1ccc2c(c1)ncn2-c1ccc(-c2ncn[nH]2)cc1)N1CCC(F)(F)CC1] |
| C-17 | 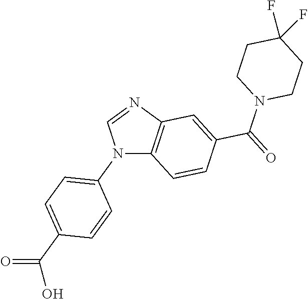 [SMILES: O=C(O)c1ccc(-n2cnc3cc(C(=O)N4CCC(F)(F)CC4)ccc32)cc1] |
| C-18 | 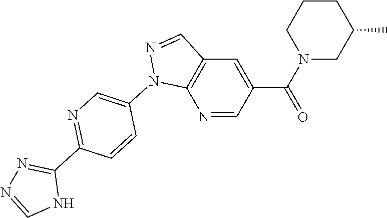 [SMILES: C[C@H]1CCCN(C(=O)c2cnc3c(cnn3-c3ccc(-c4nnc[nH]4)nc3)c2)C1] |
| C-19 | 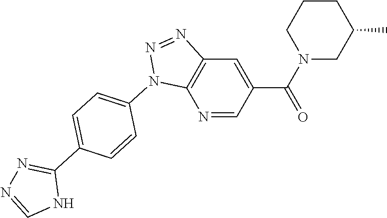 [SMILES: C[C@H]1CCCN(C(=O)c2cnc3c(c2)nnn3-c2ccc(-c3nnc[nH]3)cc2)C1] |
| C-20 | 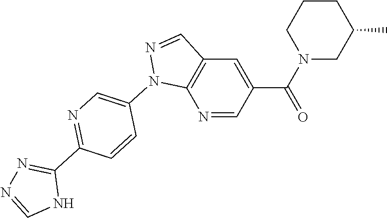 [SMILES: C[C@H]1CCCN(C(=O)c2cnc3c(cnn3-c3ccc(-c4nnc[nH]4)nc3)c2)C1] |
| C-21 | 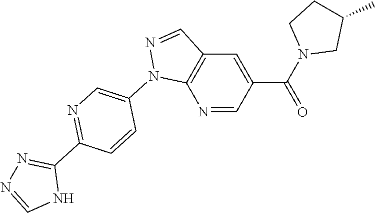 [SMILES: C[C@H]1CCN(C(=O)c2cnc3c(cnn3-c3ccc(-c4nnc[nH]4)nc3)c2)C1] |
| C-22 | 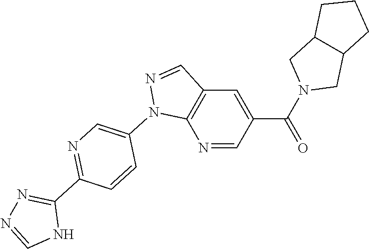 [SMILES: O=C(c1cnc2c(cnn2-c2ccc(-c3nnc[nH]3)nc2)c1)N1CC2CCCC2C1] |
| C-23 | 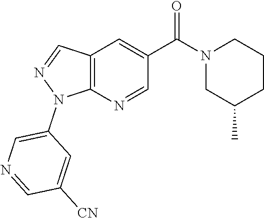 [SMILES: C[C@H]1CCCN(C(=O)c2cnc3c(cnn3-c3cncc(C#N)c3)c2)C1] |
| C-24 | 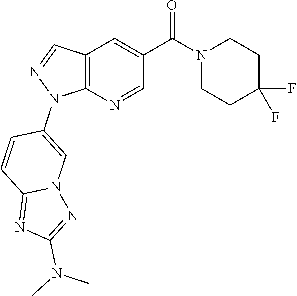 [SMILES: CN(C)c1nc2ccc(-n3ncc4cc(C(=O)N5CCC(F)(F)CC5)cnc43)cn2n1] |
| C-25 | 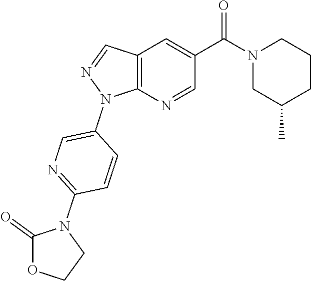 [SMILES: C[C@H]1CCCN(C(=O)c2cnc3c(cnn3-c3ccc(N4CCOC4=O)nc3)c2)C1] |
| C-26 | 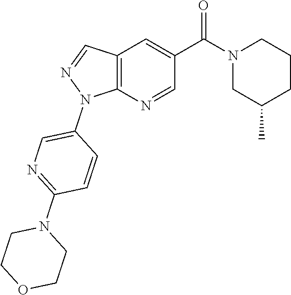 [SMILES: C[C@H]1CCCN(C(=O)c2cnc3c(cnn3-c3ccc(N4CCOCC4)nc3)c2)C1] |
| C-27 | 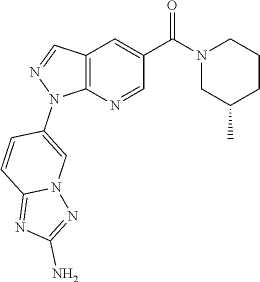 [SMILES: C[C@H]1CCCN(C(=O)c2cnc3c(cnn3-c3ccc4nc(N)nn4c3)c2)C1] |
| C-28 | 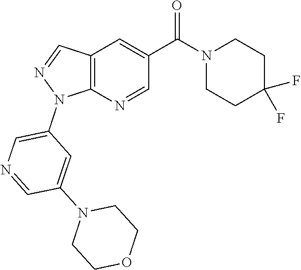 [SMILES: O=C(c1cnc2c(cnn2-c2cncc(N3CCOCC3)c2)c1)N1CCC(F)(F)CC1] |
| C-29 | 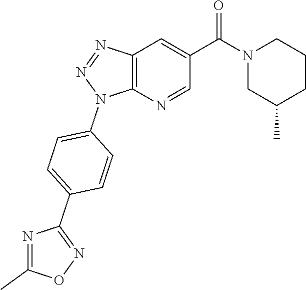 [SMILES: Cc1nc(-c2ccc(-n3nnc4cc(C(=O)N5CCC[C@H](C)C5)cnc43)cc2)no1] |
| C-30 | 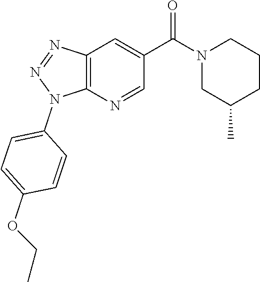 [SMILES: CCOc1ccc(-n2nnc3cc(C(=O)N4CCC[C@H](C)C4)cnc32)cc1] |
| C-31 | 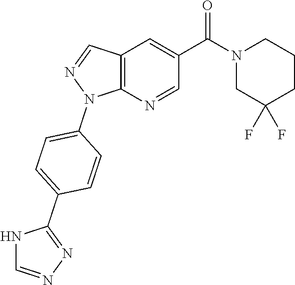 [SMILES: O=C(c1cnc2c(cnn2-c2ccc(-c3nnc[nH]3)cc2)c1)N1CCCC(F)(F)C1] |
| C-32 | 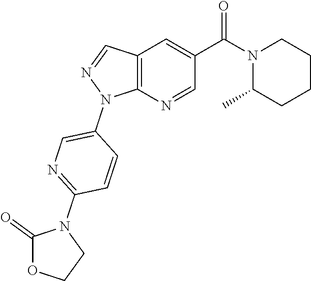 [SMILES: C[C@H]1CCCCN1C(=O)c1cnc2c(cnn2-c2ccc(N3CCOC3=O)nc2)c1] |
| C-33 | 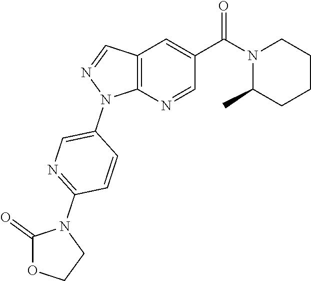 [SMILES: C[C@@H]1CCCCN1C(=O)c1cnc2c(cnn2-c2ccc(N3CCOC3=O)nc2)c1] |
| C-34 | 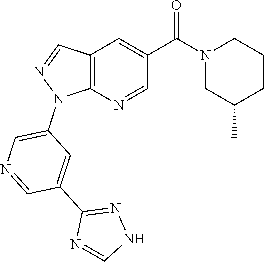 [SMILES: C[C@H]1CCCN(C(=O)c2cnc3c(cnn3-c3cncc(-c4nc[nH]n4)c3)c2)C1] |
| C-35 | 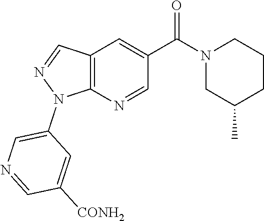 [SMILES: C[C@H]1CCCN(C(=O)c2cnc3c(cnn3-c3cncc(C(N)=O)c3)c2)C1] |
| C-36 | 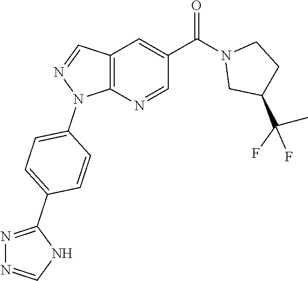 [SMILES: CC(F)(F)[C@@H]1CCN(C(=O)c2cnc3c(cnn3-c3ccc(-c4nnc[nH]4)cc3)c2)C1] |
| C-37 | 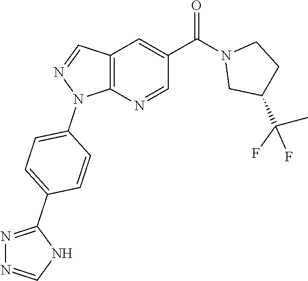 [SMILES: CC(F)(F)[C@H]1CCN(C(=O)c2cnc3c(cnn3-c3ccc(-c4nnc[nH]4)cc3)c2)C1] |
| C-38 | 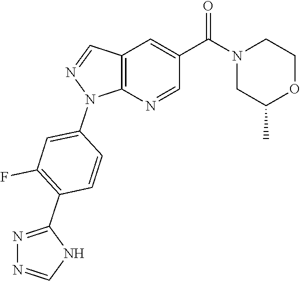 [SMILES: C[C@@H]1CN(C(=O)c2cnc3c(cnn3-c3ccc(-c4nnc[nH]4)c(F)c3)c2)CCO1] |
| C-39 | 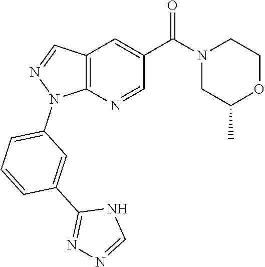 [SMILES: C[C@@H]1CN(C(=O)c2cnc3c(cnn3-c3cccc(-c4nnc[nH]4)c3)c2)CCO1] |
| C-40 | 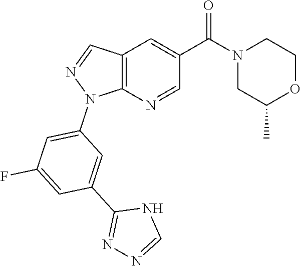 [SMILES: C[C@@H]1CN(C(=O)c2cnc3c(cnn3-c3cc(F)cc(-c4nnc[nH]4)c3)c2)CCO1] |
| C-41 | 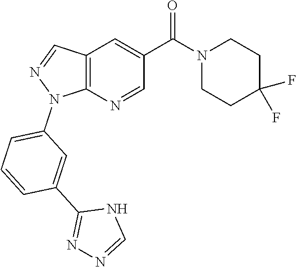 [SMILES: O=C(c1cnc2c(cnn2-c2cccc(-c3nnc[nH]3)c2)c1)N1CCC(F)(F)CC1] |
| C-42 | 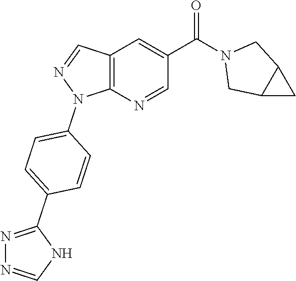 [SMILES: O=C(c1cnc2c(cnn2-c2ccc(-c3nnc[nH]3)cc2)c1)N1CC2CC2C1] |
| C-43 | 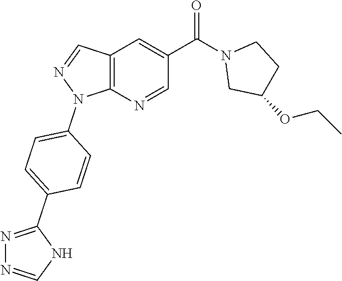 [SMILES: CCO[C@H]1CCN(C(=O)c2cnc3c(cnn3-c3ccc(-c4nnc[nH]4)cc3)c2)C1] |
| C-44 | 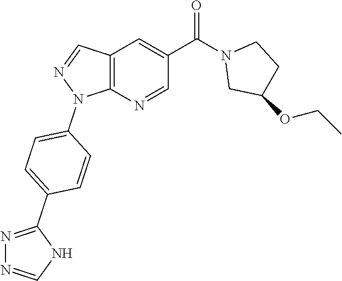 [SMILES: CCO[C@@H]1CCN(C(=O)c2cnc3c(cnn3-c3ccc(-c4nnc[nH]4)cc3)c2)C1] |
| C-45 | 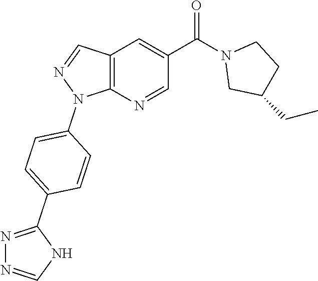 [SMILES: CC[C@H]1CCN(C(=O)c2cnc3c(cnn3-c3ccc(-c4nnc[nH]4)cc3)c2)C1] |
| C-46 | 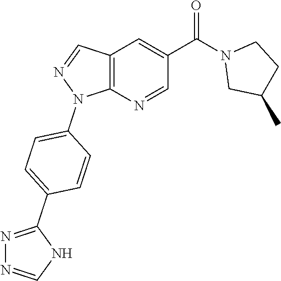 [SMILES: C[C@@H]1CCN(C(=O)c2cnc3c(cnn3-c3ccc(-c4nnc[nH]4)cc3)c2)C1] |
| C-47 | 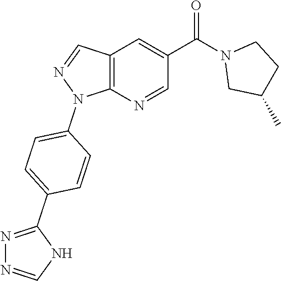 [SMILES: C[C@H]1CCN(C(=O)c2cnc3c(cnn3-c3ccc(-c4nnc[nH]4)cc3)c2)C1] |
| C-48 | 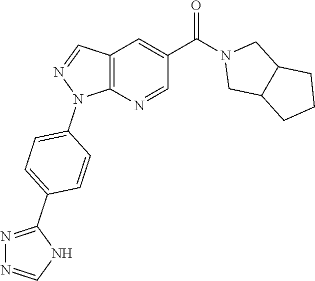 [SMILES: O=C(c1cnc2c(cnn2-c2ccc(-c3nnc[nH]3)cc2)c1)N1CC2CCCC2C1] |
| C-49 | 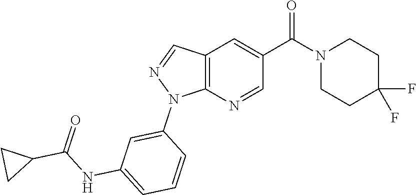 [SMILES: O=C(Nc1cccc(-n2ncc3cc(C(=O)N4CCC(F)(F)CC4)cnc32)c1)C1CC1] |
| C-50 | 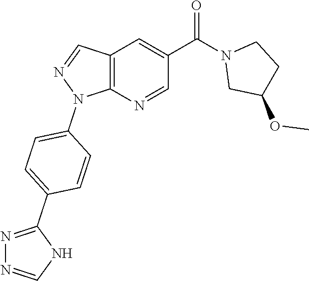 [SMILES: CO[C@@H]1CCN(C(=O)c2cnc3c(cnn3-c3ccc(-c4nnc[nH]4)cc3)c2)C1] |
| C-51 | 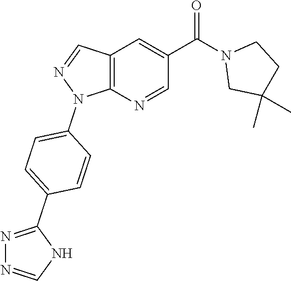 [SMILES: CC1(C)CCN(C(=O)c2cnc3c(cnn3-c3ccc(-c4nnc[nH]4)cc3)c2)C1] |
| C-52 | 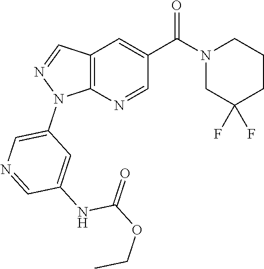 [SMILES: CCOC(=O)Nc1cncc(-n2ncc3cc(C(=O)N4CCCC(F)(F)C4)cnc32)c1] |
| C-53 | 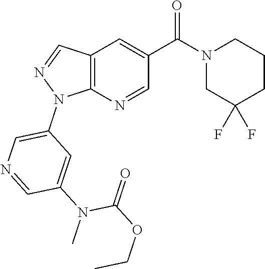 [SMILES: CCOC(=O)N(C)c1cncc(-n2ncc3cc(C(=O)N4CCCC(F)(F)C4)cnc32)c1] |
| C-54 | 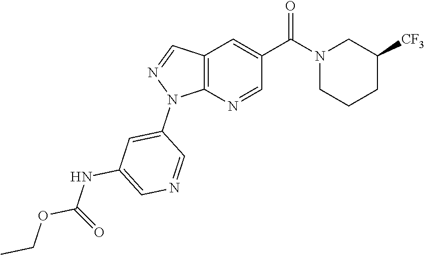 [SMILES: CCOC(=O)Nc1cncc(-n2ncc3cc(C(=O)N4CCC[C@H](C(F)(F)F)C4)cnc32)c1] |
| C-55 | 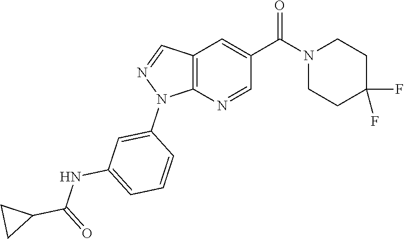 [SMILES: O=C(Nc1cccc(-n2ncc3cc(C(=O)N4CCC(F)(F)CC4)cnc32)c1)C1CC1] |
| C-56 | 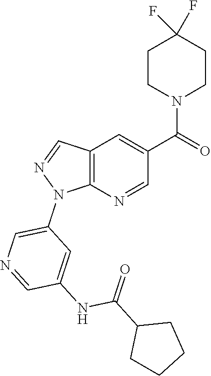 [SMILES: O=C(Nc1cncc(-n2ncc3cc(C(=O)N4CCC(F)(F)CC4)cnc32)c1)C1CCCC1] |
| C-57 | 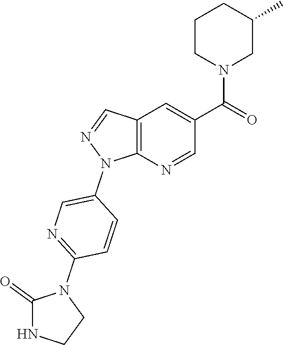 [SMILES: C[C@H]1CCCN(C(=O)c2cnc3c(cnn3-c3ccc(N4CCNC4=O)nc3)c2)C1] |
| C-58 | 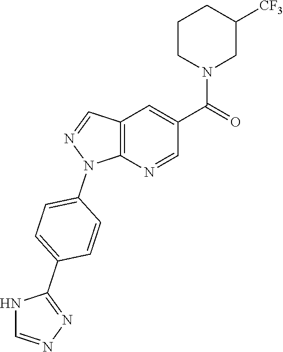 [SMILES: O=C(c1cnc2c(cnn2-c2ccc(-c3nnc[nH]3)cc2)c1)N1CCCC(C(F)(F)F)C1] |
| C-59 | 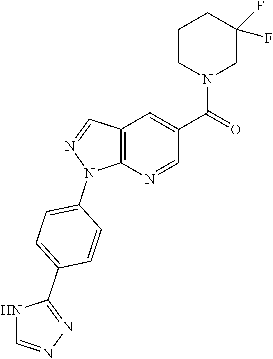 [SMILES: O=C(c1cnc2c(cnn2-c2ccc(-c3nnc[nH]3)cc2)c1)N1CCCC(F)(F)C1] |
| C-60 | 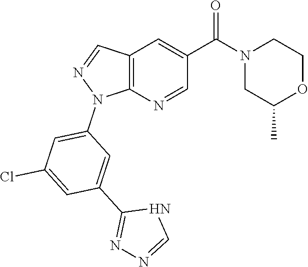 [SMILES: C[C@@H]1CN(C(=O)c2cnc3c(cnn3-c3cc(Cl)cc(-c4nnc[nH]4)c3)c2)CCO1] |
| C-61 | 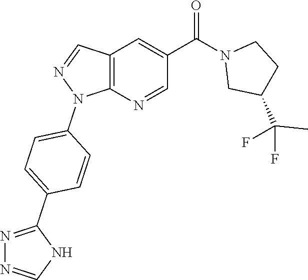 [SMILES: CC(F)(F)[C@H]1CCN(C(=O)c2cnc3c(cnn3-c3ccc(-c4nnc[nH]4)cc3)c2)C1] |
| C-62 | 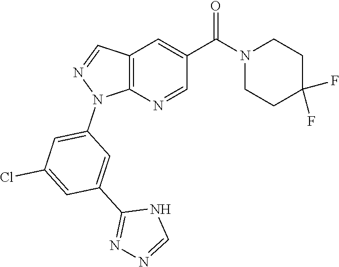 [SMILES: O=C(c1cnc2c(cnn2-c2cc(Cl)cc(-c3nnc[nH]3)c2)c1)N1CCC(F)(F)CC1] |
| C-63 | 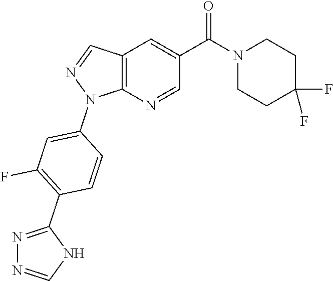 [SMILES: O=C(c1cnc2c(cnn2-c2ccc(-c3nnc[nH]3)c(F)c2)c1)N1CCC(F)(F)CC1] |
| C-64 | 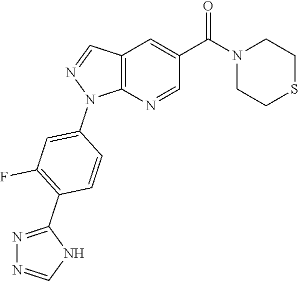 [SMILES: O=C(c1cnc2c(cnn2-c2ccc(-c3nnc[nH]3)c(F)c2)c1)N1CCSCC1] |
| C-65 | 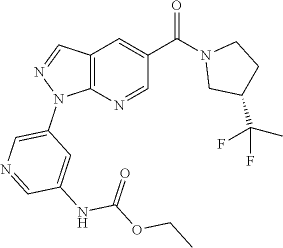 [SMILES: CCOC(=O)Nc1cncc(-n2ncc3cc(C(=O)N4CC[C@H](C(C)(F)F)C4)cnc32)c1] |
| C-66 | 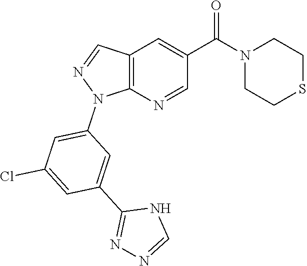 [SMILES: O=C(c1cnc2c(cnn2-c2cc(Cl)cc(-c3nnc[nH]3)c2)c1)N1CCSCC1] |
| C-67 | 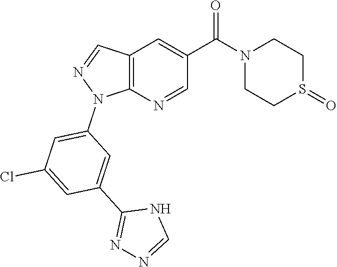 [SMILES: O=C(c1cnc2c(cnn2-c2cc(Cl)cc(-c3nnc[nH]3)c2)c1)N1CCS(=O)CC1] |
| C-68 | 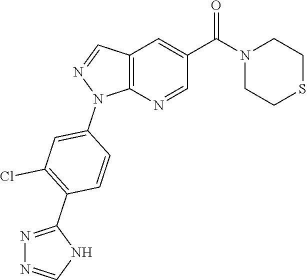 [SMILES: O=C(c1cnc2c(cnn2-c2ccc(-c3nnc[nH]3)c(Cl)c2)c1)N1CCSCC1] |
| C-69 | 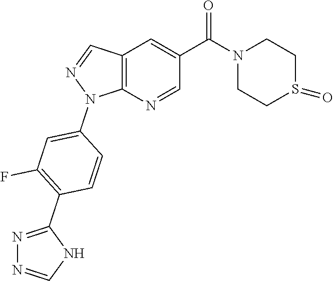 [SMILES: O=C(c1cnc2c(cnn2-c2ccc(-c3nnc[nH]3)c(F)c2)c1)N1CCS(=O)CC1] |
| C-70 | 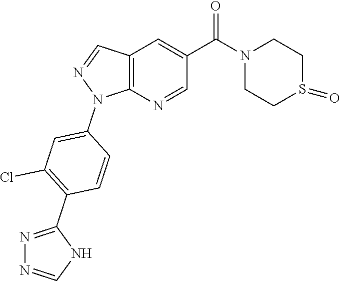 [SMILES: O=C(c1cnc2c(cnn2-c2ccc(-c3nnc[nH]3)c(Cl)c2)c1)N1CCS(=O)CC1] |
| C-71 | 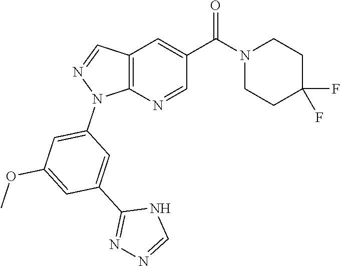 [SMILES: COc1cc(-c2nnc[nH]2)cc(-n2ncc3cc(C(=O)N4CCC(F)(F)CC4)cnc32)c1] |
| C-72 | 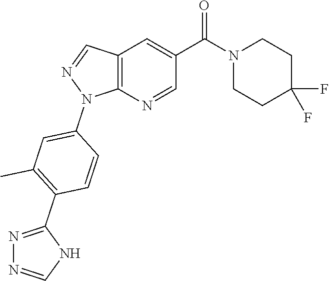 [SMILES: Cc1cc(-n2ncc3cc(C(=O)N4CCC(F)(F)CC4)cnc32)ccc1-c1nnc[nH]1] |
| C-73 | 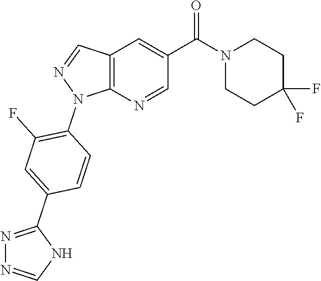 [SMILES: O=C(c1cnc2c(cnn2-c2ccc(-c3nnc[nH]3)cc2F)c1)N1CCC(F)(F)CC1] |
| C-74 | 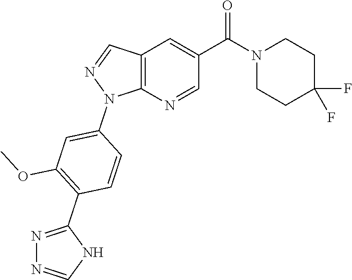 [SMILES: COc1cc(-n2ncc3cc(C(=O)N4CCC(F)(F)CC4)cnc32)ccc1-c1nnc[nH]1] |
| C-75 | 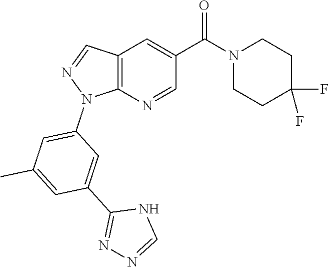 [SMILES: Cc1cc(-c2nnc[nH]2)cc(-n2ncc3cc(C(=O)N4CCC(F)(F)CC4)cnc32)c1] |
| C-76 | 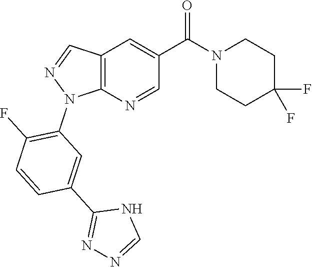 [SMILES: O=C(c1cnc2c(cnn2-c2cc(-c3nnc[nH]3)ccc2F)c1)N1CCC(F)(F)CC1] |
| C-77 | 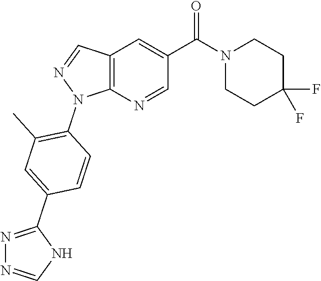 [SMILES: Cc1cc(-c2nnc[nH]2)ccc1-n1ncc2cc(C(=O)N3CCC(F)(F)CC3)cnc21] |
| C-78 | 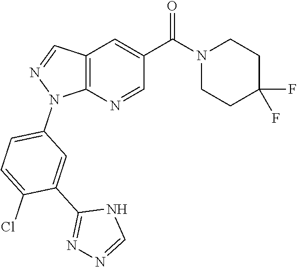 [SMILES: O=C(c1cnc2c(cnn2-c2ccc(Cl)c(-c3nnc[nH]3)c2)c1)N1CCC(F)(F)CC1] |
| C-79 | 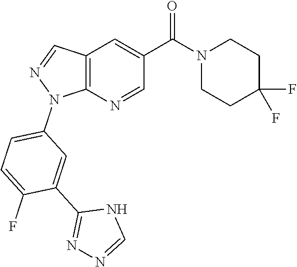 [SMILES: O=C(c1cnc2c(cnn2-c2ccc(F)c(-c3nnc[nH]3)c2)c1)N1CCC(F)(F)CC1] |
| C-80 | 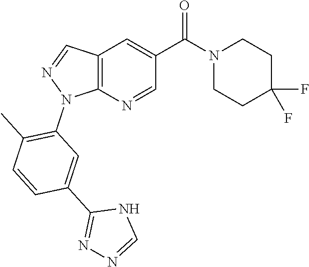 [SMILES: Cc1ccc(-c2nnc[nH]2)cc1-n1ncc2cc(C(=O)N3CCC(F)(F)CC3)cnc21] |
| C-81 | 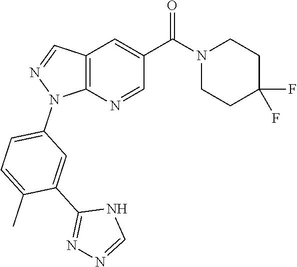 [SMILES: Cc1ccc(-n2ncc3cc(C(=O)N4CCC(F)(F)CC4)cnc32)cc1-c1nnc[nH]1] |
| C-82 | 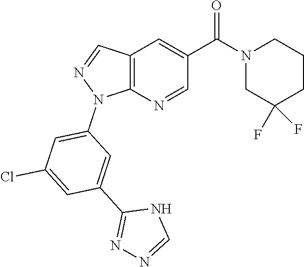 [SMILES: O=C(c1cnc2c(cnn2-c2cc(Cl)cc(-c3nnc[nH]3)c2)c1)N1CCCC(F)(F)C1] |
| C-83 | 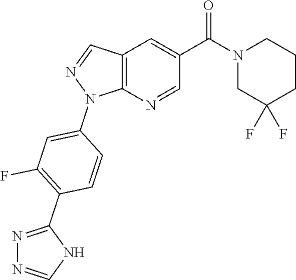 [SMILES: O=C(c1cnc2c(cnn2-c2ccc(-c3nnc[nH]3)c(F)c2)c1)N1CCCC(F)(F)C1] |
| C-84 | 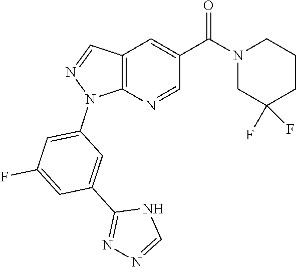 [SMILES: O=C(c1cnc2c(cnn2-c2cc(F)cc(-c3nnc[nH]3)c2)c1)N1CCCC(F)(F)C1] |
| C-85 | 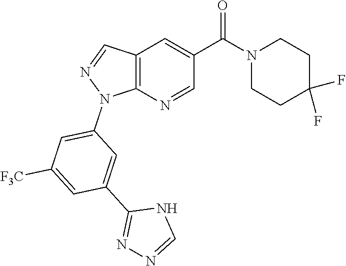 [SMILES: O=C(c1cnc2c(cnn2-c2cc(-c3nnc[nH]3)cc(C(F)(F)F)c2)c1)N1CCC(F)(F)CC1] |

In some embodiments, the PGDH inhibitor is a compound provided in Table 1b, or a pharmaceutically acceptable salt or solvate thereof.

TABLE 1b
Representative sulfoxide PGDH inhibitors

| Compound No | Structure |
|---|---|
| C-86 | 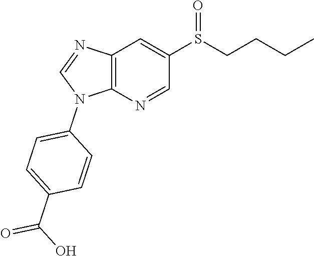 [SMILES: CCCCS(=O)c1cnc2c(c1)ncn2-c1ccc(C(=O)O)cc1] |
| C-87 | 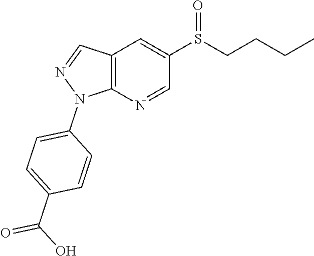 [SMILES: CCCCS(=O)c1cnc2c(cnn2-c2ccc(C(=O)O)cc2)c1] |
| C-88 | 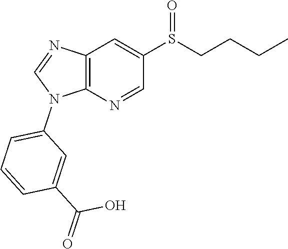 [SMILES: CCCCS(=O)c1cnc2c(c1)ncn2-c1cccc(C(=O)O)c1] |
| C-89 | 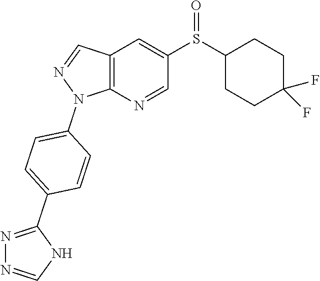 [SMILES: O=S(c1cnc2c(cnn2-c2ccc(-c3nnc[nH]3)cc2)c1)C1CCC(F)(F)CC1] |
| C-90 | 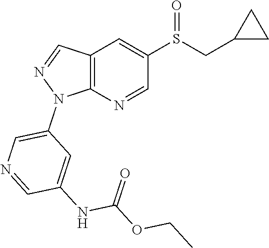 [SMILES: CCOC(=O)Nc1cncc(-n2ncc3cc(S(=O)CC4CC4)cnc32)c1] |
| C-91 | 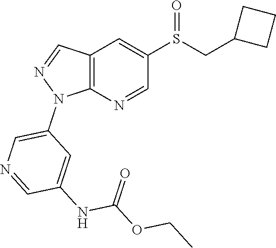 [SMILES: CCOC(=O)Nc1cncc(-n2ncc3cc(S(=O)CC4CCC4)cnc32)c1] |
| C-92 | 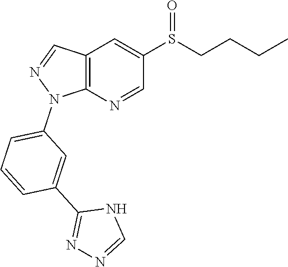 [SMILES: CCCCS(=O)c1cnc2c(cnn2-c2cccc(-c3nnc[nH]3)c2)c1] |
| C-93 | 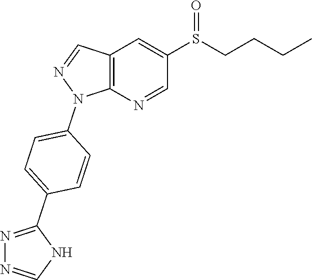 [SMILES: CCCCS(=O)c1cnc2c(cnn2-c2ccc(-c3nnc[nH]3)cc2)c1] |
| C-94 | 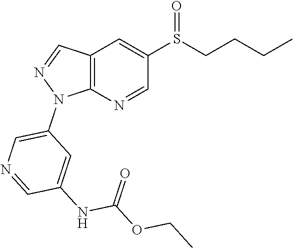 [SMILES: CCCCS(=O)c1cnc2c(cnn2-c2cncc(NC(=O)OCC)c2)c1] |
| C-95 | 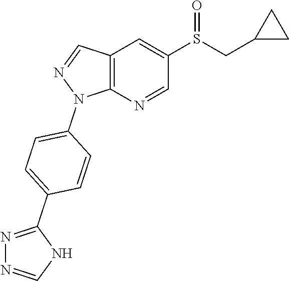 [SMILES: O=S(CC1CC1)c1cnc2c(cnn2-c2ccc(-c3nnc[nH]3)cc2)c1] |
| C-96 | 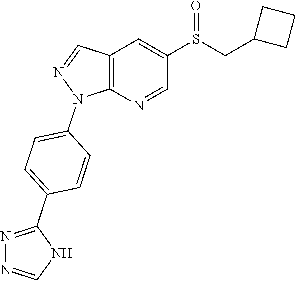 [SMILES: O=S(CC1CCC1)c1cnc2c(cnn2-c2ccc(-c3nnc[nH]3)cc2)c1] |
| C-97 | 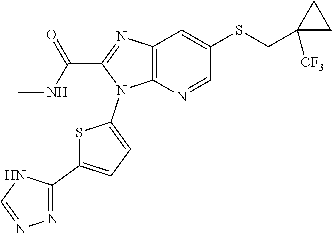 [SMILES: CNC(=O)c1nc2cc(SCC3(C(F)(F)F)CC3)cnc2n1-c1ccc(-c2nnc[nH]2)s1] |
| C-98 | 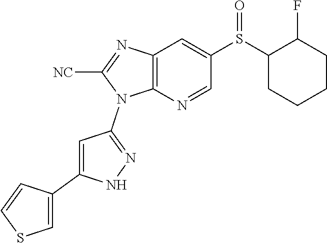 [SMILES: N#Cc1nc2cc(S(=O)C3CCCCC3F)cnc2n1-c1cc(-c2ccsc2)[nH]n1] |
| C-99 | 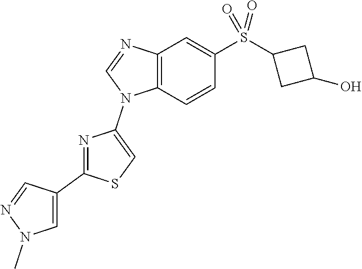 [SMILES: Cn1cc(-c2nc(-n3cnc4cc(S(=O)(=O)C5CC(O)C5)ccc43)cs2)cn1] |
| C-100 | 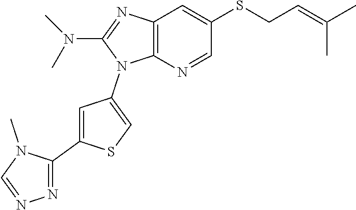 [SMILES: CC(C)=CCSc1cnc2c(c1)nc(N(C)C)n2-c1csc(-c2nncn2C)c1] |
| C-101 | 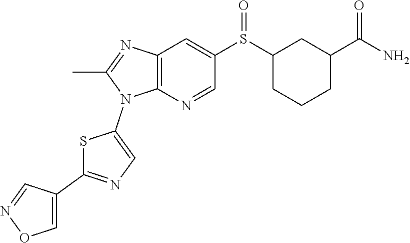 [SMILES: Cc1nc2cc(S(=O)C3CCCC(C(N)=O)C3)cnc2n1-c1cnc(-c2cnoc2)s1] |
| C-102 | 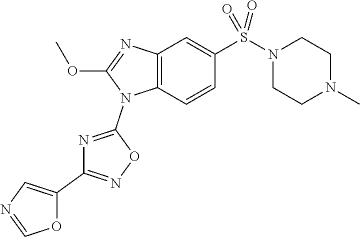 [SMILES: COc1nc2cc(S(=O)(=O)N3CCN(C)CC3)ccc2n1-c1nc(-c2cnco2)no1] |
| C-103 | 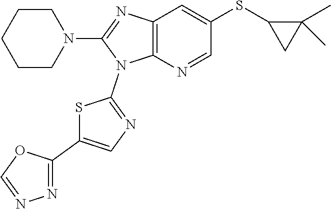 [SMILES: CC1(C)CC1Sc1cnc2c(c1)nc(N1CCCCC1)n2-c1ncc(-c2nnco2)s1] |
| C-104 | 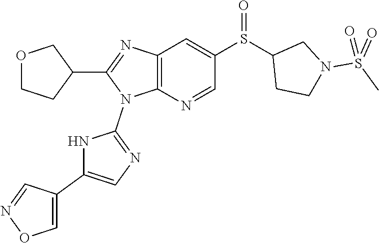 [SMILES: CS(=O)(=O)N1CCC(S(=O)c2cnc3c(c2)nc(C2CCOC2)n3-c2ncc(-c3cnoc3)[nH]2)C1] |
| C-105 | 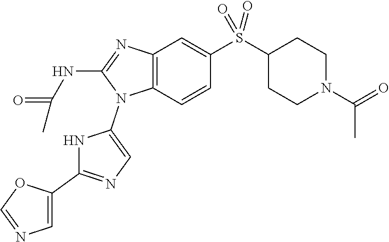 [SMILES: CC(=O)Nc1nc2cc(S(=O)(=O)C3CCN(C(C)=O)CC3)ccc2n1-c1cnc(-c2cnco2)[nH]1] |
| C-106 | 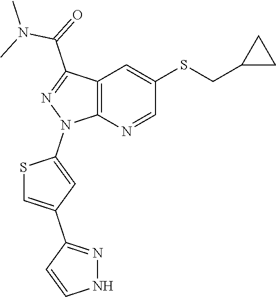 [SMILES: CN(C)C(=O)c1nn(-c2cc(-c3cc[nH]n3)cs2)c2ncc(SCC3CC3)cc12] |
| C-107 | 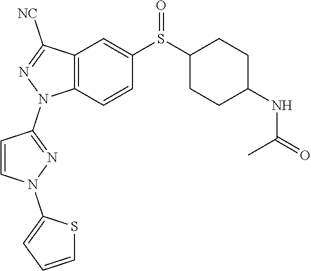 [SMILES: CC(=O)NC1CCC(S(=O)c2ccc3c(c2)c(C#N)nn3-c2ccn(-c3cccs3)n2)CC1] |
| C-108 | 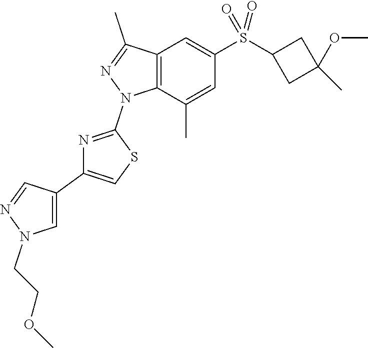 [SMILES: COCCn1cc(-c2csc(-n3nc(C)c4cc(S(=O)(=O)C5CC(C)(OC)C5)cc(C)c43)n2)cn1] |
| C-109 | 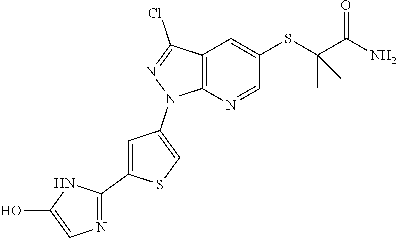 [SMILES: CC(C)(Sc1cnc2c(c1)c(Cl)nn2-c1csc(-c2ncc(O)[nH]2)c1)C(N)=O] |
| C-110 | 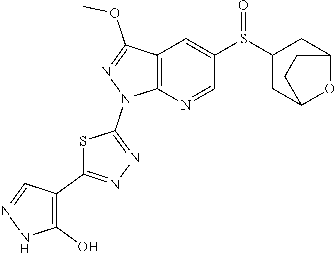 [SMILES: COc1nn(-c2nnc(-c3cn[nH]c3O)s2)c2ncc(S(=O)C3CC4CCC(C3)O4)cc12] |
| C-111 | 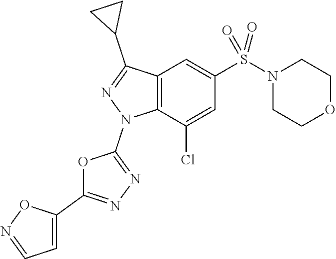 [SMILES: O=S(=O)(c1cc(Cl)c2c(c1)c(C1CC1)nn2-c1nnc(-c2ccno2)o1)N1CCOCC1] |
| C-112 | 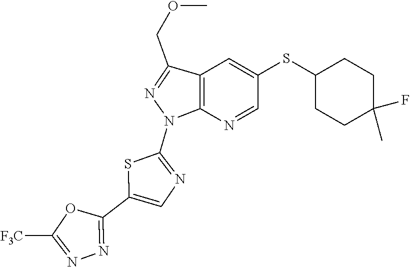 |
| C-113 | 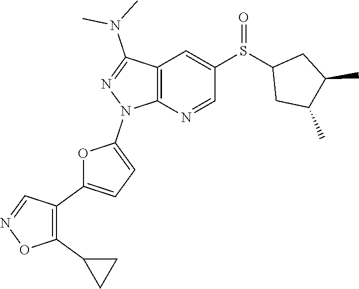 [SMILES: C[C@@H]1CC(S(=O)c2cnc3c(c2)c(N(C)C)nn3-c2ccc(-c3cnoc3C3CC3)o2)C[C@H]1C] |
| C-114 | 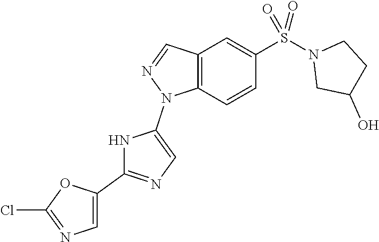 [SMILES: O=S(=O)(c1ccc2c(cnn2-c2cnc(-c3cnc(Cl)o3)[nH]2)c1)N1CCC(O)C1] |
| C-115 | 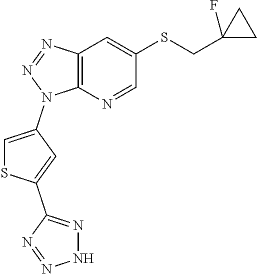 [SMILES: FC1(CSc2cnc3c(c2)nnn3-c2csc(-c3nn[nH]n3)c2)CC1] |
| C-116 | 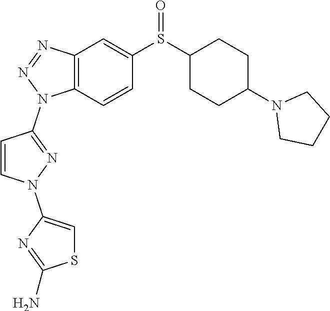 [SMILES: Nc1nc(-n2ccc(-n3nnc4cc(S(=O)C5CCC(N6CCCC6)CC5)ccc43)n2)cs1] |
| C-117 | 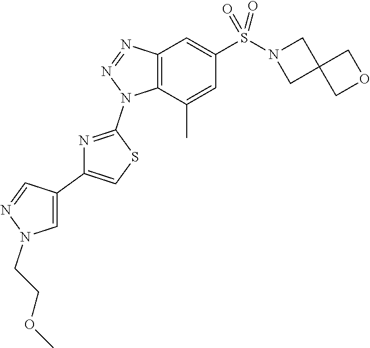 [SMILES: COCCn1cc(-c2csc(-n3nnc4cc(S(=O)(=O)N5CC6(COC6)C5)cc(C)c43)n2)cn1] |
| C-118 | 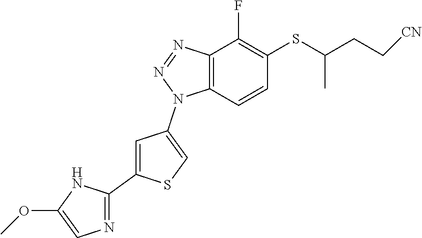 [SMILES: COc1cnc(-c2cc(-n3nnc4c(F)c(SC(C)CCC#N)ccc43)cs2)[nH]1] |
| C-119 | 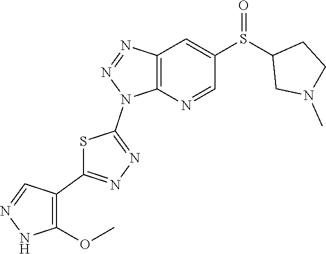 [SMILES: COc1[nH]ncc1-c1nnc(-n2nnc3cc(S(=O)C4CCN(C)C4)cnc32)s1] |
| C-120 | 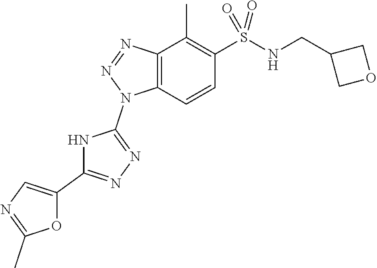 [SMILES: Cc1ncc(-c2nnc(-n3nnc4c(C)c(S(=O)(=O)NCC5COC5)ccc43)[nH]2)o1] |
| C-121 | 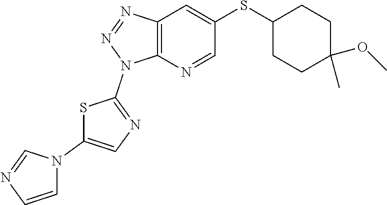 [SMILES: COC1(C)CCC(Sc2cnc3c(c2)nnn3-c2ncc(-n3ccnc3)s2)CC1] |
| C-122 | 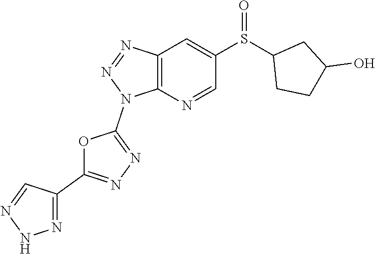 [SMILES: O=S(c1cnc2c(c1)nnn2-c1nnc(-c2cn[nH]n2)o1)C1CCC(O)C1] |
| C-123 | 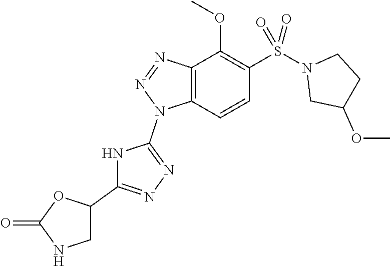 [SMILES: COc1c(S(=O)(=O)N2CCC(OC)C2)ccc2c1nnn2-c1nnc(C2CNC(=O)O2)[nH]1] |
| C-124 | 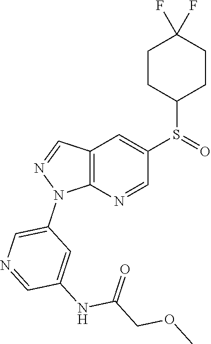 [SMILES: COCC(=O)Nc1cncc(-n2ncc3cc(S(=O)C4CCC(F)(F)CC4)cnc32)c1] |
| C-125 | 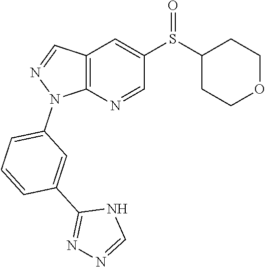 [SMILES: O=S(c1cnc2c(cnn2-c2cccc(-c3nnc[nH]3)c2)c1)C1CCOCC1] |

In some embodiments, the PGDH inhibitor is a compound provided in Table 1c, or a pharmaceutically acceptable salt or solvate thereof.

TABLE 1c
Representative PGDH inhibitors.

| Compound | |
|---|---|
| No | Structure |
| D-1 | 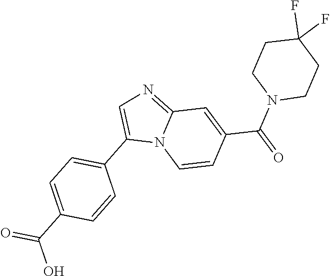 [SMILES: O=C(O)c1ccc(-c2cnc3cc(C(=O)N4CCC(F)(F)CC4)ccn23)cc1] |
| D2 | 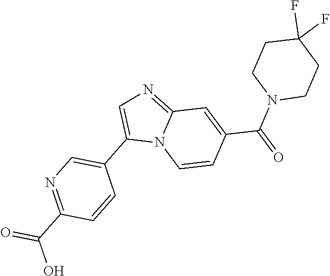 [SMILES: O=C(O)c1ccc(-c2cnc3cc(C(=O)N4CCC(F)(F)CC4)ccn23)cn1] |
| D-3 | 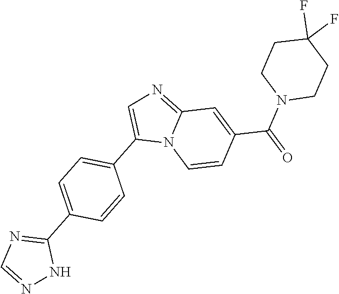 [SMILES: O=C(c1ccn2c(-c3ccc(-c4ncn[nH]4)cc3)cnc2c1)N1CCC(F)(F)CC1] |
| D-4 | 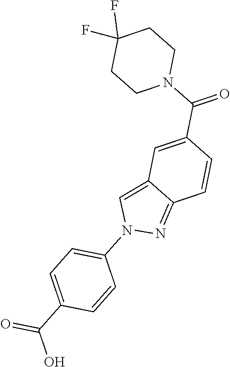 [SMILES: O=C(O)c1ccc(-n2cc3cc(C(=O)N4CCC(F)(F)CC4)ccc3n2)cc1] |

### Methods of Use

In one aspect, provided herein are methods for treating various disorders in a subject in need thereof, comprising administering to said subject a compound described herein. In some embodiments, the inhibitors of hydroxyprostaglandin dehydrogenase provided herein may be used for the prevention or treatment of a disease or a disorder that is associated with hydroxyprostaglandin dehydrogenase (such as 15-PGDH) and/or decreased levels of prostaglandins. In some embodiments, the inhibitors of hydroxyprostaglandin dehydrogenase provided herein may be used for the prevention or treatment of a disease or a disorder in which it is desirable to increase prostaglandin levels in the subject having said disease or disorder.

In some embodiments, the methods for treating the disorders comprises administering to said subject a 15-PGDH inhibitor. In some embodiments, a compound described herein is the 15-PGDH inhibitor (e.g. a compound of Formula (V) or a pharmaceutically acceptable salt or solvate thereof). In some embodiments, the methods comprise administering a therapeutically effective amount of a compound described herein. In some embodiments, the methods comprise administering a therapeutically effective amount of a compound described herein or a pharmaceutically acceptable salt or solvate thereof (e.g. a compound of Formula (V) or a pharmaceutically acceptable salt or solvate thereof). In some embodiments, the compound described herein is a 15-PGDH inhibitor (e.g. a compound of Formula (V), or a pharmaceutically acceptable salt or solvate thereof). In some embodiments, the administration takes place in vitro. In other embodiments, the administration takes place in vivo.

As used herein, a therapeutically effective amount of a 15-PGDH inhibitor refers to an amount sufficient to effect the intended application, including but not limited to, disease treatment, as defined herein. Also contemplated in the subject methods is the use of a sub-therapeutic amount of a 15-PGDH inhibitor for treating an intended disease condition.

The amount of the 15-PGDH inhibitor administered may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

Measuring inhibition of biological effects of 15-PGDH can comprise performing an assay on a biological sample, such as a sample from a subject. Any of a variety of samples may be selected, depending on the assay. Examples of samples include, but are not limited to, blood samples (e.g. blood plasma or serum), exhaled breath condensate samples, bronchoalveolar lavage fluid, sputum samples, urine samples, and tissue samples.

A subject being treated with a 15-PGDH inhibitor may be monitored to determine the effectiveness of treatment, and the treatment regimen may be adjusted based on the subject's physiological response to treatment. For example, if inhibition of a biological effect of 15-PGDH is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with a 15-PGDH inhibitor is discontinued if inhibition of the biological effect is above or below a threshold, such as in a lack of response or an adverse reaction. The biological effect may be a change in any of a variety of physiological indicators.

In general, a 15-PGDH inhibitor is a compound that inhibits one or more biological effects of 15-PGDH. Such biological effects may be inhibited by about or more than about 10%, 20%, 30%, 40%, 50%, 60%, 70%, 80%, 90%, 95%, or more.

In some other embodiments, the subject methods are useful for treating a disease condition associated with 15-PGDH. Any disease condition that results directly or indirectly from an abnormal activity or expression level of 15-PGDH can be an intended disease condition.

In one aspect, provided herein is a method of promoting and/or stimulation skin pigmentation, comprising administering one or more of the compositions described herein to a subject in need thereof. Inhibitors of 15-PGDH are known to promote skin pigmentation (Markowitz et. al., WO 2015/065716). The hydroxyprostaglandin dehydrogenase inhibitors described herein can be used for promoting and/or inducing and/or stimulating pigmentation of the skin and/or skin appendages, and/or as an agent for preventing and/or limiting depigmentation and/or whitening of the skin and/or skin appendages, in particular as an agent for preventing and/or limiting canities. In some embodiments, the 15-PGDH inhibitors provided herein can be applied to skin of a subject, e.g., in a topical application, to promote and/or stimulate pigmentation of the skin and/or hair growth, inhibit hair loss, and/or treat skin damage or inflammation, such as skin damage caused by physical or chemical irritants and/or UV-exposure.

In another aspect, provided herein is a method of inhibiting hair loss, comprising administering one or more of the compositions described herein to a subject in need thereof. It is known that prostaglandins play an important role in hair growth. Prostaglandins such as prostaglandin A1, F2a and E2 are stored in hair follicles or adjacent skin environments and have been shown to be essential in maintaining and increasing hair density (Colombe L et. al, 2007, Exp. Dermatol, 16(9), 762-9). It has been reported that 15-PGDH, which is involved in the degradation of prostaglandins is present in the hair follicle dermal papillae, inactivates prostaglandins, especially, PGF2a and PGE2, to cause scalp damage and alopecia (Michelet J F et. al., 2008, Exp. Dermatol, 17(10), 821-8). Thus, the hydroxyprostaglandin dehydrogenase inhibitors described herein that have a suppressive or inhibitory activity against 15-PGDH can improve scalp damage, prevent alopecia and promote hair growth and be used in a pharmaceutical composition for the prevention of alopecia and the promotion of hair growth.

In another aspect, provided herein is a method of preventing and/or treating skin inflammation and/or damage, comprising administering one or more of the compositions described herein to a subject in need thereof.

In another aspect, provided herein is a method of preventing and/or treating vascular insufficiency, comprising administering one or more of the compositions described herein to a subject in need thereof. Prostaglandins including prostaglandin homologues produced in the body have been known to maintain the proper action of the blood vessel wall, especially to contribute to vasodilation for blood flow, preventing platelet aggregation and modulating the proliferation of smooth muscle that surrounds blood vessel walls (Yan. Cheng et. al., 2006, J. Clin., Invest). In addition, the inhibition of prostaglandins production or the loss of their activity causes the degeneration of the endothelium in the blood vessel walls, platelet aggregation and the dysfunction of cellular mechanism in the smooth muscle. Among others, the production of prostaglandins in blood vessels was shown to be decreased in hypertension patients, including pulmonary artery hypertension. the 15-PGDH inhibitors described herein can be used in a pharmaceutical composition for the prevention or the treatment of cardiovascular disease and/or diseases of vascular insufficiency, such as Raynaud's disease, Buerger's disease, diabetic neuropathy, and pulmonary artery hypertension.

In another aspect, provided herein is a method of preventing, treating, minimizing and/or reversing congestive heart failure, cardiomyopathy, comprising administering one or more of the compositions described herein to a subject in need thereof. In another aspect, provided herein is a method of reducing cardiac ejection fraction, comprising administering one or more of the compositions described herein to a subject in need thereof. It has been shown that administration of a 15-PGDH inhibitor can be used to treat, prevent, minimize, and/or reverse congestive heart failure, cardiomyopathy, and/or reduction of cardiac ejection fraction (Markowitz et. al., WO2018/187810). As such, the hydroxyprostaglandin dehydrogenase inhibitors described herein can be administered to a subject in need to treat, prevent, minimize and/or reverse congestive heart failure, cardiomyopathy, and/or reduction of cardiac ejection fraction.

In another aspect, provided herein is a method of preventing and/or treating a gastrointestinal disease, comprising administering one or more of the compositions described herein to a subject in need thereof. Prostaglandins are essential for maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). The inhibitors of hydroxyprostaglandin dehydrogenase described herein show a suppressive or inhibitory activity against 15-PGDH, which degrades prostaglandins that protect gastric mucus membranes. As such, the hydroxyprostaglandin dehydrogenase inhibitors can be effective for the prevention or the treatment of gastrointestinal diseases, inter alia, gastritis and gastric ulcer. In addition, the hydroxyprostaglandin dehydrogenase inhibitors provided herein may be used to prevent and/or treat other forms of intestinal injury including toxicity from radiation and/or chemotherapy, and chemotherapy-induced mucositis.

Additionally, it has been shown that administration of 15-PGDH inhibitors, alone or in combination with corticosteroids and/or TNF inhibitors can treat intestinal, gastrointestinal, or bowel disorders such as oral ulcers, gum disease, gastritis, colitis, ulcerative colitis, gastric ulcers, inflammatory bowel disease, and Crohn's disease (Markowitz et. al., WO 2018/102552). As such, the hydroxyprostaglandin dehydrogenase inhibitors provided herein can be used to treat and/or prevent treat intestinal, gastrointestinal, or bowel disorders such as oral ulcers, gum disease, gastritis, colitis, ulcerative colitis, gastric ulcers, inflammatory bowel disease, and Crohn's disease.

In another aspect, provided herein is a method of preventing and/or treating renal dysfunction, comprising administering one or more of the compositions described herein to a subject in need thereof. In the kidney, prostaglandins modulate renal blood flow and may serve to regulate urine formation by both renovascular and tubular effects. In clinical studies, inhibitors of prostaglandin have been used to improve creatinine clearance in patients with chronic renal disease, to prevent graft rejection and cyclosporine toxicity in renal transplant patients, to reduce the urinary albumin excretion rate and N-acetyl-beta-D-glucosaminidase levels in patients with diabetic nephropathy (Porter, Am., 1989, J. Cardiol., 64: 22E-26E). Furthermore, it has been reported that prostaglandins serve as vasodilators in the kidney, and, thus, the inhibition of prostaglandin production in the kidney results in renal dysfunction (Hao. C M, 2008, Annu Rev Physiol, 70, 357.about.77). The hydroxyprostaglandin dehydrogenase inhibitors described herein have a suppressive or inhibitory activity against 15-PGDH that degrades prostaglandins and can be used for the prevention and/or treatment of renal diseases that are associated with renal dysfunction.

In another aspect, provided herein is a method of stimulation bone resorption and bone formation, comprising administering one or more of the compositions described herein to a subject in need thereof. Prostaglandins have been shown to stimulate bone resorption and bone formation to increase the volume and the strength of the bone (H. Kawaguchi et. al., Clinical Orthop. Rel. Res., 313, 1995; J. Keller et al., Eur. Jr. Exp. Musculoskeletal Res., 1, 1992, 8692). Furthermore, inhibition of 15-PGDH increases callus size and mineralization after bone fracture (Collier et. al., ORS 2017 Annual Meeting Paper No. 0190). Considering that 15-PGDH inhibits the activities of prostaglandins as mentioned in the above, the inhibition of 15-PGDH activity may lead to the promotion of bone resorption and bone formation that are inhibited by 15-PGDH. Thus, the inhibitors of hydroxyprostaglandin dehydrogenase described herein can be effective for the promotion of bone resorption and bone formation by inhibiting 15-PGDH activity. The hydroxyprostaglandin dehydrogenase inhibitors provided herein can also be used to increase bone density, treat osteoporosis, promote healing of fractures, promote healing after bone surgery or joint replacement, and/or to promote healing of bone to bone implants, bone to artificial implants, dental implants, and bone grafts.

In another aspect, provided herein is a method of stimulating tissue regeneration by stimulating, comprising administering one or more of the compositions described herein to a subject in need thereof. Prostaglandin PGE2 supports expansion of several types of tissue stem cells. Inhibition of 15-hydroxyprostaglandin dehydrogenase (15-PGDH), a prostaglandin-degrading enzyme, potentiates tissue regeneration in multiple organs. Studies show that inhibition of 15-PGDH increases prostaglandin PGE2 levels in bone marrow and other tissues; accelerates hematopoietic recovery following a bone marrow transplant; promotes tissue regeneration of colon and liver injury (Zhang, Y. et. al. Science 2015, 348 (6240)). The hydroxyprostaglandin dehydrogenase inhibitors provided herein can be used for tissue regeneration by supporting the expansion of tissue stem cells.

In another aspect, provided herein is a method of modulating cervical ripening, comprising administering one or more of the compositions described herein to a subject in need thereof. Prostaglandin E2 (PGE2) is a known cervical ripening agent that mediates EP2-receptor-signaling pathways in human cervical stromal cells; targets its own synthesis by increasing COX-2 and PTGES expression; and decreases its metabolism by loss of its degradative enzyme 15-PGDH (Word et. A1, WO2019010482) Downregulation of 15-PGDH was also found to be crucial for PGE2-induced cervical ripening and preterm birth. Modulation of 15-PDGH activity can be used to modulate cervical ripening; and induce or prevent preterm labor. The hydroxyprostaglandin dehydrogenase inhibitors provided herein can be used to induce cervical ripening and labor, alone or in combination with another labor inducing agent.

In another aspect, provided herein is a method of promoting neuroprotection and/or stimulating neuronal regeneration, comprising administering one or more of the compositions described herein to a subject in need thereof. Prostaglandins, via their specific G protein coupled receptors, have a variety of physiological functions in the central nervous system. The major prostaglandin, prostaglandin E2 (PGE2) can activate receptor types EP1, 2, 3, and 4. Activation of EP2 and EP4 receptors can regulate adenylate cyclase and the generation of 3, 5′-cyclic adenosine monophosphate (cAMP), whereas the activation of EP1 and EP3 receptors can regulate Ca2+ signaling. Studies show that the EP1 and EP2 receptors are expressed in neurons and microglia as well as neurons of the cerebral cortex, striatum, and hippocampus. In addition, activation of the EP2 receptor by PGE2 is involved in long-term synaptic plasticity and cognitive function (Chemtob et al. Semin Perinatol. 1994 February; 18(1):23-9; Yang et al., J Neurochem. 2009 January; 108(1):295-304). Studies also show that following activation, different PGE2 receptors can contribute or protect against N-methyl-D-aspartate (NMDA) neurotoxicity and ischemic stroke (Ahmad et al., Exp Transl Stroke Med. 2010 Jul. 8; 2(1):12). Other studies show that activation of the EP2 receptors protected neurons from amyloid β-peptide neurotoxicity in vitro (Echeverria et al., Eur J Neurosci. 2005 November; 22(9):2199-206). Several studies suggest that the mechanism by which PGE2 affords neuroprotection is through EP2 or EP4 receptors, as they both increases cAMP, followed by a protein kinase A (PKA)-dependent pathway (Echeverria et al. Eur J Neurosci. 2005 November; 22(9):2199-206; McCullough et al., J Neurosci. 2004 Jan. 7; 24(1):257-68). Stimulation of these receptors with PGE2 by administration of a compound that inhibits, reduces, and/or antagonizes 15-PGDH activity, such as the hydroxyprostaglandin dehydrogenase inhibitors that can inhibit 15-PGDH described herein, can promote neuroprotection in a subject from axonal degeneration, neuronal cell death, and/or glia cell damage after injury, augment neuronal signaling underlying learning and memory, stimulate neuronal regeneration after injury, and/or treat diseases, disorders, and/or conditions of the nervous system.

In another aspect, provided herein is a method of treating and/or preventing a neurological disorder, a neuropsychiatric disorder, a neural injury, a neural toxicity disorder, a neuropathic pain, or a neural degenerative disorder, comprising administering one or more of the compositions described herein to a subject in need thereof. In some embodiments, the disease, disorder, and/or condition of the nervous system, which can be treated with hydroxyprostaglandin dehydrogenase inhibitors provided herein, can include at least one of a neurological disorder, a neuropsychiatric disorder, a neural injury, a neural toxicity disorder, a neuropathic pain, or a neural degenerative disorder. For example, the neurological disorder can include at least one of traumatic or toxic injuries to peripheral or cranial nerves, spinal cord or brain, such as traumatic brain injury, stroke, cerebral aneurism, and spinal cord injury. The neurological disorder can also include at least one of Alzheimer's disease, dementias related to Alzheimer's disease, Parkinson's, Lewy diffuse body diseases, senile dementia, Huntington's disease, Gilles de Ia Tourette's syndrome, multiple sclerosis, amyotrophic lateral sclerosis, hereditary motor and sensory neuropathy, diabetic neuropathy, progressive supranuclear palsy, epilepsy, or Jakob-Creutzfieldt disease.

In some embodiments, the neural injury can be caused by or associated with at least one of epilepsy, cerebrovascular diseases, autoimmune diseases, sleep disorders, autonomic disorders, urinary bladder disorders, abnormal metabolic states, disorders of the muscular system, infectious and parasitic diseases, neoplasms, endocrine diseases, nutritional and metabolic diseases, immunological diseases, diseases of the blood and blood-forming organs, mental disorders, diseases of the nervous system, diseases of the sense organs, diseases of the circulatory system, diseases of the respiratory system, diseases of the digestive system, diseases of the genitourinary system, diseases of the skin and subcutaneous tissue, diseases of the musculoskeletal system and connective tissue, congenital anomalies, or conditions originating in the perinatal period.

In certain embodiments, the hydroxyprostaglandin dehydrogenase inhibitors can be administered to a subject or neurons of the subject to promote the survival, growth, development and/or function of the neurons, particularly, the central nervous system (CNS), brain, cerebral, and hippocampal neurons. In certain embodiments, the hydroxyprostaglandin dehydrogenase inhibitors can be used stimulate hippocampal neurogenesis, for the treatment of neuropsychiatric and neurodegenerative diseases, including (but not limited to) schizophrenia, major depression, bipolar disorder, normal aging, epilepsy, traumatic brain injury, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, and abuse of neuro-active drugs, such as alcohol, opiates, methamphetamine, phencyclidine, and cocaine.

In another aspect, provided herein is a method of treating and/or preventing fibrotic or adhesion disease, disorder or condition, comprising administering one or more of the compositions described herein to a subject in need thereof. It has been shown that inhibitors of short-chain dehydrogenase activity, such as 15-PGDH inhibitors, can be administered to a subject in need thereof to decrease fibrotic symptoms, such as collagen deposition, collagen accumulation, collagen fiber formation, inflammatory cytokine expression, and inflammatory cell infiltration, and treat and/or prevent various fibrotic diseases, disorders, and conditions characterized, in whole or in part, by the excess production of fibrous material, including excess production of fibrotic material within the extracellular matrix, or the replacement of normal tissue elements by abnormal, non-functional, and/or excessive accumulation of matrix-associated components (Markowitz et. al., WO2016/144958).

Fibrotic diseases, disorders and conditions characterized, in whole or in part, by excess production of fibrotic material can include systemic sclerosis, multifocal fibrosclerosis, nephrogenic systemic fibrosis, scleroderma (including morphea, generalized morphea, or linear scleroderma), sclerodermatous graft- vs-host-disease, kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g. pulmonary fibrosis, glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fasciitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, myelodysplastic syndrome, myeloproliferative syndrome, collagenous colitis, acute fibrosis, organ specific fibrosis, and the like. The hydroxyprostaglandin dehydrogenase inhibitors provided herein can be used to treat or prevent a fibrotic disease, disorder or condition.

The hydroxyprostaglandin dehydrogenase inhibitors provided herein can be used to treat or prevent kidney fibrosis, including kidney fibrosis resulting from dialysis following kidney failure, catheter placement, a nephropathy, glomerulosclerosis, glomerulonephritis, chronic renal insufficiency, acute kidney injury, end stage renal disease or renal failure, or combinations thereof.

The hydroxyprostaglandin dehydrogenase inhibitors provided herein can be used to treat or prevent liver fibrosis, including liver fibrosis resulting from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha-1-antitrypsin deficiency, primary biliary cirrhosis, drug reaction and exposure to toxins, or combinations thereof.

The hydroxyprostaglandin dehydrogenase inhibitors provided herein can be used to treat or prevent heart fibrosis such as cardiac fibrosis, endomyocardial fibrosis, idiopathic pulmonary fibrosis, and kidney fibrosis.

The hydroxyprostaglandin dehydrogenase inhibitors provided herein can be used to treat or prevent systemic sclerosis.

The hydroxyprostaglandin dehydrogenase inhibitors provided herein can be used to treat or prevent fibrotic diseases, disorders or conditions caused by post-surgical adhesion formation.

The hydroxyprostaglandin dehydrogenase inhibitors provided herein can be used to reduce in intensity, severity, or frequency, and/or delay onset of one or more symptoms or features of a fibrotic disease, disorder or condition, or other related diseases, disorders or conditions.

The hydroxyprostaglandin dehydrogenase inhibitors provided herein can be used to decrease or reduce collagen secretion, or collagen deposition, or collagen fiber accumulation, in a tissue or organ, such as the lung, the liver, the intestines, the colon, the skin or the heart, or a combination thereof.

Studies have shown that 15-PGDH inhibition ameliorates inflammatory pathology and fibrosis in pulmonary fibrosis (Smith et. al., bioRxiv 2019.12.16.878215; Barnthaler et. al., J. Allergy Clin. Immunol. 2019, 145 (3), 818-833). In some embodiments, the hydroxyprostaglandin dehydrogenase inhibitors described herein can be used to treat or prevent lung fibrosis, including pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

In another aspect, provided herein is a method of reducing and/or preventing scar formation, comprising administering one or more of the compositions described herein to a subject in need thereof. The hydroxyprostaglandin dehydrogenase inhibitors provided herein can used for reducing or preventing scar formation in a subject. The hydroxyprostaglandin dehydrogenase inhibitors provided herein can be used to reduce or prevent scar formation on skin or scleroderma.

In another aspect, provided herein is a method of treating and/or preventing muscle disorder, muscle injury and/or muscle atrophy, comprising administering one or more of the compositions described herein to a subject in need thereof. Studies have shown that inhibition of PGE2 degrading enzymes such as 15-PGDH, enable muscle regeneration and muscle repair after injury (Ho et al., PNAS 2017; Dong et al., Stem cell research and therapy 2020). The inhibitors of hydroxyprostaglandin dehydrogenase provided herein can be used to treat muscle disorder, muscle injury and/or muscle atrophy in a subject. In some cases, said subject suffering from a muscle disorder, muscle injury and/or muscle atrophy may have Duchenne muscular dystrophy (DMD), Becker muscular dystrophy, Fukuyama congenital muscular dystrophy (FCMD), limb girdle muscular dystrophy, congenital muscular dystrophy, facioscapulohumeral muscular dystrophy (FHMD), amyotrophic lateral sclerosis (ALS), distal muscular dystrophy (DD), an inherited myopathy, myotonic muscular dystrophy (MDD), oculopharyngeal muscular dystrophy, distal muscular dystrophy, Emery-Dreifuss muscular dystrophy, myotonia congenita, mitochondrial myopathy (DD), myotubular myopathy (MM), myasthenia gravis (MG), periodic paralysis, polymyositis, rhabdomyolysis, dermatomyositis, cancer cachexia, AIDS cachexia, stress induced urinary incontinence, urethral sphincter deficiency, sarcopenia, or a combination thereof.

In some embodiments, the inhibitors of hydroxyprostaglandin dehydrogenase provided herein can be used to treat sarcopenia. In another embodiment, the inhibitors of hydroxyprostaglandin dehydrogenase provided herein can be used to treat diaphragmatic atrophy or limb muscle atrophy due to the use of a mechanical ventilator. In some embodiments, the inhibitors of hydroxyprostaglandin dehydrogenase provided herein can be used to treat genetic disorders or neuromuscular disorders such as Spinal Muscular Atrophy (SMA). In some embodiments, the inhibitors of hydroxyprostaglandin dehydrogenase provided herein can be used to treat ptosis, rotator cuff muscle atrophy, immobilization related muscle atrophy, surgical procedure related muscle atrophy, sarcopenia, or a combination thereof.

### Pharmaceutical Compositions

The inhibitors of hydroxyprostaglandin dehydrogenase can be formulated into pharmaceutical compositions to treat diseases and disorders described herein. In some embodiments, a pharmaceutical composition may comprise a therapeutically effective amount of one or more inhibitors of hydroxyprostaglandin dehydrogenase provided herein.

The pharmaceutical composition described herein may be administered in such oral dosage forms as tablets, capsules (each of which includes sustained release or timed release formulations), pills, powders, micronized compositions, granules, elixirs, tinctures, suspensions, ointments, vapors, liposomal particles, nanoparticles, syrups and emulsions. In some embodiments, the pharmaceutical composition may also be administered in intravenous (bolus or infusion), subcutaneous injection, suppository, intraperitoneal, topical (e.g., dermal epidermal, transdermal), ophthalmically such as ocular eyedrop, intranasally, subcutaneous, inhalation, intramuscular or transdermal (e.g., patch) form, all using forms well known to those of ordinary skill in the pharmaceutical arts.

In some embodiments, a compound provided herein can be administered as part of a therapeutic regimen that comprises administering one or more second agents (e.g. 1, 2, 3, 4, 5, or more second agents), either simultaneously or sequentially with the compound provided herein. When administered sequentially, the compound provided herein may be administered before or after the one or more second agents. When administered simultaneously, the compound provided herein and the one or more second agents may be administered by the same route (e.g. injections to the same location; tablets taken orally at the same time), by a different route (e.g. a tablet taken orally while receiving an intravenous infusion), or as part of the same combination (e.g. a solution comprising a compound provided herein and one or more second agents).

A combination treatment according to the disclosure may be effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. The exact dosage will depend upon the agent selected, the route of administration, the form in which the compound is administered, the subject to be treated, the body weight of the subject to be treated, and the preference and experience of the attending physician.

### EXAMPLES

The following examples are offered to illustrate, but not to limit the claimed invention. The following examples further illustrate the invention but, of course, should not be construed as in any way limiting its scope.

### Synthesis and Characterization of Compounds

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Exemplary synthesis schemes for the inhibitors with phenyl core as described herein include:

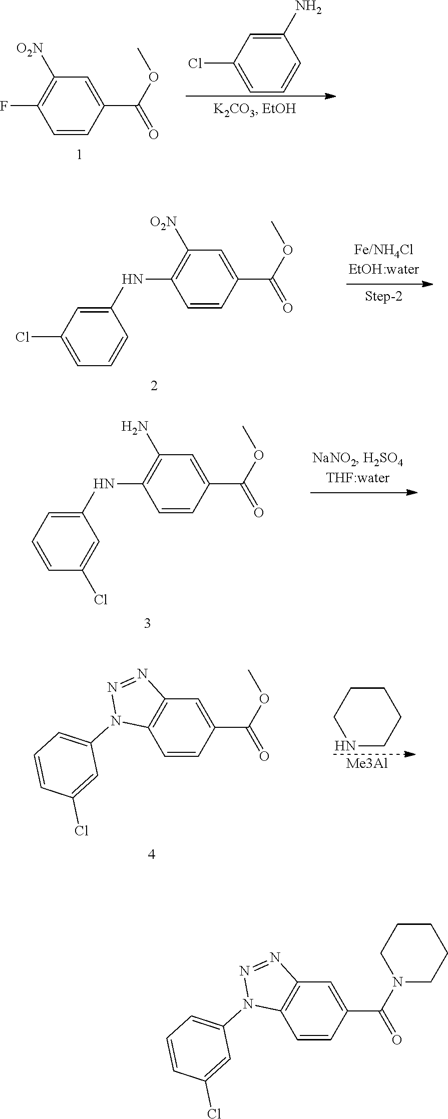

In some cases, synthesis schemes may be entire synthesis schemes for producing the inhibitors provided herein. In other cases, synthesis schemes may be partial schemes for producing inhibitors provided herein.

Described herein are exemplary synthesis schemes that can be used to synthesize the inhibitors described herein. The following abbreviations are used:

| Abbreviation | Description |
|---|---|
| AIBN | azobisisobutyronitrile |
| DCM | dichloromethane |
| DIAD | diisopropyl azodicarboxylate |
| DIPEA | N,N′-diisopropylethylamine |
| DMAP | 4-dimethylaminopyridine |
| DMF | dimethylformamide |
| EDCI | 1-ethyl-3-(3-dimethylaminopropyl)carbodiimide |
| HATU | 1-[Bis(dimethylamino)methylene]-1H-1,2,3- |
| | triazolo[4,5-b]pyridinium 3-oxide hexafluorophosphate |
| HOBt | hydroxybenzotriazole |
| m-CPBA | Meta-chloroperoxybenzoic acid |
| NBS | N-bromosuccinimide |
| NCS | N-chlorosuccinimide |
| NIS | N-iodosuccinimide |
| p-TSA | para-toluenesulfonic acid |
| TEA | triethylamine |
| TFA | trifluoroacetic acid |
| THF | tetrahydrofuran |
| TPP | triphenylphosphine |
| mmol | Milli molar |
| vol | Volume |
| g | Gram |
| kg | Kilogram |
| L | Liter |
| mL | Milliliter |
| ° C. | Degree Celsius |
| TLC | Thin Layer Chromatography |
| HPLC | High-performance liquid chromatography |
| LCMS | Liquid chromatography-mass spectrometry |
| min | Minutes |
| h | Hour |
| eq | Equivalents |
| RT | Room temperature |
| Rf | Retention factor |
| RP | Reversed phase |
| NMR | Nuclear magnetic resonance |
| Ppm | Parts per million |

Example 1. Synthesis of 4-(6-(4,4-difluoropiperidine-1-carbonyl)-3H-[1,2,3]triazolo[4,5-b]pyridin-3-yl)benzoic acid (C-1) and 4-(6-(4,4-difluoropiperidine-1-carbonyl)-2-methyl-3H-imidazo[4,5-b]pyridin-3-yl)benzoic acid (C-3)

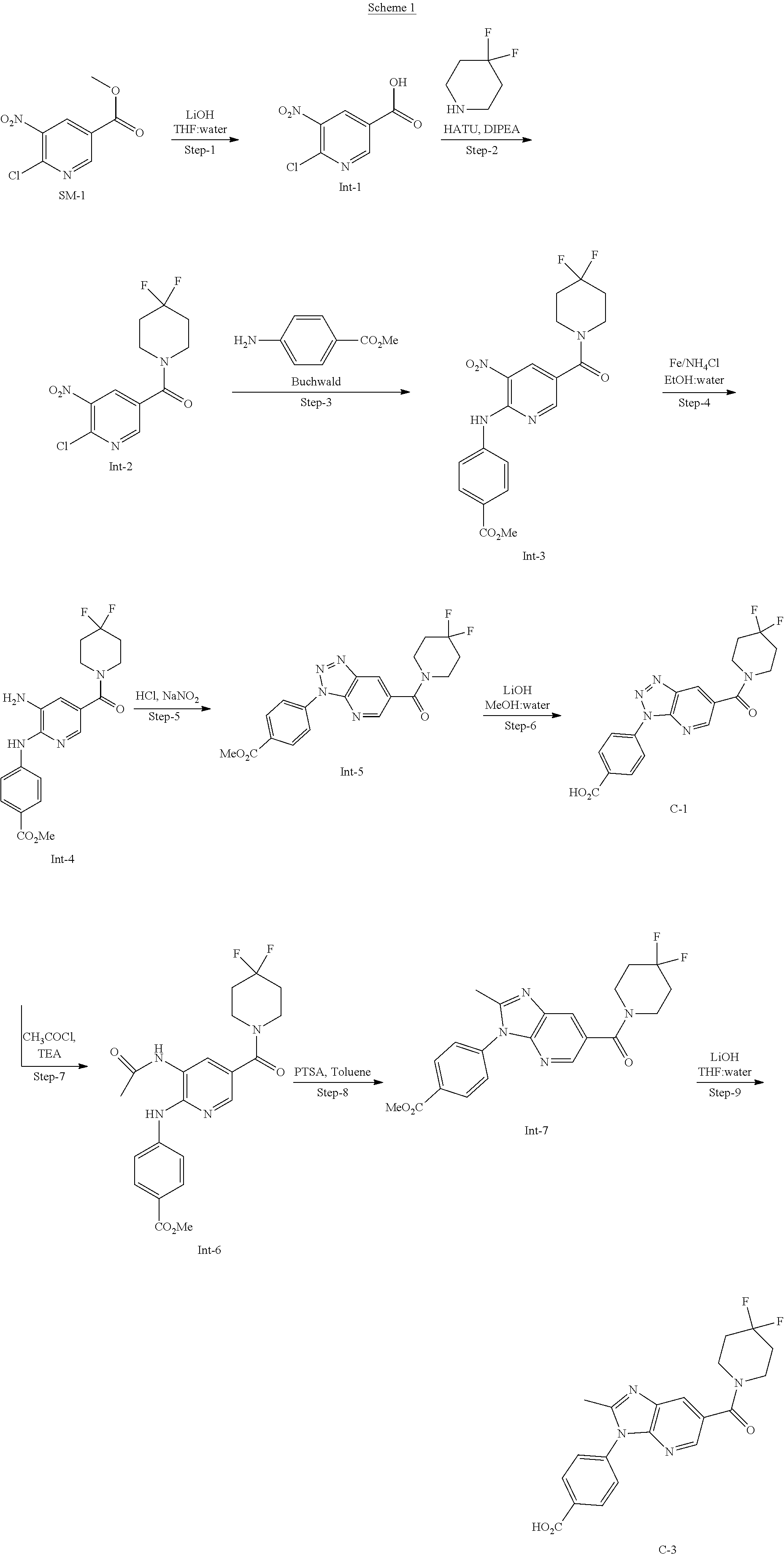

Step-1: Synthesis of 6-chloro-5-nitronicotinic acid, general procedure for ester hydrolysis with LiOH: To a stirred solution of methyl 6-chloro-5-nitronicotinate, (SM-1) (5 g, 23.09 mmol, 1.0 eq) in THF:water (1:1, 20 mL) at 0° C., LiOH·H₂O (2.8 g, 69.27 mmol, 3 eq) was added at 0° C. The resultant reaction mixture was stirred at RT for 4 h. The reaction was monitored by crude LCMS/TLC; after completion of the reaction, volatiles were evaporated, neutralized with satd. aqueous citric acid up to PH=7. Filtered solids were washed with Et₂O (50 mL) and dried in vacuo to obtain 6-chloro-5-nitronicotinic acid, Int-1 (3 g) as yellow solid. The crude was used in the next step without further purification. TLC: 80% EtOAc/Heptane (Rf: 0.15). MS: m/z=203.5 [M+H]⁺.

Step-2: Synthesis of (6-chloro-5-nitropyridin-3-yl)(4,4-difluoropiperidin-1-yl) methanone (Int-2) (General procedure for acid-amine coupling using HATU): To the stirred solution of 6-chloro-5-nitronicotinic acid, Int-1 (3 g, 14.81 mmol, 1.0 eq.) in DMF (20 mL) at 0° C., HATU (6.75 g, 17.77 mmol, 1.2 eq), 4,4-difluoropiperidine hydrochloride (2.80 g, 17.17 mmol, 1.2 eq) were added. To this stirred solution N, N′-diisopropylethylamine (7.76 mL, 44.43 mmol, 3 eq) was added at 0° C. and then continued for stirring at RT for 16 h. The progress of the reaction was monitored with TLC and LCMS. After consumption of starting material, the mixture was diluted with ice cold water (10 mL) and extracted with EtOAc (3×30 mL). The combined extracts were washed with water and brine, dried over sodium sulphate, filtered and concentrated under reduced pressure. The crude product was purified by combi-flash column chromatography using 70% EtOAc:heptane to afford (6-chloro-5-nitropyridin-3-yl)(4,4-difluoropiperidin-1-yl)methanone, Int-2 (2 g, 46.70%) as off white solid. TLC: 40% EtOAc/Heptane (Rf: 0.40) MS: m/z=306.6 [M+H]⁺.

Step-3: Synthesis of methyl 4-((5-(4,4-difluoropiperidine-1-carbonyl)-3-nitropyridin-2-yl)amino) benzoate (Int-3) (General procedure for Buchwald coupling): To a stirring solution of (6-chloro-5-nitropyridin-3-yl)(4,4-difluoropiperidin-1-yl)methanone (Int-2) (1 g, 3.27 mmol, 1.0 eq) in dioxane (30 mL) in a sealed tube under inert atmosphere, Cs₂CO₃ (3.18 g, 9.83 mmol, 3 eq), methyl 4-aminobenzoate (SM-2) (593 mg, 3.93 mmol, 1.2 eq) were added at RT. Argon gas was purged for 15 min then Xantphos (378 mg, 0.654 mmol, 0.2 eq) and Pd₂(dba)₃ (300 mg, 0.327 mmol, 0.1 eq), were added under argon atmosphere, then resultant reaction mixture was heated in the microwave at 150° C. for 20 min. The reaction was monitored by crude LCMS/TLC; after consumption of starting material, the mixture was quenched with saturated NH₄Cl (10 mL), filtered through a celite bed and washed with EtOAc (2×50 mL). The combined EtOAc extracts were washed with brine (100 mL); dried over sodium sulphate, filtered and concentrated in vacuo to obtain the crude. The crude was purified by combi-flash column purification using 60% EtOAc:heptane to afford methyl 4-((5-(4,4-difluoropiperidine-1-carbonyl)-3-nitropyridin-2-yl)amino) benzoate, Int-3 (555 mg) as a brownish semi-solid. The crude was used in the next step without further purification. TLC: 80% EA: Hex (R_{f}: 0.5). MS: m/z=421.2 [M+H]⁺.

Step-4: Synthesis of methyl 4-((3-amino-5-(4,4-difluoropiperidine-1-carbonyl)pyridin-2-yl)amino)benzoate (Int-4), general procedure for reduction of aryl nitro compounds: To a stirred solution of compound methyl 4-((3-amino-5-(4,4-difluoropiperidine-1-carbonyl)pyridin-2-yl)amino)benzoate Int-3 (500 mg, 1.19 mmol, 1.0 eq.) in ethanol (5 mL) was added Fe (5.0 eq) followed by ammonium chloride (10 eq.) in water (5 mL) and the reaction mixture was heated at 80° C. for 3 h. After completion of the reaction (monitored by TLC), the reaction mixture was filtered and filtrate was concentrated under reduced pressure. The crude residue was suspended in water and extracted with EtOAc. The combined organic layers were dried over anhydrous Na₂SO₄, filtered, and concentrated under reduced pressure to afford methyl 4-((3-amino-5-(4,4-difluoropiperidine-1-carbonyl)pyridin-2-yl) amino)benzoate (Int-4) (118 mg, 25.43%) as pale brown solid which was used for next step without purification. TLC: 60% EtOAc:heptane (R_{f}: 0.3); MS: m/z=391.2 [M+H]⁺.

Step-5: Synthesis of methyl 4-(6-(4,4-difluoropiperidine-1-carbonyl)-3H-[1,2,3]triazolo[4,5-b]pyridin-3-yl) benzoate (Int-5): To a solution of methyl 4-((3-amino-5-(4,4-difluoropiperidine-1-carbonyl)pyridin-2-yl) amino) benzoate (Int-4) (110 mg, 0.282 mmol, 1 eq) dissolved in 1, 4-dioxane (5 mL) was added NaNO₂ (38.91 mg, 0.564 mmol, 2 eq) at 0° C. followed by catalytic amount of HCl (conc). The mixture was stirred at 30° C. for 12 h. The reaction was monitored by TLC; after consumption of starting material, it was quenched with ice-water (10 mL), extracted with EtOAc (3×10 mL), combined extracts were washed with brine (10 mL), dried over sodium sulphate, filtered and concentrated in vacuo to obtain the crude. The crude product was purified by combi-flash column chromatography using 80% EtOAc:heptane to afford methyl 4-(6-(4,4-difluoropiperidine-1-carbonyl)-3H-[1,2,3]triazolo[4,5-b]pyridin-3-yl)benzoate, Int-5 (69 mg, 61.06%) as an off white solid. TLC: 80% EtOAc:heptane (R_{f}: 0.2); MS: m/z=402.1 [M+H]⁺.

Step-6: Synthesis of 4-(6-(4,4-difluoropiperidine-1-carbonyl)-3H-[1,2,3]triazolo[4,5-b]pyridine-3-yl) benzoic acid (C-1): methyl 4-(6-(4,4-difluoropiperidine-1-carbonyl)-3H-[1,2,3]triazolo[4,5-b]pyridin-3-yl)benzoate, (Int-5) (55 mg, 0.137 mmol, 1 eq) was converted to 4-(6-(4,4-difluoropiperidine-1-carbonyl)-3H-[1,2,3]triazolo[4,5-b] pyridine-3-yl) benzoic acid (C-1) using general procedure for ester hydrolysis with LiOH to afford 4-(6-(4,4-difluoropiperidine-1-carbonyl)-3H-[1,2,3]triazolo[4,5-b]pyridine-3-yl) benzoic acid (C-1) (24 mg, 45.28%) as an off white solid. TLC: 80% EtOAc:heptane (R_{f}: 0.2); LCMS: 95.63%, MS: m/z=388.1 [M+H]⁺.

Step-7: Synthesis of methyl 4-((3-acetamido-5-(4,4-difluoropiperidine-1-carbonyl)pyridin-2-yl)amino)benzoate (Int-6): To a stirred solution of methyl 4-((3-amino-5-(4,4-difluoropiperidine-1-carbonyl)pyridin-2-yl)amino)benzoate, (Int-4) (165 mg, 0.422 mmol, 1 eq) in anhydrous DCM (5 mL) cooled to 0° C. was added trimethylamine (0.115 mL, 0.84 mmol, 2 eq) followed by acetyl chloride (0.036 mL, 0.50 mmol, 1.2 eq). The mixture was stirred at 30° C. for 6 h. The reaction was monitored by crude LCMS/TLC; after completion the reaction was quenched with ice-water (10 mL) and extracted with DCM (3×10 mL). The combined extracts were washed with brine (10 mL), dried over sodium sulphate, filtered and concentrated in vacuo to obtain the crude. The crude product was purified by combi-flash column chromatography using 80% EtOAc:heptane to afford methyl 4-((3-acetamido-5-(4,4-difluoropiperidine-1-carbonyl)pyridin-2-yl) amino) benzoate (Int-6) (116 mg, 66%) as an brown sticky oil. TLC: 100% EtOAc (R_{f}: 0.4)

Step-8: Synthesis of methyl 4-(6-(4,4-difluoropiperidine-1-carbonyl)-2-methyl-3H-imidazo[4,5-b]pyridin-3-yl)benzoate (Int-7), general procedure for cyclization to make imidazole: To a stirred solution methyl 4-((3-acetamido-5-(4,4-difluoropiperidine-1-carbonyl)pyridin-2-yl)amino)benzoate, (Int-6) (116 mg, 0.26 mmol, 1 eq) in toluene (5 mL) was added PTSA (20 mg, 0.133 mmol, 0.5 eq) followed by 4 A° molecular sieves. The mixture was stirred at reflux for 12 h. The reaction was monitored by TLC and after completion, the solvent was removed in vacuo. The crude product was triturated with n-pentane to afford methyl 4-(6-(4,4-difluoropiperidine-1-carbonyl)-2-methyl-3H-imidazo [4,5-b]pyridin-3-yl)benzoate, (44 mg) as an brown sticky oil. TLC: 20% Acetone:Toluene (R_{f}: 0.4); MS: m/z=415.1 [M+H]⁺.

Step-9: Synthesis of 4-(6-(4,4-difluoropiperidine-1-carbonyl)-2-methyl-3H-imidazo[4,5-b]pyridin-3-yl)benzoic acid (C-3): Int-7 (40 mg, 0.096 mmol, 1 eq)) was hydrolyzed using the general procedure for ester hydrolysis with LiOH to afford 4-(6-(4,4-difluoropiperidine-1-carbonyl)-2-methyl-3H-imidazo[4,5-b]pyridin-3-yl)benzoic acid (C-3) (31.3 mg, 81%) MS: m/z=401.1 [M+H]⁺.

Example 2. Synthesis of (1-(6-(1H-1,2,4-triazol-5-yl)pyridin-3-yl)-1H-benzo[d]imidazol-5-yl)(4,4-difluoropiperidin-1-yl)methanone (C-15), (1-(4-(1H-1,2,4-triazol-5-yl)phenyl)-1H-benzo[d]imidazol-5-yl)(4,4-difluoropiperidin-1-yl)methanone (C-16), and 4-(5-(4,4-difluoropiperidine-1-carbonyl)-1H-benzo[d]imidazol-1-yl) benzoic acid (C-17)

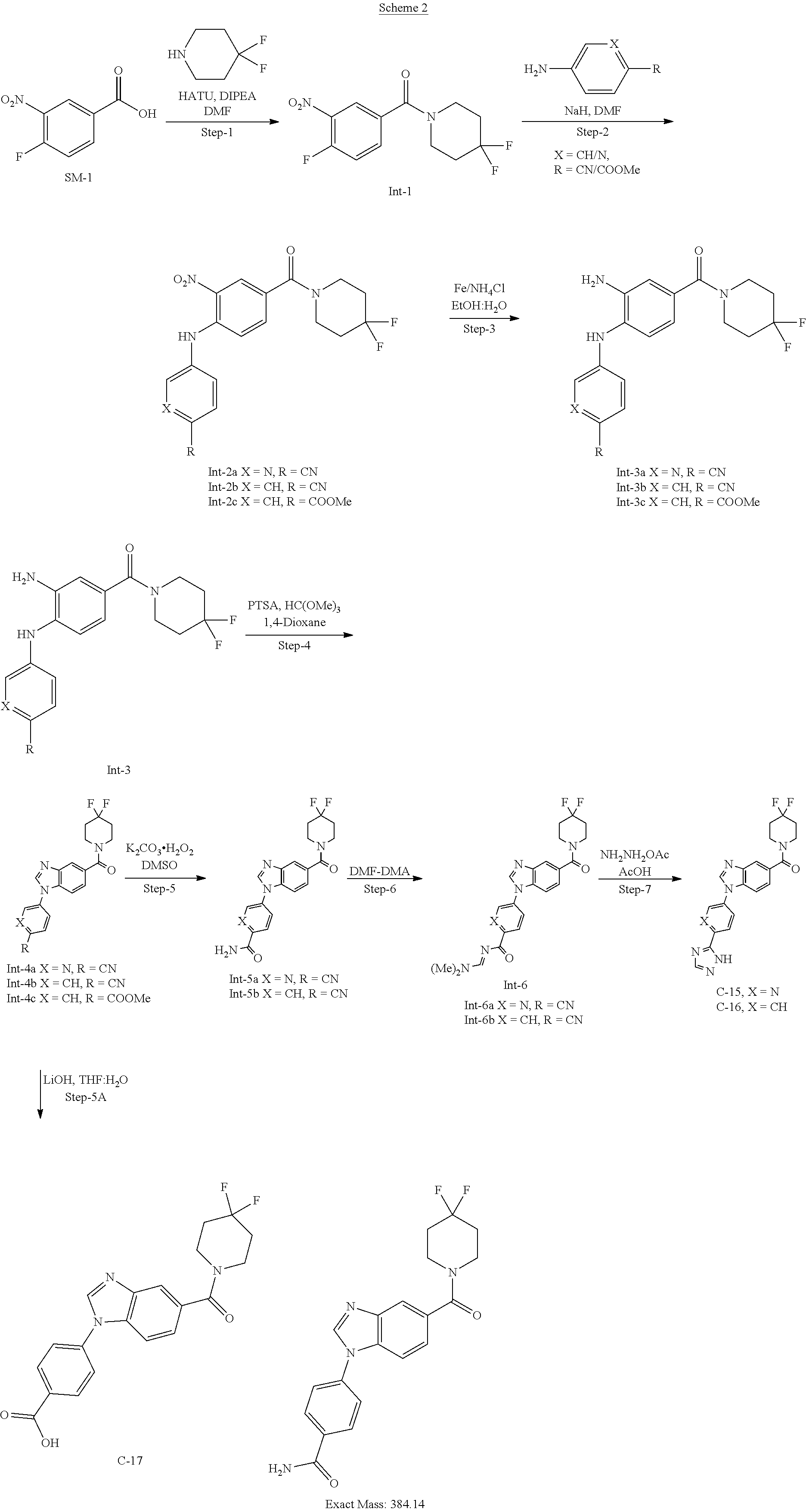

Step-1: Int-1: Previously described in the synthesis of C-9.

Step-2: Synthesis of Int-2a, Int-2b and Int-2c: (4,4-difluoropiperidin-1-yl)(4-fluoro-3-nitrophenyl) methanone, Int-1 using the general procedure for SNAr reaction described earlier using appropriate amines to afford Int-2a, Int2b and Int2c.

Int-2a (for C-15): (74.50% yield, m/z=388.1 [M+H]⁺).

Int-2b (for C-16): (57% yield, m/z=387.1 [M+H]⁺).

Int-2c (for C-17): (56% yield, m/z=420.1 [M+H]⁺).

Step-3: Synthesis of Int-3a, Int-3b and Int-3c: Int-2a, Int-2b, and Int-2c was subjected to the general procedure for aryl nitro reduction with Fe. The crude was purified by combiflash column chromatography using 60% EtOAc/heptane to obtain Int-3a, Int-3b and Int-3c.

Int-3a (for C-15): (38% yield, m/z=358.1 [M+H]⁺).

Int-3b (for C-16): (56% yield, m/z=357.1 [M+H]⁺).

Int-3c (for C-17): (98.10% yield, m/z=390.2 [M+H]⁺).

Step-4: Synthesis of Int-4a, Int-4b and Int-4c: Int-3a, Int-3b and Int-3c, was converted to Int-4a, Int-4b and Int-4c using the general procedure for imidazole cyclisation with PTSA and trimethyl orthoformate to afford Int-4a, Int-4b and Int-4c

Int-4a (for C-15): (60.80% yield, m/z=368.1 [M+H]⁺).

Int-4b (for C-16): (56% yield, m/z=367.13 [M+H]⁺).

Int-4c (for C-17): (54.7% yield, m/z=400.2 [M+H]⁺).

Step-5: Synthesis of Int-5a and Int-5b: Int-4a and Int-4b was converted to Int-5a and Int-5b using the general procedure for the oxidation of nitriles to afford crude Int-5a and Int-5b. The crude obtained was triturated with n-pentane filtered and dried in vacuo.

Int-5a (for C-15): (100% crude yield, m/z=386.2 [M+H]⁺)

Int-5b (for C-16): (100% crude yield, m/z=385.14 [M+H]⁺)

Step-5A: Synthesis of 4-(5-(4,4-difluoropiperidine-1-carbonyl)-1H-benzo[d]imidazol-1-yl) benzoic acid, C-17: methyl 4-(5-(4,4-difluoropiperidine-1-carbonyl)-1H-benzo[d]imidazol-1-yl)benzoate, Int-4c (280 mg, 0.701 mmol, 1 eq) using the general procedure for ester hydrolysis with LiOH to afford 4-(5-(4,4-difluoropiperidine-1-carbonyl)-1H-benzo[d]imidazol-1-yl) benzoic acid, C-17 (52 mg, 19.28%) MS: m/z=386.2 [M+H]⁺.

Step-6: Synthesis of (E)-5-(5-(4,4-difluoropiperidine-1-carbonyl)-1H-benzo[d]imidazol-1-yl)-N-((dimethylamino)methylene)picolinamide, Int-6a and (E)-4-(5-(4,4-difluoropiperidine-1-carbonyl)-1H-benzo[d]imidazol-1-yl)-N-((dimethylamino) methylene) benzamide, Int-6b: Int-5a and Int-5b was converted to (E)-5-(5-(4,4-difluoropiperidine-1-carbonyl)-1H-benzo[d]imidazol-1-yl)-N-((dimethylamino)methylene)picolinamide, Int-6a and (E)-4-(5-(4,4-difluoropiperidine-1-carbonyl)-1H-benzo[d]imidazol-1-yl)-N-((dimethylamino) methylene) benzamide, Int-6b using general procedure for enamine formation to afford crude Int-6a and Int-6b. The crude obtained was triturated with n-pentane filtered and dried in vacuo to afford.

Int-6a (for C-15): (100% crude yield, m/z=441.2 [M+H]⁺).

Int-6b (for C-16): (100% crude yield, m/z=440.1 [M+H]⁺).

Step-7: Synthesis of (1-(6-(1H-1,2,4-triazol-5-yl)pyridin-3-yl)-1H-benzo[d]imidazol-5-yl) (4,4-difluoropiperidin-1-yl)methanone, MH-DH-650 and (1-(4-(1H-1,2,4-triazol-5-yl) phenyl)-1H-benzo[d]imidazol-5-yl) (4,4-difluoropiperidin-1-yl)methanone, C-16: Int-6a and Int-6b was converted to (1-(6-(1H-1,2,4-triazol-5-yl)pyridin-3-yl)-1H-benzo[d]imidazol-5-yl) (4,4-difluoropiperidin-1-yl)methanone, C-15 and (1-(4-(1H-1,2,4-triazol-5-yl) phenyl)-1H-benzo[d]imidazol-5-yl) (4,4-difluoropiperidin-1-yl) methanone, C-16 using general procedure for triazole formation using hydrazine acetate to afford (1-(6-(1H-1,2,4-triazol-5-yl)pyridin-3-yl)-1H-benzo[d]imidazol-5-yl)(4,4-difluoropiperidin-1-yl) methanone, C-15 as an off-white solid and (1-(4-(1H-1,2,4-triazol-5-yl)phenyl)-1H-benzo[d]imidazol-5-yl) (4,4-difluoropiperidin-1-yl)methanone, C-16 as an off white solid after Prep-HPLC purification.

C-15: (7.3% yield, m/z=410.2 [M+H]⁺.

C-16: (37.30% yield, m/z=409.1 [M+H]⁺).

Example 3. Synthesis of (3-(6-(1H-1,2,4-triazol-5-yl)pyridin-3-yl)-3H-[1,2,3]triazolo[4,5-b]pyridin-6-yl) (4,4-difluoropiperidin-1-yl)methanone (C-5) and (4,4-difluoropiperidin-1-yl)(3-(6-(1-methyl-1H-1,2,4-triazol-5-yl)pyridin-3-yl)-3H-[1,2,3]triazolo[4,5-b]pyridin-6-yl)methanone (C-8)

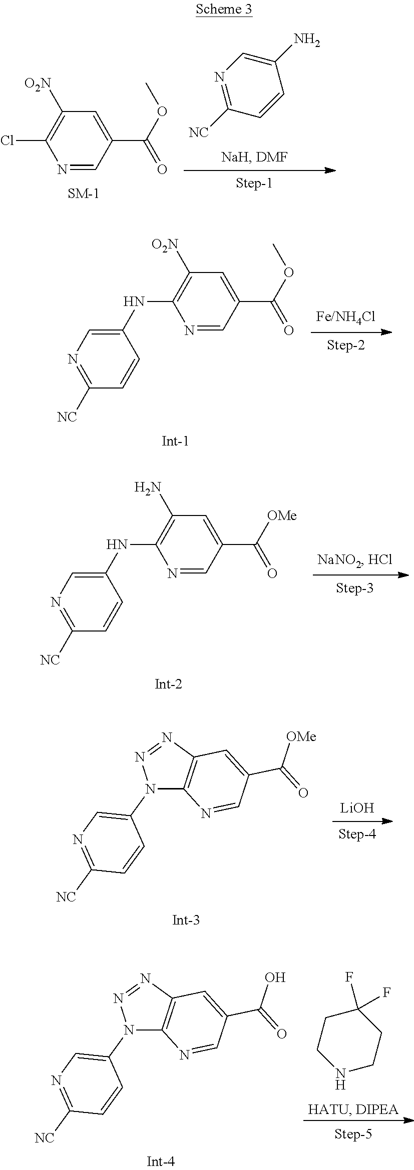

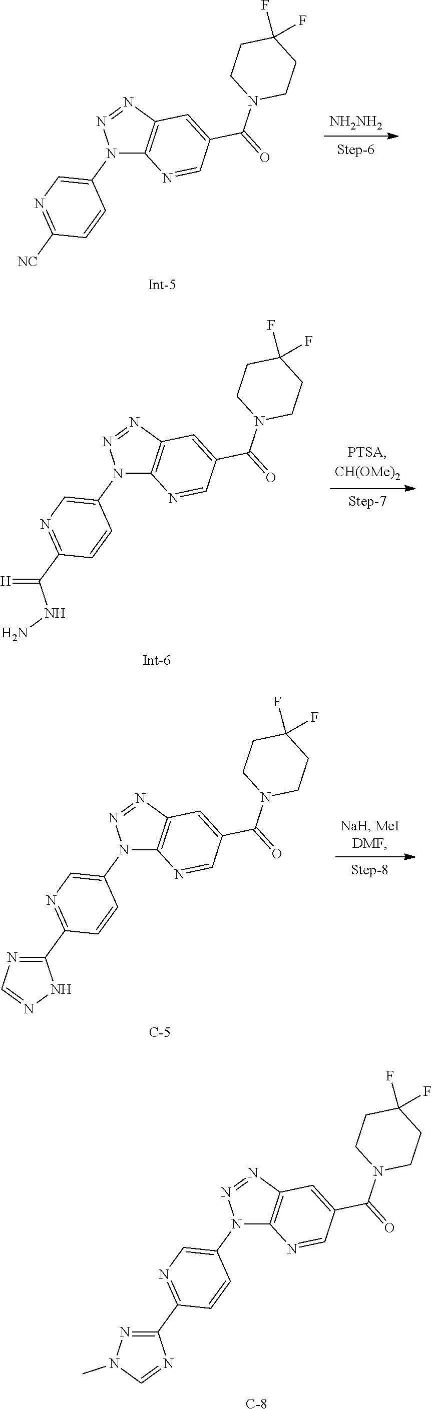

Step-1: Synthesis of methyl 6-((6-cyanopyridin-3-yl)amino)-5-nitronicotinate (Int-1), general procedure for S_{N}Ar reaction using NaH: To a stirred solution of 5-aminopicolinonitrile (880 mg, 7.39 mmol, 0.8 eq.) in DMF (40 mL) at 0° C., NaH (738 mg, 18.47 mmol, 2.0 eq.) was added portion wise at 0° C. and the reaction was stirred for 10 mins followed by addition of methyl 6-chloro-5-nitronicotinate (SM-1) (2 g, 9.23 mmol, 1.0 eq.) at RT. The resultant sealed reaction mixture was heated to 100° C. for 16 h. The progress of the reaction was monitored with TLC and LCMS. The reaction was quenched (ice-water, 20 mL) and extracted with ethyl acetate (2×50 mL). The combined extracts were dried over sodium sulphate, filtered and concentrated to afford methyl 6-((6-cyanopyridin-3-yl)amino)-5-nitronicotinate, Int-1 (2.2 g) as a brown oil. The crude was used in the next step without further purification TLC: 30% EtOAc/heptane (R_{f}: 0.45). MS: m/z=300.5 [M+H]⁺.

Step-2: Synthesis of methyl 5-amino-6-((6-cyanopyridin-3-yl)amino)nicotinate (Int-2) methyl 6-((6-cyanopyridin-3-yl)amino)-5-nitronicotinate (Int-1) (2.2 g, 7.35 mmol, 1.0 eq) was converted to methyl 5-amino-6-((6-cyanopyridin-3-yl)amino)nicotinate (Int-2) using the general procedure for reduction of nitro compounds using Fe/NH₄Cl. The crude was purified by combi-flash column chromatography using 55% EtOAc:heptane to afford methyl 5-amino-6-((6-cyanopyridin-3-yl)amino) nicotinate, Int-2 (1.2 g, 60%) as a yellow solid. TLC: 50% EtOAc (R_{f}: 0.35); MS: m/z=270.1 [M+H]⁺.

Step-3: Synthesis of methyl 3-(6-cyanopyridin-3-yl)-3H-[1,2,3]triazolo[4,5-b]pyridine-6-carboxylate (Int-3) (General procedure for triazolo formation with NaNO₂): To a solution of methyl 5-amino-6-((6-cyanopyridin-3-yl)amino) nicotinate (Int-2) (1.2 g, 4.46 mmol, 1 eq) dissolved in water (36 mL) was added NaNO₂ (992 mg, 13.38 mmol, 3 eq) at 0° C. followed by catalytic amount of conc. HCl (3.6 mL). The mixture was stirred at RT for 2 h. The progress of the reaction was monitored with TLC. The precipitated solid obtained in the reaction mixture was filtered, washed with water and dried. Trituration with Et₂O afforded methyl 3-(6-cyanopyridin-3-yl)-3H-[1, 2, 3]triazolo[4,5-b]pyridine-6-carboxylate, Int-3 (1.1 g) as a yellow solid. The crude was used in the next step without further purification. TLC: 70% EtOAc:heptane (R_{f}: 0.5); MS: m/z=280.2 [M+H]⁺.

Step-4: Synthesis of 3-(6-cyanopyridin-3-yl)-3H-[1,2,3]triazolo[4,5-b]pyridine-6-carboxylic acid (Int-4): methyl 3-(6-cyanopyridin-3-yl)-3H-[1, 2, 3]triazolo[4,5-b]pyridine-6-carboxylate, (Int-3) (1.1 g, 3.93 mmol, 1.0 eq) was converted to 3-(6-cyanopyridin-3-yl)-3H-[1,2,3]triazolo[4,5-b]pyridine-6-carboxylic acid using general procedure for ester hydrolysis with LiOH. The crude obtained was triturated with Et₂O to afford 3-(6-cyanopyridin-3-yl)-3H-[1,2,3]triazolo[4,5-b]pyridine-6-carboxylic acid, Int-4 (1.04 g, 95.69%) as an off-white solid. The crude was used in the next step without further purification TLC: 80% EtOAc (R_{f}: 0.2); MS: m/z=264.9 [M−H]⁻.

Step-5: Synthesis of 5-(6-(4,4-difluoropiperidine-1-carbonyl)-3H-[1,2,3]triazolo[4,5-b]pyridin-3-yl) picolinonitrile (Int-5): 3-(6-cyanopyridin-3-yl)-3H-[1,2,3]triazolo[4,5-b]pyridine-6-carboxylic acid (Int-4) (1 g, 3.76 mmol, 1.0 eq.) was converted to 5-(6-(4,4-difluoropiperidine-1-carbonyl)-3H-[1,2,3]triazolo[4,5-b]pyridin-3-yl) picolinonitrile (Int-5) using general procedure for acid-amine coupling with HATU/4,4-difluoropiperidine hydrochloride (886 mg, 5.63 mmol, 1.5 eq.). The crude obtained was triturated with Et₂O to afford 5-(6-(4,4-difluoropiperidine-1-carbonyl)-3H-[1,2,3]triazolo[4,5-b]pyridin-3-yl) picolinonitrile, Int-5 (500 mg, 36.23%) as an off-white solid. The crude was used in the next step without further purification. TLC: 5% MeOH/CH₂Cl₂ (R_{f}: 0.4); MS: m/z=370.25 [M+1]⁺.

Step-6: Synthesis of 5-(6-(4,4-difluoropiperidine-1-carbonyl)-3H-[1,2,3]triazolo[4,5-b]pyridin-3-yl) picolinimidohydrazide (Int-6) (General procedure for imidohydrazide formation using hydrazine): To a stirred solution of 5-(6-(4,4-difluoropiperidine-1-carbonyl)-3H-[1,2,3]triazolo[4,5-b]pyridin-3-yl) picolinonitrile (300 mg, 0.812 mmol, 1.0 eq) in ethanol (3 mL) was added hydrazine monohydrate (6 mL). The mixture was stirred at 60° C. for 1 h. The progress of the reaction was monitored with TLC. The solid obtained was filtered and dried. Trituration with Et₂O afforded 5-(6-(4,4-difluoropiperidine-1-carbonyl)-3H-[1,2,3]triazolo[4,5-b]pyridin-3-yl) picolinimidohydrazide, Int-6 (170 mg) as yellow solid. The crude obtained was used in the next step without further purification. TLC: 5% MeOH/CH₂Cl₂ (R_{f}: 0.25) MS: m/z=402.53 [M+H]⁺.

Step-7: Synthesis of (3-(6-(1H-1,2,4-triazol-5-yl)pyridin-3-yl)-3H-[1,2,3]triazolo[4,5-b]pyridin-6-yl) (4,4-difluoropiperidin-1-yl)methanone (C-5) (General procedure for triazole formation using triethylorthoformate): To a stirred solution of 5-(6-(4,4-difluoropiperidine-1-carbonyl)-3H-[1,2,3]triazolo[4,5-b]pyridin-3-yl) picolinimidohydrazide (Int-6) (170 mg, 0.423 mmol, 1.0 eq) in 1,4-dioxane (5 mL), was added triethylorthoformate (0.251 mL, 2.11 mmol, 5.0 eq) and p-toluenesulfonic acid monohydrate (14.5 mg, 0.086 mmol, 0.2 eq). The mixture was stirred at 100° C. for 16 h. The progress of the reaction was monitored with TLC/LCMS, After consumption of starting material, the mixture was quenched with saturated NaHCO₃ (10 mL). The solution was extracted with EtOAc (2×20 mL) and the organic extracts washed with water (5 mL) and brine (5 mL). The organic layer was dried over Na₂SO₄, filtered and concentrated. The crude was purified by combi-flash column chromatography using 5% MeOH:DCM to afford (3-(6-(1H-1,2,4-triazol-5-yl)pyridin-3-yl)-3H-[1,2,3]triazolo[4,5-b]pyridin-6-yl)(4,4-difluoropiperidin-1-yl)methanone (C-5) (130 mg, 74.71%) as off white solid. TLC: 5% MeOH/CH₂Cl₂ (R_{f}: 0.2); LCMS: 98.33%, MS: m/z=412.1 [M+H]⁺.

Step-7: Synthesis of (4,4-difluoropiperidin-1-yl)(3-(6-(1-methyl-1H-1,2,4-triazol-5-yl) pyridin-3-yl)-3H-[1,2,3]triazolo[4,5-b]pyridin-6-yl) methanone (C-8) (General procedure for methylation of amine using NaH): To a stirred solution of (3-(6-(1H-1,2,4-triazol-5-yl)pyridin-3-yl)-3H-[1,2,3]triazolo[4,5-b]pyridin-6-yl)(4,4-difluoropiperidin-1-yl)methanone (C-8) (100 mg, 0.243 mmol, 1 eq) in DMF (5 mL), NaH (60% in mineral oil) (19 mg, 0.486 mmol, 2 eq) was added at 0° C., slowly brought to RT and stirred for 1 h. Mel (0.05 mL, 0.729 mmol, 3 eq) was added and then the resulting mixture was stirred for 1 h. The reaction was monitored by crude LCMS/TLC; after complete consumption of the starting material, the reaction mixture was quenched with sat. NH₄Cl (10 ml) and extracted with EtOAc (2×20 mL). Combined organic extracts were washed with brine (10 mL), dried over sodium sulfate, filtered and concentrated in vacuo to obtain the crude. The crude was purified by combi-flash column chromatography using 5% MeOH:DCM to afford (4,4-difluoropiperidin-1-yl) (3-(6-(1-methyl-1H-1,2,4-triazol-5-yl) pyridin-3-yl)-3H-[1,2,3]triazolo[4,5-b]pyridin-6-yl) methanone (10 mg, 9.70%) ((C-8) as an off-white solid. TLC: 10% MeOH:DCM (R_{f}: 0.35). LCMS: 96.35%, m/z=426.2[M+H]⁺.

### Example 4. Synthesis of (3-(4-(1H-1,2,4-triazol-5-yl)phenyl)-3H-[1,2,3]triazolo[4,5-b]pyridin-6-yl) (4,4-difluoropiperidin-1-yl)methanone (C-7)

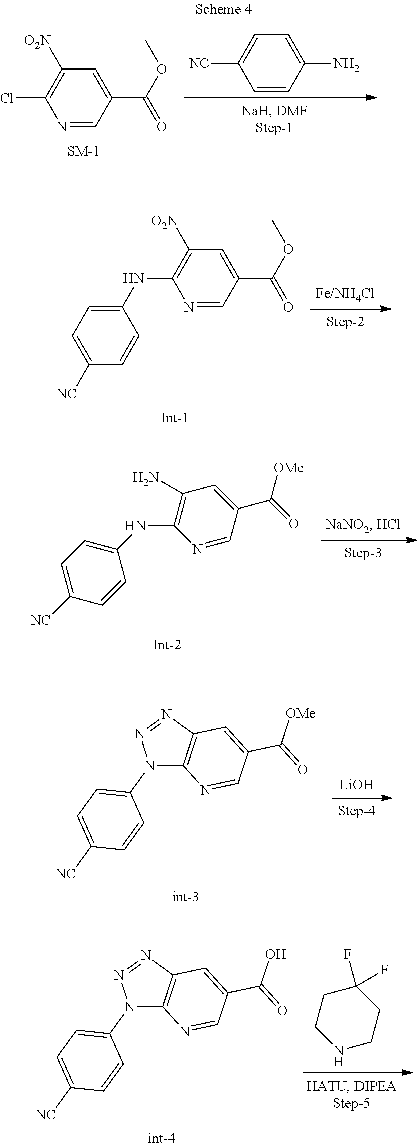

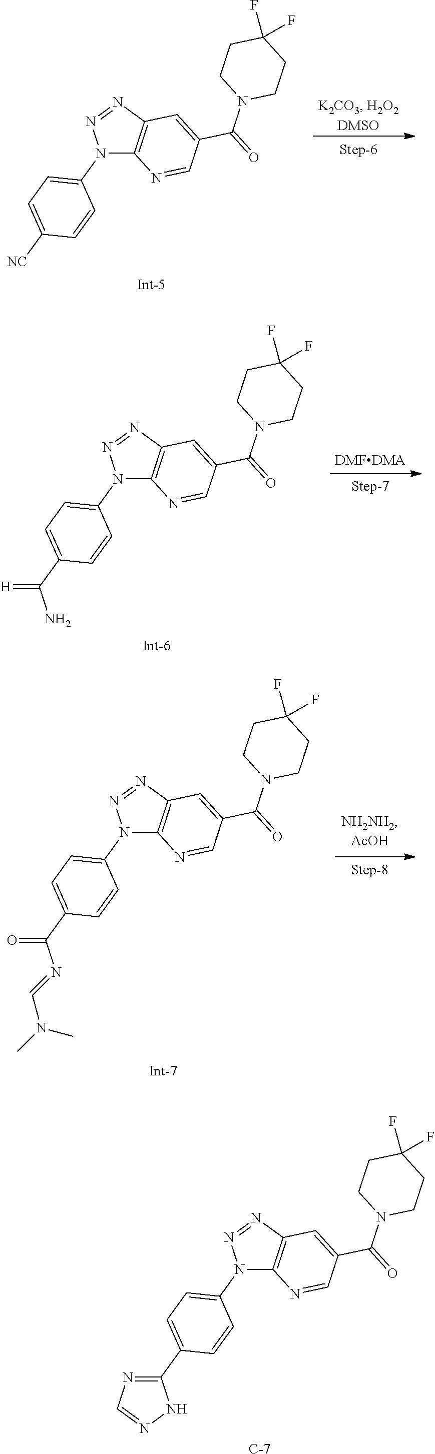

Step-1: methyl 6-((4-cyanophenyl)amino)-5-nitronicotinate (Int-1): methyl 6-chloro-5-nitronicotinate (SM-1) (2.6 g, 12 mmol, 1.0 eq), was converted to methyl 6-((4-cyanophenyl)amino)-5-nitronicotinate (Int-1) using general procedure for S_{N}Ar reaction using NaH (as described above) to afford methyl 6-((4-cyanophenyl)amino)-5-nitronicotinate, Int-1 (1.5 g, 42%) as a brown oil. The crude was used in the next step without further purification TLC: 30% EtOAc/heptane (R_{f}: 0.45). MS: m/z=299.2[M+H]⁺.

Step-2: Synthesis of methyl 5-amino-6-((4-cyanophenyl)amino)nicotinate (Int-2): methyl 6-((4-cyanophenyl) amino)-5-nitronicotinate (Int-1) (1.5 g, 5.03 mmol, 1.0 eq) was converted to methyl 5-amino-6-((4-cyanophenyl)amino)nicotinate (Int-2) using the general procedure for reduction of nitro compounds using Fe/NH₄Cl. The crude was purified by combi-flash column chromatography using 40% EtOAc:heptane to afford methyl 5-amino-6-((4-cyanophenyl)amino)nicotinate, Int-2 (1.21 g, 89.55%) as a yellow solid. TLC: 50% EtOAc (R_{f}: 0.35); LCMS: 87.63% MS: m/z=269.19 [M+H]⁺.

Step-3: Synthesis of methyl 3-(4-cyanophenyl)-3H-[1,2,3]triazolo[4,5-b]pyridine-6-carboxylate (Int-3): methyl 5-amino-6-((4-cyanophenyl)amino)nicotinate (Int-2) (1.1 g, 4.10 mmol, 1 eq) was converted to methyl 3-(4-cyanophenyl)-3H-[1,2,3]triazolo[4,5-b]pyridine-6-carboxylate using the general procedure for triazole formation using NaNO₂/conc. HCl to afford methyl 3-(4-cyanophenyl)-3H-[1,2,3]triazolo[4,5-b]pyridine-6-carboxylate, Int-3 (1 g). The crude was used in the next step without further purification. TLC: 60% EtOAc:heptane (R_{f}: 0.5); MS: m/z=280.17 [M+H]*.

Step-4: Synthesis of 3-(4-cyanophenyl)-3H-[1,2,3]triazolo[4,5-b]pyridine-6-carboxylic acid (Int-4): methyl 3-(4-cyanophenyl)-3H-[1,2,3]triazolo[4,5-b]pyridine-6-carboxylate, (Int-3) (1 g, 3.58 mmol, 1.0 eq) was converted to 3-(4-cyanophenyl)-3H-[1,2,3]triazolo[4,5-b]pyridine-6-carboxylic acid (Int-4) using the general procedure for ester hydrolysis with LiOH. The crude obtained was triturated with Et₂O to afford 3-(4-cyanophenyl)-3H-[1,2,3]triazolo[4,5-b]pyridine-6-carboxylic acid, Int-4 (900 mg, 94.83%) as an off-white solid. The crude was used in the next step without further purification TLC: 80% EtOAc:heptane (R_{f}: 0.2); MS: m/z=266.1 [M+H]⁺.

Step-5: Synthesis of 4-(6-(4,4-difluoropiperidine-1-carbonyl)-3H-[1,2,3]triazolo[4,5-b]pyridin-3-yl) benzonitrile (Int-5): 3-(4-cyanophenyl)-3H-[1,2,3]triazolo[4,5-b]pyridine-6-carboxylic acid (Int-4) (900 mg, 3.39 mmol, 1.0 eq.) was converted to 4-(6-(4,4-difluoropiperidine-1-carbonyl)-3H-[1,2,3]triazolo[4,5-b]pyridin-3-yl)benzonitrile (Int-5) using general procedure for acid-amine coupling with HATU/4,4-difluoropiperidine hydrochloride (886 mg, 5.63 mmol, 1.5 eq.). The crude obtained was triturated with n-pentane to afford 4-(6-(4,4-difluoropiperidine-1-carbonyl)-3H-[1,2,3]triazolo[4,5-b]pyridin-3-yl)benzonitrile, Int-5 (1 g, 80.64%) as off white solid. The crude was used in the next step without further purification. TLC: 5% MeOH/CH₂Cl₂ (R_{f}: 0.5); LCMS: 86.30%, MS: m/z=369.2 [M+H].

Step-6: Synthesis of 4-(6-(4,4-difluoropiperidine-1-carbonyl)-3H-[1,2,3]triazolo[4,5-b]pyridin-3-yl) benzamide (Int-6), general procedure for amide formation using H₂O₂: To a stirred solution of 4-(6-(4,4-difluoropiperidine-1-carbonyl)-3H-[1,2,3]triazolo[4,5-b]pyridin-3-yl)benzonitrile (Int-5) (500 mg, 1.4 mmol, 1 eq) in DMSO (10 ml) cooled in an ice bath was added H₂O₂ (30%, 0.33 ml, 2.8 mmol, 2.0 eq) and potassium carbonate (202 mg, 1.40 mmol, 1.0 eq). The mixture was then allowed to warm up to room temperature and stirred at 60° C. for 2 h. The progress of the reaction was monitored with TLC. The solid obtained was filtered and dried. Trituration with Et₂O afforded 4-(6-(4,4-difluoropiperidine-1-carbonyl)-3H-[1,2,3]triazolo[4,5-b]pyridin-3-yl) benzamide, Int-6 (400 mg) as a yellow solid. The crude obtained was used in the next step without further purification. TLC: 80% EtOAc:heptane (R_{f}: 0.35) MS: m/z=387.02 [M+H]⁺.

Step-7: Synthesis of (E)-4-(6-(4,4-difluoropiperidine-1-carbonyl)-3H-[1,2,3]triazolo[4,5-b]pyridin-3-yl)-N-((dimethylamino)methylene)benzamide (Int-7) (General procedure for enamine formation with DMF-DMA): A solution of 4-(6-(4,4-difluoropiperidine-1-carbonyl)-3H-[1,2,3]triazolo[4,5-b]pyridin-3-yl)benzamide (Int-6) (400 mg, 1.04 mmol, 1.0 eq) and N,N-dimethylformamide dimethyl acetal (DMF-DMA) (10 mL) were stirred at 100° C. under nitrogen atmosphere for 1 h. The progress of the reaction was monitored with TLC. The mixture was evaporated under reduced pressure, crude obtained was triturated with Et₂O to afford (E)-4-(6-(4,4-difluoropiperidine-1-carbonyl)-3H-[1,2,3]triazolo[4,5-b]pyridin-3-yl)-N—((dimethylamino)methylene)benzamide, Int-7 (350 mg, 76.75%) as pale yellow solid. The crude obtained was used in the next step without further purification. TLC: 5% MeOH/CH₂Cl₂ (R_{f}: 0.35) LCMS: 95.34%, MS: m/z=442.3 [M+H]⁺.

Step-8:(3-(4-(1H-1,2,4-triazol-5-yl)phenyl)-3H-[1,2,3]triazolo[4,5-b]pyridin-6-yl)(4,4-difluoropiperidin-1-yl) methanone (MH-DH-613) (General procedure for triazole formation using hydrazine acetate): To a stirred solution of (E)-4-(6-(4,4-difluoropiperidine-1-carbonyl)-3H-[1, 2, 3]triazolo[4, 5-b]pyridin-3-yl)-N-((dimethylamino)methylene) benzamide, (Int-7) (100 mg, 0.226 mmol, 1.0 eq) dissolved in acetic acid (0.5 mL) was added hydrazine acetate (104 mg, 1.13 mmol, 5.0 eq) at room temperature. The resultant mixture was stirred at 95° C. for 2 h. The progress of the reaction was monitored with TLC. After consumption of starting material, the mixture was quenched with saturated NaHCO₃ (10 mL). The solution was extracted with EtOAc (2×20 mL) and washed with water (5 mL) and brine (5 mL). The organic layer was dried over Na₂SO₄, filtered and concentrated. The crude was purified by combi-flash column chromatography using 5% MeOH:DCM to afford (3-(4-(1H-1,2,4-triazol-5-yl) phenyl)-3H-[1,2,3]triazolo[4,5-b]pyridin-6-yl) (4,4-difluoropiperidin-1-yl) methanone (C-7) (65 mg, 67.39%) as off white solid. TLC: 5% MeOH/CH2Cl2 (R_{f}: 0.35) LCMS: 97.21%, MS: m/z=411.1 [M+H]⁺.

### Example 5. Synthesis of (1-(6-(1H-1,2,4-triazol-5-yl)pyridin-3-yl)-1H-benzo[d][1,2,3]triazol-5-yl) (4,4-difluoropiperidin-1-yl)methanone (C-9)

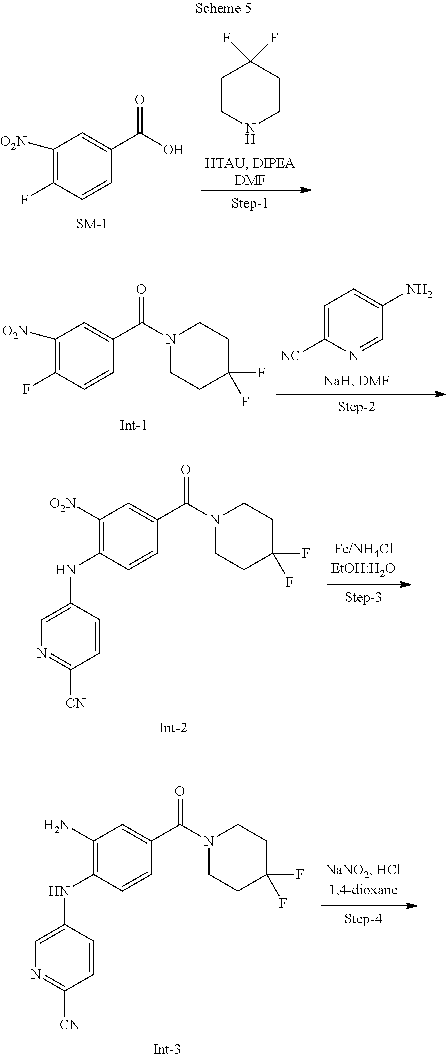

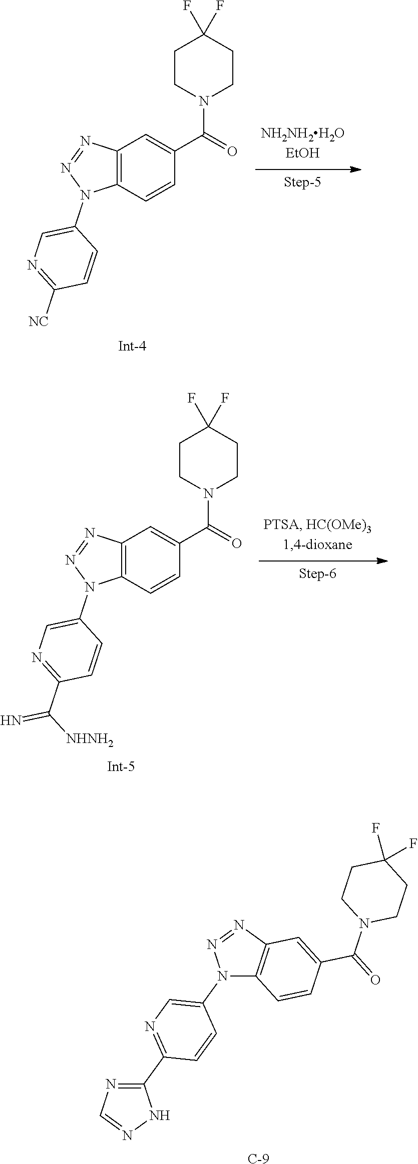

Step-1: Synthesis of (4,4-difluoropiperidin-1-yl)(4-fluoro-3-nitrophenyl)methanone (Int-1): 4-fluoro-3-nitrobenzoic acid, SM-1 (2 g, 10.80 mmol, 1.0 eq.) was converted to (4,4-difluoropiperidin-1-yl)(4-fluoro-3-nitrophenyl)methanone (Int-1) using general procedure for acid-amine coupling with HATU/4,4-difluoropiperidine hydrochloride (2.04 g, 12.97 mmol, 1.2 eq). The crude solid obtained was filtered, washed with diethyl ether (2×5 mL) and dried under reduced pressure to afford (4,4-difluoropiperidin-1-yl)(4-fluoro-3-nitrophenyl)methanone, Int-1 (1.2 g, 38.50%) as off white solid. TLC: 50% EtOAc/Heptane (R_{f}: 0.40) MS: m/z=289.2 [M+H]⁺.

Step-2: Synthesis of 5-((4-(4,4-difluoropiperidine-1-carbonyl)-2-nitrophenyl) amino) picolinonitrile (Int-2): (4,4-difluoropiperidin-1-yl)(4-fluoro-3-nitrophenyl)methanone, (Int-1) (1.2 g, 4.16 mmol, 1.0 eq) was converted to 5-((4-(4,4-difluoropiperidine-1-carbonyl)-2-nitrophenyl) amino)picolinonitrile (Int-2) using general procedure for S_{N}Ar reaction using NaH. The crude was purified by combi-flash column chromatography using 50% EtOAc:heptane to afford methyl 5-((4-(4,4-difluoropiperidine-1-carbonyl)-2-nitrophenyl) amino) picolinonitrile, Int-2 (900 mg, 55.80%) as a pale brown oil. TLC: 40% EA:Hex (R_{f}: 0.3). MS: m/z=388.2 [M+H]⁺.

Step-3: Synthesis of 5-((2-amino-4-(4,4-difluoropiperidine-1-carbonyl) phenyl) amino) picolinonitrile (Int-3): methyl 4-((4-(4,4-difluoropiperidine-1-carbonyl)-2-nitrophenyl) amino) benzoate (Int-2) (800 mg, 3.34 mmol, 1.0 eq) was converted to 5-((2-amino-4-(4,4-difluoropiperidine-1-carbonyl)phenyl)amino)picolinonitrile (Int-3) using the general procedure for reduction of nitro compounds with Fe/NH₄Cl. The crude product was purified by combi-flash column chromatography using 50% EtOAc:heptane to afford 5-((2-amino-4-(4,4-difluoropiperidine-1-carbonyl) phenyl)amino)picolinonitrile, Int-3 (620 mg, 84%) as a yellow solid. TLC: 60% EtOAc:heptane (R_{f}: 0.3); MS: m/z=358.3 [M+H]⁺.

Step-4: Synthesis of 5-(5-(4,4-difluoropiperidine-1-carbonyl)-1H-benzo[d][1,2,3]triazol-1-yl) picolinonitrile (Int-4): 5-((2-amino-4-(4,4-difluoropiperidine-1-carbonyl) phenyl) amino) picolinonitrile (Int-3) (500 mg, 1.40 mmol, 1 eq) was converted to 5-(5-(4,4-difluoropiperidine-1-carbonyl)-1H-benzo[d][1,2,3]triazol-1-yl)picolinonitrile using general procedure for triazolo formation to afford 5-(5-(4,4-difluoropiperidine-1-carbonyl)-1H-benzo[d][1,2,3]triazol-1-yl) picolinonitrile, Int-4 (300 mg, 58.30%) as a yellow solid. TLC: 70% EtOAc:heptane (R_{f}: 0.4); MS: m/z=369.2 [M+H]⁺.

Step-5: Synthesis of 5-(5-(4,4-difluoropiperidine-1-carbonyl)-1H-benzo[d][1,2,3]triazol-1-yl) picolinimidohydrazide (Int-5): 5-(5-(4,4-difluoropiperidine-1-carbonyl)-1H-benzo[d][1,2,3]triazol-1-yl)picolinonitrile (300 mg, 0.814 mmol, 1.0 eq) was converted to 5-(5-(4,4-difluoropiperidine-1-carbonyl)-1H-benzo[d][1,2,3]triazol-1-yl)picolinimidohydrazide, (Int-5), using the general procedure for imidohydrazide formation using hydrazine to afford 5-(5-(4,4-difluoropiperidine-1-carbonyl)-1H-benzo[d][1,2,3]triazol-1-yl)picolinimidohydrazide, Int-5 (180 mg, 66.40%) as pale yellow solid. TLC: 100% EtOAc (R_{f}: 0.25) MS: m/z=401.56 [M+H]⁺.

Step-7: Synthesis of (1-(6-(1H-1,2,4-triazol-5-yl)pyridin-3-yl)-1H-benzo[d][1,2,3]triazol-5-yl) (4,4-difluoropiperidin-1-yl)methanone (C-9): 5-(5-(4,4-difluoropiperidine-1-carbonyl)-1H-benzo[d][1,2,3]triazol-1-yl)picolinimidohydrazide (Int-5) (180 mg, 0.450 mmol, 1.0 eq) was converted to (1-(6-(1H-1,2,4-triazol-5-yl)pyridin-3-yl)-1H-benzo[d][1,2,3]triazol-5-yl) (4,4-difluoropiperidin-1-yl) methanone (C-9) using the general procedure for triazole formation with triethylorthoformate to afford (1-(6-(1H-1,2,4-triazol-5-yl)pyridin-3-yl)-1H-benzo[d][1,2,3]triazol-5-yl) (4,4-difluoropiperidin-1-yl)methanone (C-9) (60 mg, 32.6%) as off white solid. TLC: 10% MeOH/CH₂Cl₂ (R_{f}: 0.2); LCMS: 98.33%, MS: m/z=411.5 [M+H]⁺.

### Example 6. Synthesis of (1-(4-(1H-1,2,4-triazol-5-yl)phenyl)-1H-benzo[d][1,2,3]triazol-5-yl) (4,4-difluoropiperidin-1-yl)methanone (C-10)

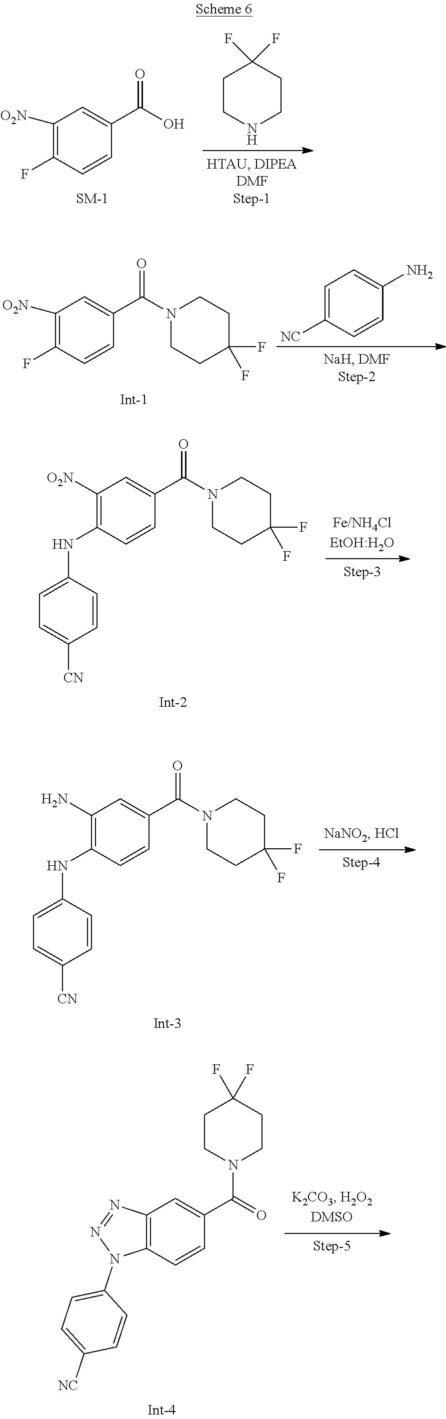

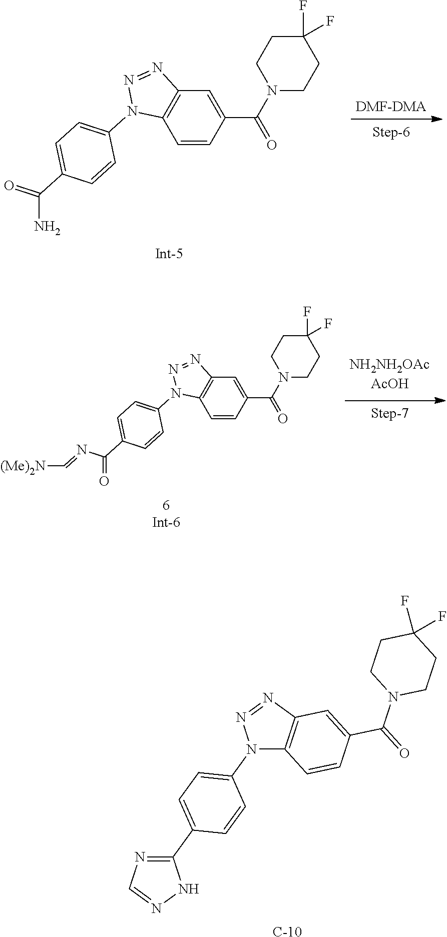

Step-1: Synthesis of (4,4-difluoropiperidin-1-yl)(4-fluoro-3-nitrophenyl)methanone (Int-1): Previously described in the synthesis of C-9.

Step-2: Synthesis of 4-((4-(4,4-difluoropiperidine-1-carbonyl)-2-nitrophenyl) amino) benzonitrile (Int-2): (4,4-difluoropiperidin-1-yl)(4-fluoro-3-nitrophenyl)methanone, (Int-1) (1 g, 3.47 mmol, 1.0 eq) was converted 4-((4-(4,4-difluoropiperidine-1-carbonyl)-2-nitrophenyl) amino) benzonitrile (Int-2) using general procedure for S_{N}Ar reaction with NaH. The crude was purified by combi-flash column chromatography using 50% EtOAc:heptane to afford 4-((4-(4,4-difluoropiperidine-1-carbonyl)-2-nitrophenyl)amino)benzonitrile, Int-2 (850 mg, 63.40%) as pale brown oil. TLC: 40% EA:Hex (R_{f}: 0.3). MS: m/z=387.4 [M+H]⁺.

Step-3: Synthesis of 4-((2-amino-4-(4,4-difluoropiperidine-1-carbonyl)phenyl) amino) benzonitrile (Int-3):4-((4-(4,4-difluoropiperidine-1-carbonyl)-2-nitrophenyl) amino) benzonitrile (Int-2) (850 mg, 3.34 mmol, 1.0 eq) was converted to 4-((2-amino-4-(4,4-difluoropiperidine-1-carbonyl)phenyl)amino)benzonitrile (Int-3) using the general procedure for reduction of nitro compounds with Fe/NH₄Cl. The crude product was purified by combi-flash column chromatography using 50% EtOAc:heptane to afford 4-((2-amino-4-(4,4-difluoropiperidine-1-carbonyl)phenyl)amino)benzonitrile, Int-3 (750 mg, 95.70%) as yellow solid. TLC: 60% EtOAc:heptane (R_{f}: 0.3); MS: m/z=357.2 [M+H]⁺.

Step-4: Synthesis of 5-(5-(4,4-difluoropiperidine-1-carbonyl)-1H-benzo[d][1,2,3]triazol-1-yl) picolinonitrile (Int-4): 4-((2-amino-4-(4,4-difluoropiperidine-1-carbonyl)phenyl) amino) benzonitrile (Int-3) (370 mg, 1.04 mmol, 1 eq) was converted to 4-(5-(4,4-difluoropiperidine-1-carbonyl)-1H-benzo[d][1,2,3]triazol-1-yl)benzonitrile (Int-4) using general procedure fortriazolo formation with NaNO₂ to afford 4-(5-(4,4-difluoropiperidine-1-carbonyl)-1H-benzo[d][1,2,3]triazol-1-yl)benzonitrile, Int-4 (310 mg, 81.50%) as a yellow solid. TLC: 70% EtOAc:heptane (R_{f}: 0.4); MS: m/z=368.2 [M+H]⁺.

Step-5: Synthesis of 4-(5-(4,4-difluoropiperidine-1-carbonyl)-1H-benzo[d][1,2,3]triazol-1-yl) benzamide (Int-5): 4-(5-(4,4-difluoropiperidine-1-carbonyl)-1H-benzo[d][1,2,3]triazol-1-yl)benzonitrile (Int-4) (250 mg, 0.680 mmol, 1.0 eq) was converted to 4-(5-(4,4-difluoropiperidine-1-carbonyl)-1H-benzo[d][1,2,3]triazol-1-yl)benzamide using general procedure for amide formation with H₂O₂ to afford 4-(5-(4,4-difluoropiperidine-1-carbonyl)-1H-benzo[d][1,2,3]triazol-1-yl)benzamide (180 mg, 69%) Int-5, as pale yellow solid TLC: 80% EtOAc:heptane (R_{f}: 0.35) MS: m/z=386.2 [M+H]⁺.

Step-6: Synthesis of (E)-4-(5-(4,4-difluoropiperidine-1-carbonyl)-1H-benzo[d][1,2,3]triazol-1-yl)-N-((dimethylamino)methylene)benzamide (Int-6): 4-(5-(4,4-difluoropiperidine-1-carbonyl)-1H-benzo[d][1,2,3]triazol-1-yl)benzamide (Int-5) (180 mg, 1.04 mmol, 1.0 eq) was converted to (E)-4-(5-(4,4-difluoropiperidine-1-carbonyl)-1H-benzo[d][1,2,3]triazol-1-yl)-N-((dimethylamino)methylene) benzamide (Int-6) using the general procedure for enamine formation with DMF-DMA. The crude obtained was triturated with Et₂O to afford (E)-4-(5-(4, 4-difluoropiperidine-1-carbonyl)-1H-benzo[d][1,2,3]triazol-1-yl)-N-((dimethylamino)methylene) benzamide (150 mg) as a pale brown solid. TLC: 80% EtOAc (R_{f}: 0.5) MS: m/z=441.2 [M+H]⁺.

Step-7: Synthesis of (1-(4-(1H-1,2,4-triazol-5-yl)phenyl)-1H-benzo[d][1,2,3]triazol-5-yl)(4,4-difluoropiperidin-1-yl)methanone (C-10): (E)-4-(5-(4,4-difluoropiperidine-1-carbonyl)-1H-benzo[d][1,2,3]triazol-1-yl)-N-((dimethylamino)methylene) benzamide, (Int-6) (150 mg) was converted to (1-(4-(1H-1,2,4-triazol-5-yl)phenyl)-1H-benzo[d][1,2,3]triazol-5-yl)(4,4-difluoropiperidin-1-yl) methanone using general procedure for triazole formation using hydrazine acetate. The crude was purified by combi-flash column chromatography using 5% MeOH:DCM to afford (1-(4-(1H-1, 2, 4-triazol-5-yl)phenyl)-1H-benzo[d][1, 2, 3]triazol-5-yl) (4, 4-difluoropiperidin-1-yl) methanone (C-10) (75 mg, 54%) as off white solid. TLC: 5% MeOH/CH₂Cl₂ (R_{f}: 0.25) LCMS: 92.19%, MS: m/z=410.1 [M+H]⁺.

### Example 7. Synthesis of 4-(5-(4,4-difluoropiperidine-1-carbonyl)-1H-benzo[d][1,2,3]triazol-1-yl) benzoic acid (C-11)

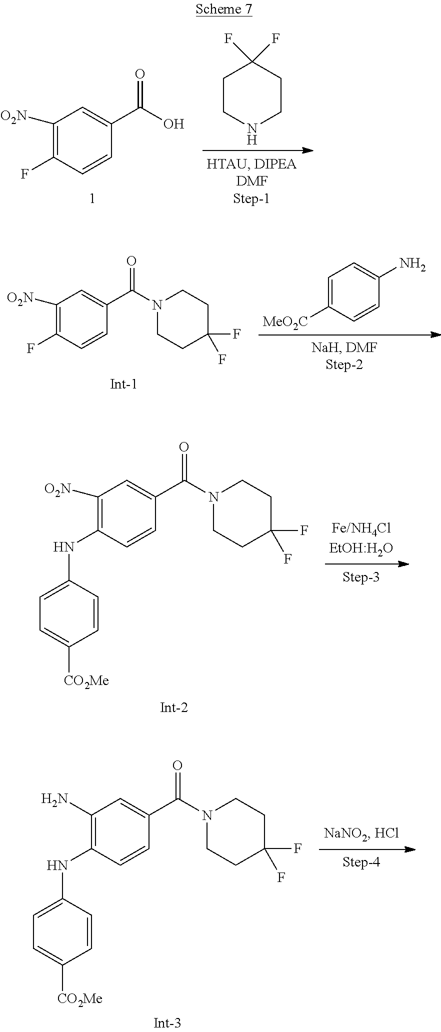 [SMILES: O=C(O)c1ccc(F)c([N+](=O)[O-])c1 ; FC1(F)CCNCC1 ; O=C(c1ccc(F)c([N+](=O)[O-])c1)N1CCC(F)(F)CC1 ; COC(=O)c1ccc(N)cc1 ; COC(=O)c1ccc(Nc2ccc(C(=O)N3CCC(F)(F)CC3)cc2[N+](=O)[O-])cc1 ; COC(=O)c1ccc(Nc2ccc(C(=O)N3CCC(F)(F)CC3)cc2N)cc1]

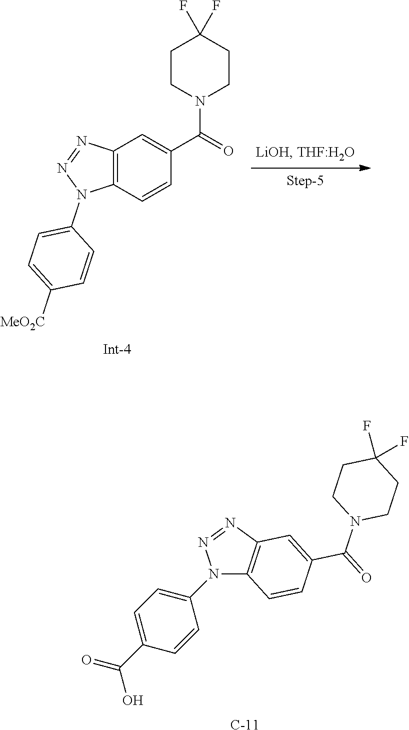

Step-1: Int-1: Previously described in the synthesis of C-9.

Step-2: Synthesis of methyl 4-((4-(4,4-difluoropiperidine-1-carbonyl)-2-nitrophenyl) amino) benzoate (Int-2): 4,4-difluoropiperidin-1-yl)(4-fluoro-3-nitrophenyl)methanone, (Int-1) (2 g, 6.94 mmol, 1.0 eq) was converted to methyl 4-((4-(4,4-difluoropiperidine-1-carbonyl)-2-nitrophenyl) amino) benzoate (Int-2) using general procedure for S_{N}Ar reaction with NaH. The crude was purified by combi-flash column chromatography using 50% EtOAc:heptane to afford methyl 4-((4-(4,4-difluoropiperidine-1-carbonyl)-2-nitrophenyl) amino) benzoate, Int-2 (1.4 g, 48.10%) as pale brown oil. TLC: 40% EA:Hex (R_{f}: 0.3). MS: m/z=420.40 [M+H]⁺.

Step-3: Synthesis of methyl 4-((2-amino-4-(4,4-difluoropiperidine-1-carbonyl) phenyl) amino) benzoate (Int-3): methyl 4-((4-(4,4-difluoropiperidine-1-carbonyl)-2-nitrophenyl) amino) benzoate (Int-2) (1.4, 3.34 mmol, 1.0 eq) was converted to methyl 4-((3-amino-5-(4,4-difluoropiperidine-1-carbonyl)pyridin-2-yl)amino)benzoate (Int-3) using the general procedure for reduction of nitro compounds using Fe/NH₄Cl. The crude product was purified by combi-flash column chromatography using 50% EtOAc:heptane to afford methyl 4-((3-amino-5-(4,4-difluoropiperidine-1-carbonyl)pyridin-2-yl)amino)benzoate, Int-3 (705 mg, 54.26%) as a yellow solid. TLC: 60% EtOAc:heptane (R_{f}: 0.3); MS: m/z=390.2 [M+H]⁺.

Step-4: Synthesis of methyl 4-(5-(4,4-difluoropiperidine-1-carbonyl)-1H-benzo[d][1,2,3]triazol-1-yl)benzoate (Int-4): methyl 4-((2-amino-4-(4,4-difluoropiperidine-1-carbonyl) phenyl) amino) benzoate (Int-3) (250 mg, 0.642 mmol, 1 eq) was converted to methyl 4-(5-(4,4-difluoropiperidine-1-carbonyl)-1H-benzo[d][1,2,3]triazol-1-yl) benzoate using general procedure for triazolo formation to afford methyl 4-(5-(4,4-difluoropiperidine-1-carbonyl)-1H-benzo[d][1,2,3]triazol-1-yl)benzoate, Int-4 (200 mg, 77.8%) as a yellow solid. TLC: 70% EtOAc:heptane (R_{f}: 0.4); MS: m/z=401.2 [M+H]⁺.

Step-5: Synthesis of 4-(5-(4,4-difluoropiperidine-1-carbonyl)-1H-benzo[d][1,2,3]triazol-1-yl) benzoic acid (C-11): methyl 4-(5-(4,4-difluoropiperidine-1-carbonyl)-1H-benzo[d][1,2,3]triazol-1-yl)benzoate, (Int-4) (200 mg, 0.499 mmol, 1 eq) was converted to 4-(5-(4,4-difluoropiperidine-1-carbonyl)-1H-benzo[d][1,2,3]triazol-1-yl) benzoic acid (C-11) using general procedure for hydrolysis with LiOH to afford 4-(5-(4,4-difluoropiperidine-1-carbonyl)-1H-benzo[d][1,2,3]triazol-1-yl)benzoic acid (C-11) (35 mg, 24.47%) as an off white solid. TLC: 80% EtOAc:heptane (R_{f}: 0.2); LCMS: 95.63%, MS: m/z=387.1 [M+H]⁺.

### Example 8. Synthesis of Compounds C-2 and C-14

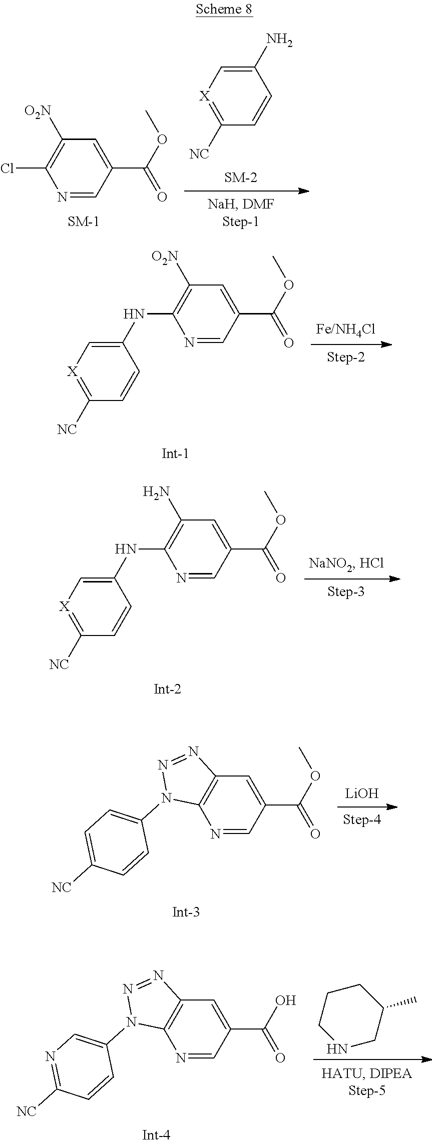

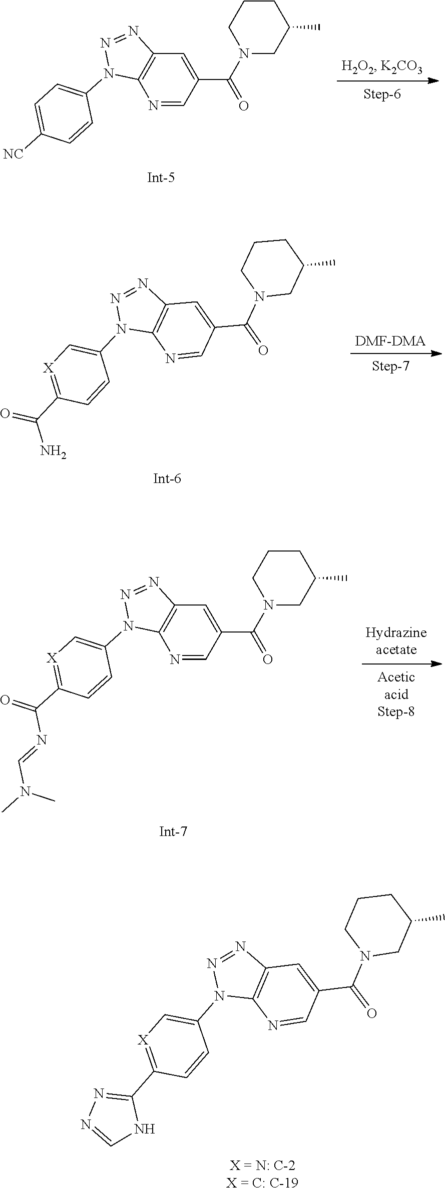

Step-1: Synthesis of Int-5a and Int-5b was accomplished using the general procedure for Ullmann coupling as described above; Int-5a (m/z=401.1, [M+H]⁺), Int-5b (60.19%, m/z=442.2, [M+H]⁺).

Step-2A: Synthesis of 4-(5-(4,4-difluoropiperidine-1-carbonyl)-1H-pyrazolo[3,4-b]pyridin-1-yl) benzoic acid, C-2: Int-5a was hydrolyzed using the general procedure for ester hydrolysis with LiOH to C-2 (31%, m/z=385.25, [M−H]⁺) as an off white solid.

Step-2B: Synthesis of 4-(5-(4,4-difluoropiperidine-1-carbonyl)-1H-indazol-1-yl) benzoic acid, C-14 and 4-(5-(4,4-difluoropiperidine-1-carbonyl)-2H-indazol-2-yl) benzoic acid: To a stirred solution of tert-butyl 4-(5-(4,4-difluoropiperidine-1-carbonyl)-1H-indazol-1-yl)benzoate (Int-5b) (180 mg, 0.407 mmol, 1 eq) in DCM (6 mL), TFA (2 mL) was added at 0° C. The resulting reaction mixture was stirred at room temperature for 16 h. The reaction was monitored by crude LCMS/TLC; upon completion, the reaction mixture was concentrated in vacuo to afford crude. Crude was purified by prep-HPLC to afford 4-(5-(4,4-difluoropiperidine-1-carbonyl)-1H-indazol-1-yl) benzoic acid, C-14 (25.5%, m/z=386.2, [M+H]⁺) as off white solid and 4-(5-(4,4-difluoropiperidine-1-carbonyl)-2H-indazol-2-yl) benzoic acid (11.5%, m/z=386.2, [M+H]⁺) as off white solid.

Example 9. Synthesis of (S)-(3-(4-(4H-1,2,4-triazol-3-yl)phenyl)-3H-[1,2,3]triazolo[4,5-b]pyridin-6-yl)(3-methylpiperidin-1-yl)methanone (C-18)/(S)-(3-(6-(4H-1,2,4-triazol-3-yl)pyridin-3-yl)-3H-[1,2,3]triazolo[4,5-b]pyridin-6-yl)(3-methylpiperidin-1-yl)methanone (C-19)

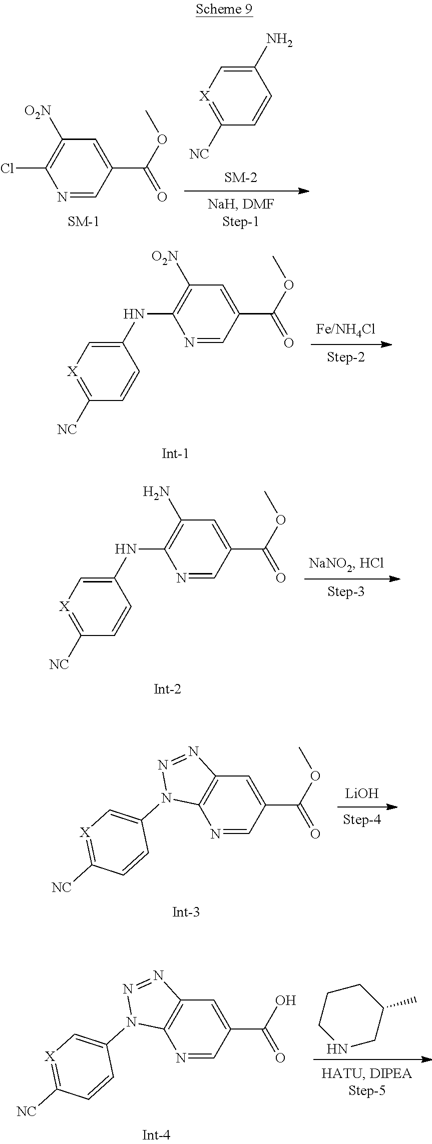

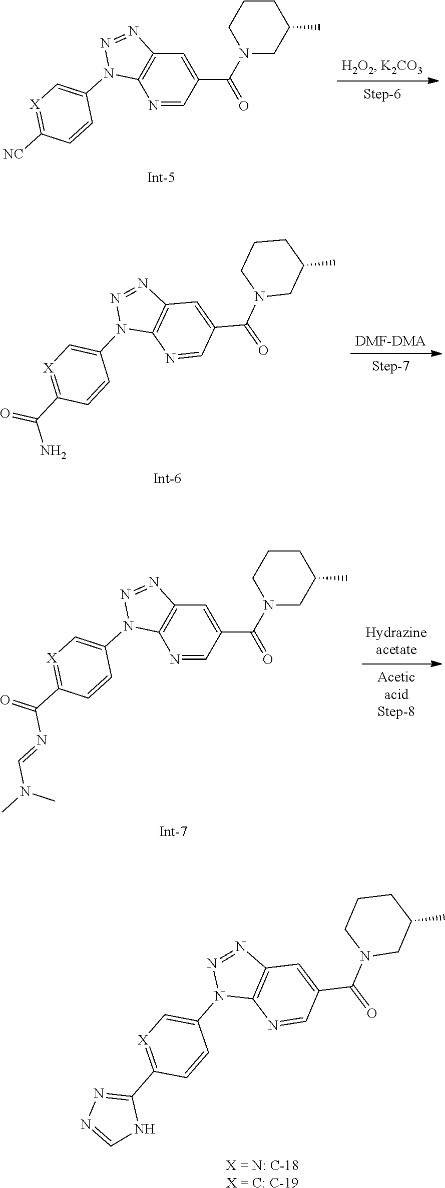

Step-1: Synthesis of methyl 6-((6-cyanopyridin-3-yl)amino)-5-nitronicotinate/methyl 6-((4-cyanophenyl)amino)-5-nitronicotinate (Int-1): To a stirred solution of methyl 6-chloro-5-nitronicotinate SM-1 (1.0 eq) in DMF (5 v) at 0° C., was added NaH (60% in paraffin oil, 2.0 eq). After 1 h, at 0° C., respective amine (SM-2) in DMF (3 v) was added and heated to 60° C. for 16 h. The progress of the reaction was monitored by TLC and LCMS. Reaction was quenched with saturated ammonium chloride and extracted with EtOAc. Combined extracts were washed with water and brine solution. Dried over sodium sulfate, filtered and concentrated under reduced pressure to afford methyl 6-((6-cyanopyridin-3-yl)amino)-5-nitronicotinate/methyl 6-((4-cyanophenyl)amino)-5-nitronicotinate Int-1a, X=N (79% yield, MS: m/z=300.05 [M+1]⁺); Int-1b, X=C (76% yield, MS: m/z=299.2 [M+1]⁺).

Step-2: Synthesis of methyl 5-amino-6-((6-cyanopyridin-3-yl)amino)nicotinate/methyl 5-amino-6-((4-cyanophenyl)amino)nicotinate (Int-2a/Int-2b): methyl 6-((6-cyanopyridin-3-yl)amino)-5-nitronicotinate/methyl 6-((4-cyanophenyl)amino)-5-nitronicotinate Int-1a/1b (1.0 eq) was reduced into methyl 5-amino-6-((6-cyanopyridin-3-yl)amino)nicotinate/methyl 5-amino-6-((4-cyanophenyl)amino)nicotinate, Int-2a/Int-2b using general reduction condition with Fe/NH₄Cl to afford Int-2a/Int-2b, X=N (60% yield, MS: m/z=270.1 [M+1]⁺)/X=C (56% yield, MS: m/z=269.3 [M+1]⁺).

Step-3: Synthesis of Int-3a/Int-3b: Using the general procedure for triazole formation with sodium nitrate, Int-2a/Int-2b was converted to Int-3a/Int-3b; X=N (88% yield, MS: m/z=280.1 [M+1]⁺); X=C (96% yield, MS: m/z=279.1 [M+1]⁺).

Step-4: Synthesis of Int-4a/Int-4b: Int-4a/Int-4b was synthesized from Int-3a/Int-3b (1.0 eq) using the general hydrolysis condition with LiOH to afford Int-4a/Int-4b; X=N (95% yield, MS: m/z=264.1 [M−1]); X=C (94% yield, MS: m/z=266.2 [M+1]⁺).

Step-5: Synthesis of Int-5a/Int-5b: Int-5a/Int-5b were synthesized from Int-4a/Int-4b (1.0 eq) and S—3-methyl piperidine (1.1 eq) using the general procedure for acid-amine coupling using HATU (1.2 eq), DIPEA (2.0 eq) in DMF (10 v) to obtain Int-5a/Int-5b; X=N (43% yield, MS: m/z=348.1 [M+1]⁺); X=C (58% yield, MS: m/z=347.1 [M+1]⁺).

Step-6: Synthesis of Int-6a/Int-6b: Int-6a/Int-6b was synthesized from Int-5a/Int-5b using general procedure for oxidation of nitrile using K₂CO₃ (2.0 eq) and H₂O₂ (5.0 eq) in DMSO (10 v) to obtain Int-6a/Int-6b as an off-white solids. X=N (58% yield, MS: m/z=366.1 [M+1]⁺); X=C (66% yield, MS: m/z=365.3 [M+1]⁺).

Step-7: Synthesis of Int-7a/Int-7b: Int-6a/Int-6b (1.0 eq) was taken in DMF-DMA (10 v) and heated to 90° C. for 1 h. The progress of the reaction was monitored with TLC. The solvent was evaporated under reduced pressure and triturated with ether to afford, Int-7a/Int-7b as off-white solids. X=N (58% yield). X=C (62% yield).

Step-8: Synthesis of (S)-(3-(4-(4H-1,2,4-triazol-3-yl)phenyl)-3H-[1,2,3]triazolo[4,5-b]pyridin-6-yl)(3-methylpiperidin-1-yl)methanone (C-18)/(S)-(3-(6-(4H-1,2,4-triazol-3-yl)pyridin-3-yl)-3H-[1,2,3]triazolo[4,5-b]pyridin-6-yl)(3-methylpiperidin-1-yl)methanone (C-19) (General procedure for triazole synthesis using hydrazine acetate): To a stirred solution of Int-7a/Int-7b (1.0 eq) in acetic acid (10 v), was added hydrazine acetate (5.0 eq) and heated to 80° C., for 1 h. The progress of the reaction was monitored with TLC LCMS. The acetic acid was evaporated, diluted with EtOAc and washed with NaHCO₃ solution, water and brine solution. The combined extracts were dried over sodium sulfate, filtered and concentrated to afford C-18/C-19 as an off-white solids, X=N, C-18 (81% yield, MS: m/z=390.0 [M+1]⁺). X=C, C-19 (76% yield, MS: m/z=389.2 [M+1]⁺).

### Example 10. Synthesis of Compounds C-4, C-6, C-12, and C-13

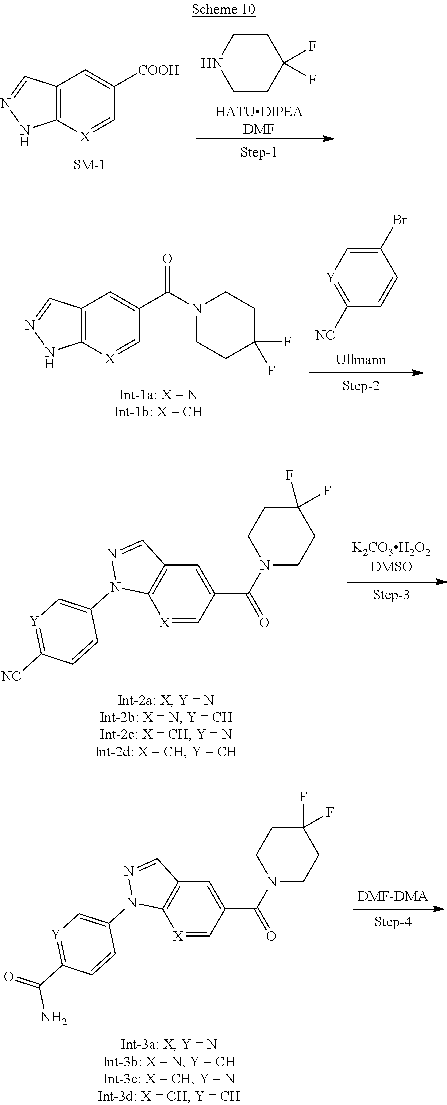

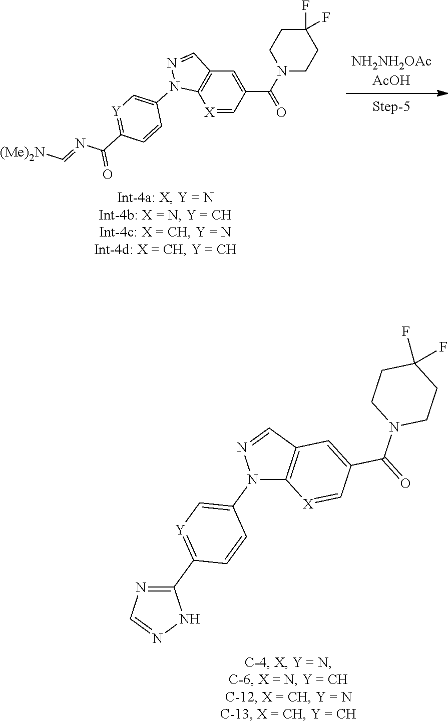

Step-1: Int1a/Int-b were prepared using the general procedure for amide coupling with HATU. The crude was purified by combi-flash column purification using 70% EtOAc/heptane to afford, Int-1a (m/z=267.1, [M+H]⁺) and Int-1b (m/z=266.2, [M+H]⁺).

Step-2: General procedure for Ullmann coupling (Int2a-d): To a stirred solution of Int-2 (1 eq) in Dioxane (100 mL), heteroaryl bromide (1.2 eq), K₃PO₄ (2.0 eq), CuI (0.2 eq), trans-dimethylcyclohexane-1,2-diamine (0.2 eq) were added at room temperature. Reaction mixture was purged with argon gas for 15 min and then continued the reaction at 100° C. for 16 h. The reaction was monitored by TLC and after completion of the reaction, quenched with sat·NH₄Cl solution (10 mL), and stirred at room temperature for 1 h. The solvent was evaporated under reduced pressure and diluted with ethyl acetate (10 mL), washed with sat. NaHCO₃ solution (50 mL), and brine solution (50 mL) and the organic phase dried over sodium sulfate, filtered and concentrated under reduced pressure to afford crude. The crude products were further purified by flash chromatography to afford Int-2a (46.4%, m/z=369.2 [M+H]+), Int-2b (51.7%, m/z=368.2, [M+H]⁺), Int-2c (43.50%, m/z=368.05, [M+H]⁺), Int-2d (58%, m/z=367.26, [M+H]⁺).

Step-3: General procedure for oxidation of nitriles (Int-3a-d): To a stirred solution (Int-2a-d) (1 eq) in DMSO (10 V) was added H₂O₂ (30%, 2.0 eq) and potassium carbonate (1.0 eq), the resultant mixture was stirred at 60° C. for 2 h. The progress of the reaction was monitored by TLC. After completion of the reaction, solid obtained was filtered, washed with Et₂O and dried in vacuo to afford Int-3a (m/z=387.2 [M+H]⁺), Int-3b (48%, m/z=386.1, [M+H]⁺), (79%, m/z=386.1, [M+H]⁺), Int-3d (86%, m/z=385.30, [M+H]⁺).

Step-4: General procedure for synthesis of Int-4a-4d using DMF-DMA: A solution of amide (Int-3a-3d) (1.0 eq) and N,N-dimethylformamide dimethyl acetal (DMF-DMA) (10 V) were stirred at 100° C. under nitrogen atmosphere for 1 h. The progress of the reaction was monitored by TLC. The mixture was evaporated under reduced pressure, solid obtained was triturated with n-pentane, filtered and dried in vacuo to afford Int-4a, Int-4b, Int-4c, and Int-4d.

Step-5A: General procedure for 1H-1, 2, 4-triazole formation using hydrazine acetate, C-4, C-6, C-12, C-13: To a stirred solution of appropriate Int-4 (1.0 eq) dissolved in acetic acid (0.5 V) was added hydrazine acetate (5.0 eq) at room temperature. The resultant mixture was stirred at 95° C. for 2 h. The progress of the reaction was monitored with TLC. After completion of the reaction, the mixture was quenched with saturated NaHCO₃ (10 mL). The solution was extracted with EtOAc (2×20 mL) and washed with water (5 mL) and brine (5 mL). The organic layer was dried over Na₂SO₄, filtered and concentrated. C-4, crude was purified by Prep-HPLC to afford C-4 (34%, m/z=411.1, [M+H]⁺) as off-white solid. C-6, the crude was purified by combi-flash column chromatography using 5% MeOH:DCM to afford C-6(54%, m/z=410.15, [M+H]⁺) as off white solid. C-12, the crude was purified by combi-flash column chromatography using 5% MeOH:DCM to afford C-12(63%, m/z=410.1, [M+H]⁺) as off white solid. C-13, the crude was purified by combi-flash column chromatography using 5% MeOH:DCM to afford C-13 (51%, m/z=409.05, [M+H]⁺) as off white solid.

### Example 11. Synthesis of Compounds C-20, C-21, and C-22

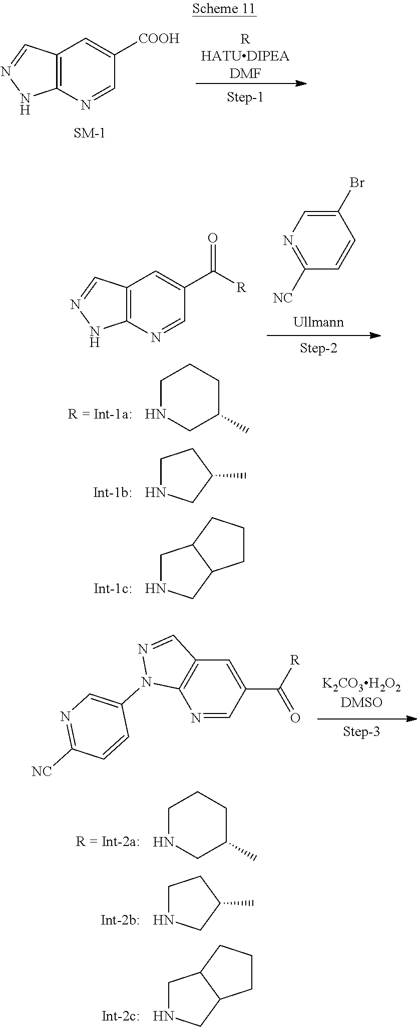

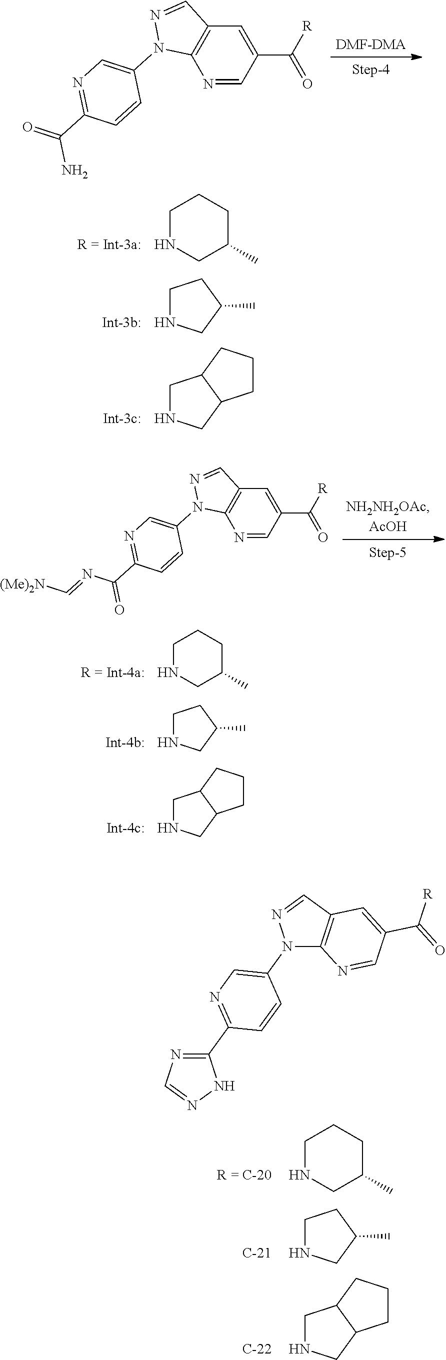

Step-1: Int1a/Int-1b/Int-1c: These were prepared using the general procedure for amide coupling with HATU. The crude was purified by combi-flash column purification using 70% EtOAc/heptane to afford, Int-1a (66% yield, m/z=245.3, [M+H]⁺) Int-1b (m/z=231.1, [M+H]⁺) and Int-1c (64.1%, m/z=257.2, [M+H]⁺).

Step-2: Int-2a/Int-2b/Int-2c: These were prepared from Int-1 using the general procedure for Ullman coupling. The crude products were further purified by flash chromatography to afford Int-2a (57.6%, m/z=347.2 [M+H]⁺), Int-2b (51.7%, m/z=332.1, [M+H]⁺), and Int-2c (35.7%, m/z=359.05, [M+H]⁺).

Step-3: Int-3a/Int-3b/Int-3c: Int-2 was converted to Int-3 using the general procedure for oxidation of nitrile with H₂O₂. After completion of the reaction, solid obtained was filtered, washed with Et₂O and dried in vacuo to afford Int-3a (63%, m/z=365.3 [M+H]⁺), Int-3b (48%, m/z=349.1, [M+H]⁺), and Int-3c (81%, m/z=377.0, [M+H]⁺).

Step-4: General procedure for synthesis of Int-4a/Int-4b/Int-4c) using DMF-DMA: A solution of amide Int-3 (1.0 eq) and N,N-dimethylformamide dimethyl acetal (DMF-DMA) (10 V) were stirred at 100° C. under nitrogen atmosphere for 1 h. The progress of the reaction was monitored by TLC. The mixture was evaporated under reduced pressure, solid obtained was triturated with n-pentane, filtered and dried in vacuo to afford Int-4a (78.9%, m/z=420.1 [M+H]⁺), Int-4b (m/z=405.1, [M+H]⁺), and Int-4c (81.9%, m/z=432.1, [M+H]⁺).

Step-5: General procedure for 1H-1, 2, 4-triazole formation using hydrazine acetate, C-20, C-21, and C-22: To a stirred solution of appropriate Int-4 (1.0 eq) dissolved in acetic acid (0.5 V) was added hydrazine acetate (5.0 eq) at room temperature. The resultant mixture was stirred at 95° C. for 2 h. The progress of the reaction was monitored with TLC. After completion of the reaction, the mixture was quenched with saturated NaHCO₃ (10 mL). The solution was extracted with EtOAc (2×20 mL) and washed with water (5 mL) and brine (5 mL). The organic layer was dried over Na₂SO₄, C-20 (59%, m/z=389.2 [M+H]⁺) as off white solid. C-21 (64%, m/z=375.3.2 [M+H]⁺), as off white solid the crude was purified by combi-flash column chromatography using 5% MeOH:DCM to afford C-22 (64%, m/z=401.1 [M+H]⁺) as off white solid.

### Example 12. Synthesis of (1-(3-(4H-1,2,4-triazol-3-yl)phenyl)-1H-pyrazolo[3,4-b]pyridin-5-yl)(4,4-difluoropiperidin-1-yl)methanone (C-41)

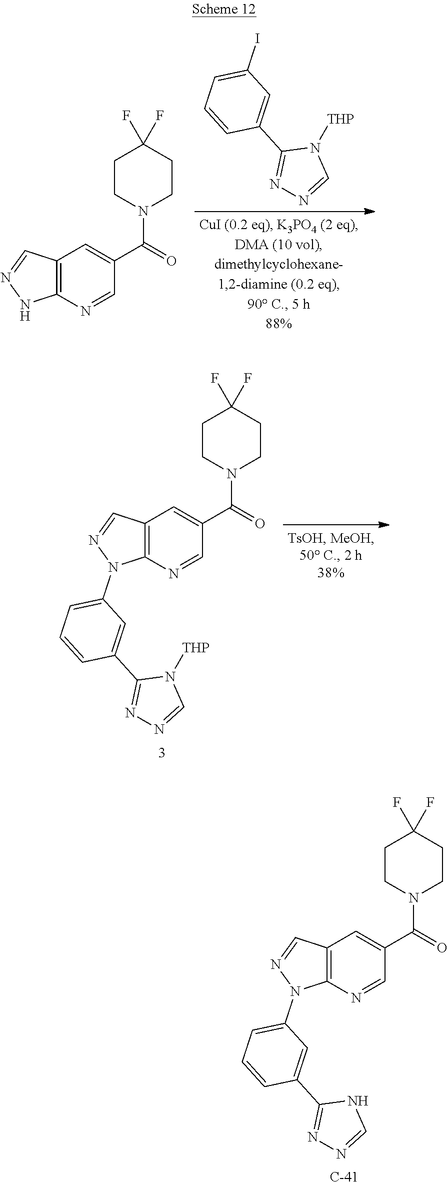

Step 1: To a mixture of (4,4-difluoropiperidin-1-yl)(1H-pyrazolo[3,4-b]pyridin-5-yl)methanone (150 mg, 563 μmol, 1.00 eq.), 3-(3-iodophenyl)-4-(tetrahydro-2H-pyran-2-yl)-4H-1,2,4-triazole (240 mg, 676 μmol, 1.20 eq.), CuI (21.5 mg, 113 μmol, 0.2 eq.), K₃PO₄ (239 mg, 1.13 mmol, 2.00 eq.), dimethylcyclohexane-1,2-diamine (16.0 mg, 113 μmol, 0.20 eq.) in DMA (1.5 mL), and the mixture was degassed and purged with N₂ 3 times, then the mixture was stirred at 90° C. for 5 hours under N₂ atmosphere (15 psi). The reaction mixture was diluted with H₂O (20 mL) and EtOAc (10 mL), then filtered to give a filtrate and extracted with EtOAc (15 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=2/1 to 1/3) to give (4,4-difluoropiperidin-1-yl)(1-(3-(4-(tetrahydro-2H-pyran-2-yl)-4H-1,2,4-triazol-3-yl)phenyl)-1H-pyrazolo[3,4-b]pyridin-5-yl)methanone (270 mg, 498 μmol, 88% yield, 91% purity) as a yellow oil. ¹H NMR (400 MHz, DMSO-d₆) δ=8.94 (s, 1H), 8.87-8.77 (m, 2H), 8.62-8.51 (m, 2H), 8.44-8.34 (m, 1H), 8.01 (d, J=7.6 Hz, 1H), 7.70 (t, J=8.0 Hz, 1H), 5.64 (dd, J=2.4, 9.6 Hz, 1H), 3.97 (br s, 1H), 3.88-3.45 (m, 5H), 2.30-1.99 (m, 7H), 1.71 (dt, J=4.0, 8.0 Hz, 1H), 1.63-1.51 (m, 2H).

Step 2: To a mixture of (4,4-difluoropiperidin-1-yl)(1-(3-(4-(tetrahydro-2H-pyran-2-yl)-4H-1,2,4-triazol-3-yl)phenyl)-1H-pyrazolo[3,4-b]pyridin-5-yl)methanone (200 mg, 405 μmol, 1.00 eq.) in MeOH (2 mL) was added p-TsOH (83.7 mg, 486 μmol, 1.20 eq.) and the mixture was stirred at 50° C. for 2 hours. The reaction mixture was diluted with H₂O (20 mL) and quenched with NaHCO₃ at 0° C. to adjust pH to neutral, and then extracted with EtOAc (20 mL×3). The combined organic layers were washed with brine (20 mL×2), dried over Na₂SO₄, filtered and concentrated under reduced pressure to give a residue. The residue was purified by prep-HPLC (column: Phenomenex luna C18 150×25 mm×10 um; mobile phase: [water (FA)−ACN]; B %: 27%-57%, 11 min) to give (1-(3-(4H-1,2,4-triazol-3-yl)phenyl)-1H-pyrazolo[3,4-b]pyridin-5-yl)(4,4-difluoropiperidin-1-yl)methanone (64.2 mg, 156 μmol, 38% yield, 99% purity) as a white solid. LCMS (ESI, M+1): m/z=410.1. ¹H NMR (400 MHz, DMSO-d₆) δ=14.23 (br s, 1H), 8.97 (t, J=2.0 Hz, 1H), 8.81 (d, J=2.0 Hz, 1H), 8.59 (s, 1H), 8.54 (d, J=2.0 Hz, 2H), 8.40-8.33 (m, 1H), 8.18-7.85 (m, 1H), 7.70 (t, J=8.0 Hz, 1H), 3.66 (br s, 4H), 2.21-1.99 (m, 4H).

### Example 13. Synthesis of [(2R)-2-methylmorpholin-4-yl]-[1-[3-(4H-1,2,4-triazol-3-yl)phenyl]pyrazolo[3,4-b]pyridin-5-yl]methanone (C-39)

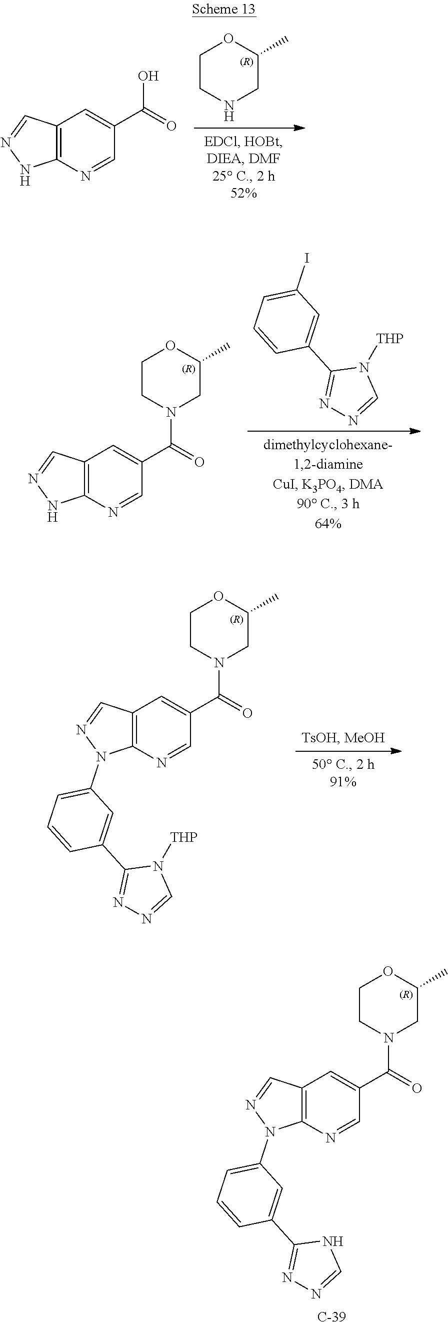

Step 1: To a mixture of 1H-pyrazolo[3,4-b]pyridine-5-carboxylic acid (4.00 g, 24.5 mmol, 1.00 eq.), (2R)-2-methylmorpholine (4.05 g, 29.4 mmol, 1.20 eq. HCl), EDCI (9.40 g, 49.0 mmol, 2.00 eq.), HOBt (6.63 g, 49.0 mmol, 2.00 eq.) in DMF (40.0 mL) was added DIEA (9.51 g, 73.6 mmol, 12.8 mL, 3.00 eq.) and the mixture was stirred at 25° C. for 2 hours. The reaction mixture was diluted with H₂O (300 mL) and extracted with EtOAc (200 mL×3). The combined organic layers were washed with brine (300 mL×2), dried over Na₂SO₄, filtered and concentrated under reduced pressure to give a residue. The residue was purified by reversed-phase HPLC (basic condition). The desired fraction was collected and lyophilized to give [(2R)-2-methylmorpholin-4-yl]-(1H-pyrazolo[3,4-b]pyridin-5-yl)methanone (3.20 g, 12.7 mmol, 52% yield, 98% purity) as a yellow solid. ¹H NMR (400 MHz, DMSO-d6) δ=13.87 (br s, 1H), 8.57 (d, J=2.0 Hz, 1H), 8.33 (d, J=2.0 Hz, 1H), 8.23 (s, 1H), 4.57-4.04 (m, 1H), 3.94-3.73 (m, 1H), 3.69-3.40 (m, 3H), 3.21-2.70 (m, 2H), 1.26-0.95 (m, 3H).

Step 2: To a mixture of [(2R)-2-methylmorpholin-4-yl]-(1H-pyrazolo[3,4-b]pyridin-5-yl)methanone (200 mg, 812 μmol, 1.00 eq.), 3-(3-iodophenyl)-4-tetrahydropyran-2-yl-1,2,4-triazole (346 mg, 975 μmol, 1.20 eq.), CuI (30.9 mg, 162 μmol, 0.20 eq.), K₃PO₄ (345 mg, 1.62 mmol, 2.00 eq.) and (1R,2R)—N1,N2-dimethylcyclohexane-1,2-diamine (23.1 mg, 162 μmol, 0.20 eq.) in DMA (4.00 mL) was degassed and purged with N₂ for 3 times, and then the mixture was stirred at 90° C. for 3 hours under N₂ atmosphere (15 psi). The reaction mixture was filtered and diluted with H₂O (20.0 mL), then extracted with EtOAc (20.0 mL×3). The combined organic layers were washed with brine (20.0 mL×1), dried over Na₂SO₄, filtered and concentrated under reduced pressure to give a residue. The residue was purified by column chromatography on silica gel (petroleum ether/ethyl acetate=3/1 to 0/1) to give [(2R)-2-methylmorpholin-4-yl]-[1-[3-(4-tetrahydropyran-2-yl-1,2,4-triazol-3-yl)phenyl]pyrazolo[3,4-b]pyridin-5-yl]methanone (254 mg, 520 μmol, 64% yield, 97% purity) as a white solid. LCMS (ESI, M-84): m/z=390.0.

Step 3: To a mixture of [(2R)-2-methylmorpholin-4-yl]-[1-[3-(4-tetrahydropyran-2-yl-1,2,4-triazol-3-yl)phenyl]pyrazolo[3,4-b]pyridin-5-yl]methanone (100 mg, 211 μmol, 1.00 eq.) in MeOH (2.00 mL) was added TsOH (43.6 mg, 253 μmol, 1.20 eq.). Then the mixture was stirred at 50° C. for 2 hours. The reaction mixture was diluted with saturated NaHCO₃ (10.0 mL) and extracted with EtOAc (10.0 mL×3). The combined organic layers were washed with brine (10.0 mL×1), dried over Na₂SO₄, filtered and concentrated under reduced pressure to give a residue. The crude product was purified by prep-HPLC (column: Unisil 3-100 C18 Ultra 150×50 mm×3 um; mobile phase: [water (FA)−ACN]; B %: 22%-52%, 7 minutes) to give [(2R)-2-methylmorpholin-4-yl]-[1-[3-(4H-1,2,4-triazol-3-yl)phenyl]pyrazolo[3,4-b]pyridin-5-yl]methanone (75.7 mg, 192 μmol, 91% yield, 99% purity) as a white solid. LCMS (ESI,M+1): m/z=390.0. ¹H NMR (400 MHz, DMSO-d₆) δ=14.35-14.26 (m, 1H), 8.98 (s, 1H), 8.78 (d, J=2.0 Hz, 1H), 8.68-8.46 (m, 3H), 8.38 (br d, J=8.4 Hz, 1H), 8.03 (d, J=7.6 Hz, 1H), 7.69 (t, J=8.0 Hz, 1H), 4.38-4.35 (m, 1H), 3.88-3.77 (m, 1H), 3.66-3.45 (m, 3H), 3.33-3.13 (m, 1H), 2.99-2.89 (m, 1H), 1.15-1.04 (m, 3H).

### Example 14. Synthesis of (1-(3-chloro-5-(4H-1,2,4-triazol-3-yl)phenyl)-1H-pyrazolo[3,4-b]pyridin-5-yl)(thiomorpholino)methanone (C-66)

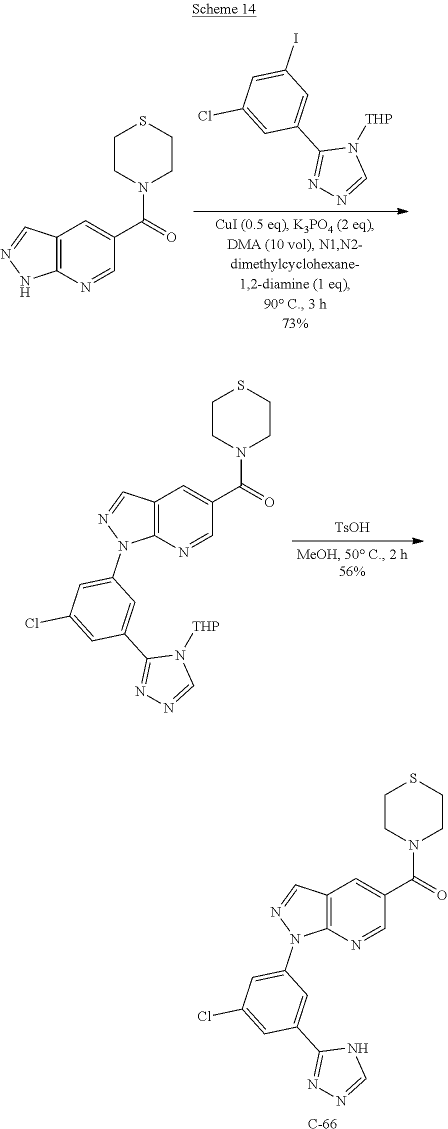

Step 1: A mixture of (1H-pyrazolo[3,4-b]pyridin-5-yl)(thiomorpholino)methanone (150 mg, 604 μmol, 1.00 eq.), 3-(3-chloro-5-iodophenyl)-4-(tetrahydro-2H-pyran-2-yl)-4H-1,2,4-triazole (282 mg, 725 μmol, 1.20 eq.), (1R,2R)—N1,N2-dimethylcyclohexane-1,2-diamine (85.9 mg, 604 μmol, 1.00 eq.), K₃PO₄ (256 mg, 1.21 mmol, 2.00 eq.) and CuI (57.5 mg, 302 μmol, 0.50 eq.) in DMAC (1 mL) was degassed and purged with N₂ for 3 times, and then the mixture was stirred at 90° C. for 3 hours under N₂ atmosphere (15 psi). The mixture was diluted with H₂O (30 mL) and extracted with EA (20 mL×3), the organic layers were washed with saturated salt solution (30 mL×3), dried over Na₂SO₄, filtered and concentrated under reduced pressure to give a reside. The residue was purified by column chromatography (SiO₂, Petroleum ether/Ethyl acetate=3/1 to 1/1) to give (1-(3-chloro-5-(4-(tetrahydro-2H-pyran-2-yl)-4H-1,2,4-triazol-3-yl)phenyl)-1H-pyrazolo[3,4-b]pyridin-5-yl)(thiomorpholino)methanone (250 mg, 441 μmol, 73% yield, 90% purity) as a yellow liquid. LCMS (ESI, M+1): m/z=510.2. ¹H NMR (400 MHz, DMSO-d₆) δ=8.98-8.92 (m, 1H), 8.90 (s, 1H), 8.82 (d, J=2.0 Hz, 1H), 8.62 (s, 1H), 8.57 (t, J=2.0 Hz, 1H), 8.52 (d, J=2.0 Hz, 1H), 8.03-7.91 (m, 1H), 5.66 (dd, J=2.4, 9.6 Hz, 1H), 3.97-3.58 (m, 5H), 3.31 (s, 2H), 2.77-2.66 (m, 4H), 2.24-2.13 (m, 1H), 2.10-2.03 (m, 1H), 1.77-1.56 (m, 3H).

Step 2: To a solution of (1-(3-chloro-5-(4-(tetrahydro-2H-pyran-2-yl)-4H-1,2,4-triazol-3-yl)phenyl)-1H-pyrazolo[3,4-b]pyridin-5-yl)(thiomorpholino)methanone (200 mg, 392 μmol, 1.00 eq.) in MeOH (2 mL) was added TsOH (81.0 mg, 471 μmol, 1.20 eq.). The mixture was stirred at 50° C. for 2 hours. The reaction mixture was extracted with EtOAc (30 mL), then added saturated sodium bicarbonate to added pH=8 and diluted with H₂O (40 mL). The combined organic layers were washed with brine (40 mL×2), dried over Na₂SO₄, filtered and concentrated under reduced pressure to give a residue. The residue was purified by prep-HPLC (column: Phenomenex luna C18 150×25 mm×10 um; mobile phase: [water (FA)−ACN]; B %: 40%-70%, 10 min) to give (1-(3-chloro-5-(4H-1,2,4-triazol-3-yl)phenyl)-1H-pyrazolo[3,4-b]pyridin-5-yl)(thiomorpholino)methanone (95.0 mg, 220 μmol, 56% yield, 98.5% purity) as a white solid. LCMS (ESI, M+1): m/z=426.0. ¹H NMR (400 MHz, DMSO-d₆) δ=14.42 (br dd, J=3.2, 5.6 Hz, 1H), 9.04-8.96 (m, 1H), 8.82 (d, J=2.0 Hz, 1H), 8.75-8.59 (m, 2H), 8.57-8.47 (m, 2H), 8.08-7.92 (m, 1H), 4.03-3.59 (m, 4H), 2.69 (br d, J=12.0 Hz, 4H).

The additional compounds of this application have been synthesized by the methods described in Examples 1-14.

Analytical data for compounds described herein can be seen in Table 2a.

TABLE 2a
Analytica data for representative compounds described herein.

| | | Mass Spec. | |
|---|---|---|---|
| | | Calculated/ | |
| Compound | Yield/ | Mass Spec. | |
| No | Purity | Found (m/z) | ¹H NMR (DMSO-d₆, 400 MHz) |
| C-1 | 45.28%/96.32% | 387.11 for | δ 13.32-13.17 (m, 1H), 8.98 (d, J = 1.9 Hz, 1H), |
| | | C₁₈H₁₅F₂N₅O₃/ | 8.89 (d, J = 1.8 Hz, 1H), 8.45 (d, J = 8.8 Hz, 2H), |
| | | 388.1 | 8.26 (d, J = 8.8 Hz, 2H), 3.91-3.70 (m, 2H), 3.62- |
| | | (M + 1) | 3.40 (m, 2H), 2.20-2.03 (m, 4H). |
| C-2 | 31%/99.06% | 386.12 for | δ 13.08-12.95 (m, 1H), 8.83-8.80 (m, 1H), 8.62 |
| | | C₁₉H₁₆F₂N₄O₃/ | (s, 1H), 8.55 (d, J = 2.0 Hz, 1H), 8.50 (d, J = 8.8 |
| | | 385.25 | Hz, 2H), 8.16 (d, J = 8.8 Hz, 2H), 3.92-3.50 (m, |
| | | (M − 1) | 4H), 2.16-2.04 (m, 4H). |
| C-3 | 81%/99.20% | 400.13 for | δ 13.35-13.12 (m, 1H), 8.35 (d, J = 1.9 Hz, 1H), |
| | | C₂₀H₁₈F₂N₄O₃/ | 8.20-8.14 (m, 3H), 7.75 (d, J = 8.6 Hz, 2H), 3.84- |
| | | 401.1 | 3.49 (m, 4H), 2.56-2.54 (s, 3H), 2.13-2.02 (m, |
| | | (M + 1) | 4H). |
| C-4 | 34%/94.82% | 410.14 for | δ 14.92-14.16 (m, 1H), 9.64-9.54 (m, 1H), 8.95- |
| | | C₁₉H₁₆F₂N₈O/ | 8.73 (m, 2H), 8.73-8.63 (m, 1H), 8.58 (s, 1H), |
| | | 411.2 | 8.33 (br d, J = 8.5 Hz, 1H), 8.15-8.07 (m, 1H), |
| | | (M + 1) | 3.87-3.46 (m, 4H), 2.10 (br s, 4H). |
| C-5 | 74.71%/ | 411.14 for | δ 15.06-14.31 (m, 1H), 9.54 (br s, 1H), 9.00 (d, J = |
| | 95.61% | C₁₈H1₅F₂N₉O/ | 1.9 Hz, 1H), 8.93-8.91 (m, 1H), 8.87-8.76 (m, |
| | | 412.1 | 1H), 8.42 (d, J = 8.5 Hz, 1H), 8.28-8.01 (m, 1H), |
| | | (M + 1) | 3.86-3.47 (m, 4H), 2.17-2.05 (m, 4H). |
| C-6 | 54%/97.29% | 409.15 for | δ 14.25-14.04 (m, 1H), 8.81 (d, J = 2.0 Hz, 1H), |
| | | C₂₀H₁₇F₂N₇O/ | 8.69-8.52 (m, 3H), 8.48-8.38 (m, 2H), 8.25- |
| | | 410.15 | 8.20 (m, 2H), 3.82-3.44 (m, 4H), 2.16-2.03 (m, |
| | | (M + 1) | 4H). |
| C-7 | 67.39%/ | 410.14 for | δ 14.28-14.19 (m, 1H), 8.97 (d, J = 1.9 Hz, 1H), |
| | 95.09% | C₁₉H₁₆F₂N₈O/ | 8.88 (d, J = 1.6 Hz, 1H), 8.71-8.68 (m, 1H), 8.34 |
| | | 411.1 | (br s, 4H), 3.85-3.68 (m, 2H), 3.62-3.43 (m, |
| | | (M + 1) | 2H), 2.17-2.06 (m, 4H). |
| C-8 | 9.70%/ | 425.15 for | δ 9.52-9.49 (m, 1H), 8.99 (d, J = 1.8 Hz, 1H), |
| | 95.08% | C₁₉H₁₇F₂N₉O/ | 8.91 (d, J = 1.8 Hz, 1H), 8.76 (dd, J = 8.6, 2.6 Hz, |
| | | 426.2 | 1H), 8.65 (s, 1H), 8.39-8.35 (m, 1H), 4.01-3.99 |
| | | (M + 1) | (m, 3H), 3.86-3.50 (m, 4H), 2.18-2.08 (m, 4H). |
| C-9 | %/96.80% | 410.14 for | δ 15.09-14.31 (m, 1H), 9.28 (br s, 1H), 8.79- |
| | | C₁₉H₁₆F₂N₈O/ | 8.54 (m, 1H), 8.51-8.37 (m, 1H), 8.35-8.29 (m, |
| | | 411.5 | 1H), 8.20-8.13 (m, 2H), 7.62-7.57 (m, 1H), 3.82- |
| | | (M + 1) | 3.70 (m, 2H), 3.48-3.37 (m, 2H), 2.16-2.00 (m, |
| | | | 4H). |
| C-10 | 54%/99.84% | 409.15 for | δ 14.29-14.21 (m, 1H), 8.71 (s, 1H), 8.36-8.25 |
| | | C₂₀H₁₇F₂N₇O/ | (m, 3H), 8.14-8.00 (m, 3H), 7.58 (d, J = 8.5 Hz, |
| | | 410.05 | 1H), 3.84-3.36 (m, 4H), 2.18-1.93 (m, 4H). |
| | | (M + 1) | |
| C-11 | 24.5%/95.44% | 386.12 for | δ 13.38-13.19 (m, 1H), 8.35 (s, 1H), 8.24 (d, J = |
| | | C₁₉H₁₆F₂N₄O₃/ | 8.6 Hz, 2H), 8.13-8.06 (m, 3H), 7.77 (dd, J = 8.6, |
| | | 387.0 | 1.3 Hz, 1H), 3.84-3.40 (m, 4H), 2.18-2.00 (m, |
| | | (M + 1) | 4H). |
| C-12 | 63%/85.71% | 409.15 for | δ 9.17 (br s, 1H), 8.60-8.57 (m, 1H), 8.44 (br d, |
| | | C₂₀H₁₇F₂N₇O/ | J = 8.3 Hz, 1H), 8.31 (br d, J = 8.3 Hz, 1H), 8.11- |
| | | 410.2 | 8.03 (m, 2H), 7.68-7.62 (m, 1H), 3.79-3.65 (m, |
| | | (M + 1) | 4H), 2.08 (br s, 4H). |
| C-13 | 51%/99.38% | 408.15 for | δ 14.28-14.10 (m, 1H), 8.76-8.57 (m, 1H), 8.51 |
| | | C₂₁H₁₈F₂N₆O/ | (s, 1H), 8.24 (br d, J = 8.5 Hz, 2H), 8.06 (s, 1H), |
| | | 407.35 | 8.02-7.89 (m, 3H), 7.61 (dd, J = 8.7, 1.4 Hz, 1H), |
| | | (M + 1) | 3.75-3.49 (m, 4H), 2.13-2.02 (m, 4H). |
| C-14 | 25.5%/99.57% | 385.12 for | δ 13.12-13.05 (m, 1H), 8.54 (d, J = 0.8 Hz, 1H), |
| | | C₂₀H₁₇F₂N₃O₃/ | 8.19-8.12 (m, 2H), 8.09-8.01 (m, 2H), 8.00- |
| | | 386.2 | 7.94 (m, 2H), 7.63 (dd, J = 8.8, 1.6 Hz, 1H), 3.73- |
| | | (M + 1) | 3.48 (m, 4H), 2.13-2.01 (m, 4H). |
| C-15 | 7.3%/95.02% | 409.15 for | δ 9.08 (d, J = 1.9 Hz, 1H), 8.82 (s, 1H), 8.43-8.25 |
| | | C₂₀H₁₇F₂N₇O/ | (m, 3H), 7.87 (d, J = 8.4 Hz, 1H), 7.84-7.80 (m, |
| | | 410.15 | 1H), 7.42 (dd, J = 8.3, 1.3 Hz, 1H), 3.78-3.44 (m, |
| | | (M + 1) | 4H), 2.10-1.96 (m, 4H). |
| C-16 | 37.30%/99.82% | 408.15 for | δ 14.57-13.84 (m, 1H), 8.75 (s, 1H), 8.60-8.47 |
| | | C₂₁H₁₈F₂N₆O/ | (m, 1H), 8.29-8.23 (m, 2H), 7.84 (dd, J = 8.4, 3.8 |
| | | 409.10 | Hz, 3H), 7.80-7.75 (m, 1H), 7.41 (dd, J = 8.3, 1.5 |
| | | (M + 1) | Hz, 1H), 3.76-3.47 (m, 4H), 2.14-1.95 (m, 4H). |
| C-17 | 19.28%/97.72% | 385.12 for | δ 13.21 (br s, 1H), 8.77 (s, 1H), 8.18 (d, J = 8.6 |
| | | C₂₀H₁₇F₂N₃O₃/ | Hz, 2H), 7.92-7.83 (m, 3H), 7.78 (d, J = 8.3 Hz, |
| | | 386.2 | 1H), 7.46 (dd, J = 8.4, 1.2 Hz, 1H), 3.75-3.46 (m, |
| | | (M + 1) | 4H), 2.16-1.90 (m, 4H). |
| C-18 | 81%/98.57% | 389.17 for | δ 15.09-14.12 (m, 1H), 9.54 (d, J = 2.1 Hz, 1H), |
| | | C₁₉H₁₉N₉O/ | 8.93 (d, J = 1.8 Hz, 1H), 8.84 (s, 2H), 8.42 (d, J = |
| | | 388.25 | 8.6 Hz, 2H), 4.46-4.35 (m, 1H), 3.59-3.48 (m, |
| | | (M + 1) | 1H), 3.16-4 (m, 2H), 1.85-1.78 (m, 2H), 1.72- |
| | | | 1.65 (m, 1H), 1.60-1.53 (m, 1H), 1.24-1.15 (m, |
| | | | 1H), 1.01-0.72 (m, 3H). |
| C-19 | 76%/94.39% | 388.18 for | δ 8.92-8.87 (m, 1H), 8.79-8.75 (m, 1H), 8.52 (s, |
| | | C₂₀H₂₀N₈O/389.2 | 1H), 8.40-8.30 (m, 4H), 4.38 (br d, J = 7.0 Hz, |
| | | (M + 1) | 1H), 3.65-3.50 (m, 2H), 1.85-1.74 (m, 2H), 1.71- |
| | | | 1.45 (m, 2H), 1.31-1.12 (m, 2H), 1.00-0.68 (m, |
| | | | 3H). |
| C-20 | 59%/93.46% | 388.18 for | δ 15.14-14.11 (m, 1H), 9.61-9.58 (m, 1H), 8.92- |
| | | C₂₀H₂₀N₈O/389.2 | 8.86 (m, 1H), 8.78-8.75 (m, 1H), 8.67-8.64 (m, |
| | | (M + 1) | 1H), 8.49 (d, J = 1.8 Hz, 2H), 8.32 (d, J = 8.5 Hz, |
| | | | 1H), 4.42-4.31 (m, 1H), 3.63-3.50 (m, 1H), 3.13- |
| | | | 9 (m, 2H), 1.85-1.79 (m, 1H), 1.70-1.49 (m, |
| | | | 3H), 1.24-1.16 (m, 1H), 0.99-0.75 (m, 3H). |
| C-21 | 64%/96.33% | 374.16 for | δ 14.98-14.05 (m, 1H), 9.63-9.54 (m, 1H), 8.95- |
| | | C₁₉H₁₈N₈O/ | 8.88 (m, 2H), 8.69-8.61 (m, 2H), 8.35-8.30 (m, |
| | | 375.35 | 1H), 8.13-8.09 (m, 1H), 3.76-3.53 (m, 3H), 3.21- |
| | | (M + 1) | 3.07 (m, 1H), 2.28-2.16 (m, 1H), 2.10-1.95 (m, |
| | | | 1H), 1.60-1.46 (m, 1H), 1.11-0.97 (m, 3H). |
| C-22 | 64%/96.50% | 400.18 for | δ 15.12-13.97 (m, 1H), 9.64-9.57 (m, 1H), 8.87 |
| | | C₂₁H₂₀N₈O/401.10 | (d, J = 2.0 Hz, 2H), 8.67-8.60 (m, 2H), 8.32 (br |
| | | (M + 1) | d, J = 8.7 Hz, 1H), 8.15-8.05 (m, 1H), 3.86-3.67 |
| | | | (m, 2H), 3.45-3.32 (m, 2H), 2-2.61 (m, 2H), |
| | | | 1.86-1.68 (m, 3H), 1.61-1.35 (m, 3H). |

### Example 15. Synthesis of 4-(7-(4,4-difluoropiperidine-1-carbonyl)imidazo[1,2-a]pyridin-3-yl)benzoic acid (D-1)/5-(7-(4,4-difluoropiperidine-1-carbonyl)imidazo[1,2-a]pyridin-3-yl)picolinic acid (D-2)

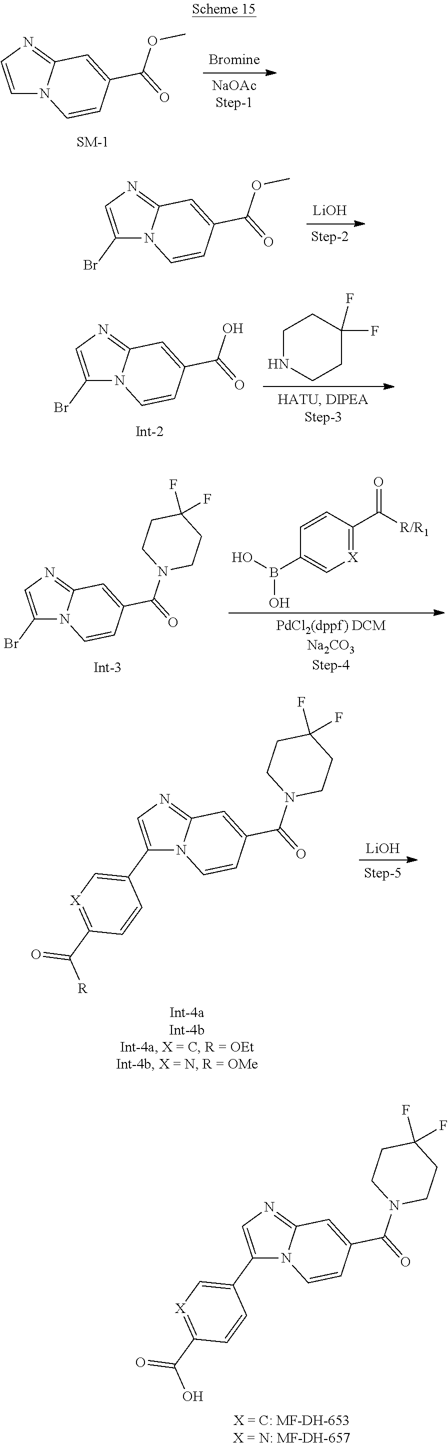

Step-1: Synthesis of methyl 3-bromoimidazo[1,2-a]pyridine-7-carboxylate (Int-2):

To a stirred solution of methyl imidazo[1,2-a]pyridine-7-carboxylate (SM-1) (1.0 g, 5.6 mmol, 1.0 eq) in methanol (10 mL) at 0° C., sodium acetate (0.56 g, 6.8 mmol, 1.2 eq) and Bromine (0.32 mL, 6.2 mmol, 1.1 eq) were added. The reaction was stirred at RT for 1 h. The progress of the reaction was monitored by TLC. The reaction was diluted with ice water, solid was filtered to obtain methyl 3-bromoimidazo[1,2-a]pyridine-7-carboxylate (Int-1) (1.3 g, 89.6% MS: m/z=257.0 [M+2]⁺ as an off-white solid.

Step-2: Synthesis of 3-bromoimidazo[1,2-a]pyridine-7-carboxylic acid (Int-2): 3-bromoimidazo[1,2-a]pyridine-7-carboxylic acid (Int-2) was synthesized from methyl 3-bromoimidazo[1,2-a]pyridine-7-carboxylate (Int-1) (1.3 g, 5.0 mmol, 1.0 eq) using the general hydrolysis condition with lithium hydroxide (0.63 g, 15.0 mmol, 3.0 eq) to obtain 3-bromoimidazo[1,2-a]pyridine-7-carboxylic acid (Int-2) (1.1 g, 90%, MS: m/z=243.0 [M+2]⁺) as an off-white solid.

Step-3: Synthesis of (3-bromoimidazo[1,2-a]pyridin-7-yl)(4,4-difluoropiperidin-1-yl)methanone (Int-3): (3-bromoimidazo[1,2-a]pyridin-7-yl)(4,4-difluoropiperidin-1-yl)methanone (Int-3) was synthesized from 3-bromoimidazo[1,2-a]pyridine-7-carboxylic acid (Int-2) (1.1 g, 4.5 mmol, 1.0 eq) using the general procedure for amide coupling with HATU to afford (3-bromoimidazo[1,2-a]pyridin-7-yl)(4,4-difluoropiperidin-1-yl)methanone (Int-3) (1.42 g, 91.61%, MS: 345.2 [M+1]⁺) as an off-white solid.

Step-4: Synthesis of Int-4: In a sealed tube, to the stirred solution of (3-bromoimidazo[1,2-a]pyridin-7-yl)(4,4-difluoropiperidin-1-yl)methanone (Int-3) (1.0 eq) in 1,4-dioxane:water (8:2, 10 v), degassed with argon. The respective boronic acid (1.3 eq) and sodium carbonate (3.0 eq) was added. After 15 min, Pd(dppf)Cl₂ DCM adduct (0.1 eq) was added and stirred at 90° C., for 8 h. The progress of the reaction was monitored by the TLC. The reaction mass was filtered through the celite bed and the organic layer was washed with water and brine solution, dried over sodium sulfate, filtered and concentrated. The crude was purified using combi flash, 60% EtOAc/Hex as an eluent. to afford obtain Int-4a (X=C and R=OEt, 75% yield, MS: m/z=414.61 [M+1]⁺) and Int-4b X=N and R=OMe, 34% yield, MS: m/z=401.2 [M+1]) as an off-white solid.

Step-5: Synthesis of 4-(7-(4,4-difluoropiperidine-1-carbonyl)imidazo[1,2-a]pyridin-3-yl)benzoic acid (D-1)/5-(7-(4,4-difluoropiperidine-1-carbonyl)imidazo[1,2-a]pyridin-3-yl)picolinic acid (D-2): 4-(7-(4,4-difluoropiperidine-1-carbonyl)imidazo[1,2-a]pyridin-3-yl)benzoic acid D-1/5-(7-(4,4-difluoropiperidine-1-carbonyl)imidazo[1,2-a]pyridin-3-yl)picolinic acid MF-DH-657 was synthesized from Int-4a/Int-4b using general procedure for hydrolysis using LiOH to afford D-1 (19.2% yield, MS: m/z=386.1 [M+1]⁺) and D-2 (62% yield, MS: m/z=387.2[M+1]⁺).

### Example 16. Synthesis of (3-(6-(1H-1,2,4-triazol-5-yl)pyridin-3-yl)imidazo[1,2-a]pyridin-7-yl)(4,4-difluoropiperidin-1-yl)methanone (D-3)

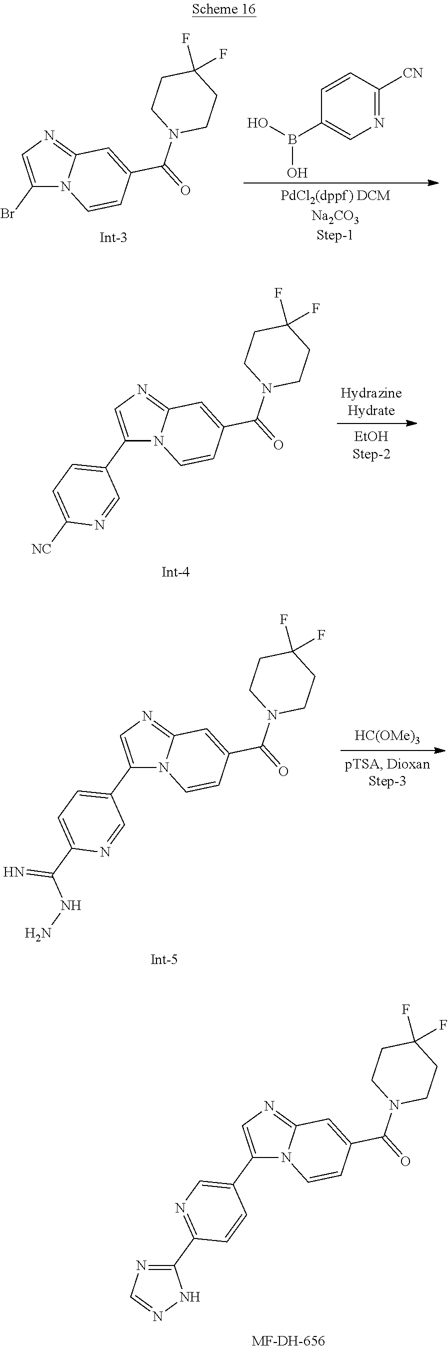

Synthesis of Int-3 is described above in the synthesis of D-1.

Step-1: Synthesis of 5-(7-(4,4-difluoropiperidine-1-carbonyl)imidazo[1,2-a]pyridin-3-yl)picolinonitrile (Int-4): In a sealed tube, to the stirred solution of (3-bromoimidazo[1,2-a]pyridin-7-yl)(4,4-difluoropiperidin-1-yl)methanone (Int-3) (0.6 g, 1.74 mmol, 1.0 eq) in 1,4-Dioxane:water (8:2, 10 v), degassed with argon. (6-cyanopyridin-3-yl)boronic acid (0.33 g, 2.2 mmol, 1.3 eq), sodium carbonate (0.55 g, 1.74 mmol, 3.0 eq) was added. After 15 min, Pd(dppf)Cl₂ DCM adduct (0.14 g, 0.174 mmol, 0.1 eq) was added and stirred at 90° C., for 8 h. The progress of the reaction was monitored by the TLC. The reaction mass was filtered through the celite bed and the organic layer was washed with water and brine solution, dried over sodium sulfate, filtered and concentrated. Crude was purified using combi flash, 60% EtOAc/Hex as an eluent to afford 5-(7-(4,4-difluoropiperidine-1-carbonyl)imidazo[1,2-a]pyridin-3-yl)picolinonitrile, Int-4 (0.39 g, 60.9% yield, MS: m/z=368.15 [M+1]⁺) as an off-white solid.

Step-2: Synthesis of 5-(7-(4,4-difluoropiperidine-1-carbonyl)imidazo[1,2-a]pyridin-3-yl)picolinimidohydrazide (Int-5): To a stirred solution of 5-(7-(4,4-difluoropiperidine-1-carbonyl)imidazo[1,2-a]pyridin-3-yl)picolinonitrile Int-4 (0.15 g, 0.42 mmol, 1.0 eq) in ethanol (10 v) Hydrazine hydrate (2 mL) was added and stirred at 80° C. for 1 h. The progress of the reaction was monitored by TLC and LCMS. The solvent was concentrated under reduced pressure, triturated with pentane to afford Int-5 (0.13 g, 79.7% m/z=400.1 [M+1]⁺) which was used in the next step without further purification.

Step-3: Synthesis of (3-(6-(1H-1,2,4-triazol-5-yl)pyridin-3-yl)imidazo[1,2-a]pyridin-7-yl)(4,4-difluoropiperidin-1-yl)methanone (D-3): To Int-5 (0.13 g, 0.32 mmol, 1.0 eq) in Dioxane (10 v) was added p-TSA (0.012 g, 0.06 mmol, 0.2 eq) and trimethyl orthoformate (0.17 g, 0.16 mmol, 5.0 eq) and heated to 90° C. for 3 h. The progress of the reaction was monitored with TLC and LCMS. The reaction mixture was diluted with EtOAC (4×10 mL), washed with 10% Na₂CO₃. Organic layer was dried over sodium sulfate, filtered and concentrated. The crude was purified using prep HPLC to afford (3-(6-(1H-1,2,4-triazol-5-yl)pyridin-3-yl)imidazo[1,2-a]pyridin-7-yl)(4,4-difluoropiperidin-1-yl)methanone, D-3 (40 mg, 30%, MS: m/z=410.2 [M+1]⁺).

Analytical data for compounds described herein can be seen in Table 2b.

TABLE 2b
Analytica data for representative compounds described herein.

| | | Mass Spec. | |
|---|---|---|---|
| Compound | Yield/ | Calculated/Mass | |
| No. | Purity | Spec. Found (m/z) | ¹H NMR (DMSO-d₆,400 MHz) |
| D-1 | 19.2%/ | 385.12 for | δ 13.11-12.83 (m, 1H), 8.75-8.70 (m, 1H), 8.13- |
| | 94.60% | C₂₀H₁₇F₂N₃O₃/386.1 | 8.08 (m, 2H), 8.02 (s, 1H), 7.89-7.80 (m, 3H), 7.09- |
| | | (M + 1) | 7.04 (m, 1H), 3.77-3.50 (m, 4H), 2.16-2.01 (m, 4H). |
| D-3 | 30%/ | 409.15 for | δ 14.88-14.38 (m, 1H), 9.02 (d, J = 1.6 Hz, 1H), 8.75 |
| | 92.80% | C₂₀H₁₇F₂N₇O/410.20 | (d, J = 7.0 Hz, 1H), 8.34 (br dd, J = 8.3, 2.1 Hz, 2H), |
| | | (M + 1) | 8.28-8.19 (m, 1H), 8.10 (s, 1H), 7.84 (s, 1H), 7.08 |
| | | | (dd, J = 7.1, 1.6 Hz, 1H), 3.77-3.48 (m, 4H), 2.09 (br |
| | | | s, 4H). |
| D-2 | 62%/ | 386.12 for | δ 8.75-8.71 (m, 1H), 8.67-8.62 (m, 1H), 8.15-8.11 |
| | 96.50% | C₁₉H₁₆F₂N₄O₃/385.20 | (m, 1H), 8.04-7.98 (m, 2H), 7.82-7.78 (m, 1H), 7.08- |
| | | (M + 1) | 7.02 (m, 1H), 3.75-3.52 (m, 4H), 2.16-2.03 (m, |
| | | | 4H). |
| D-4 | 11.5%/ | 385.12 for | δ 13.23-13.11 (m, 1H), 9.33-9.30 (m, 1H), 8.29- |
| | 99.89% | C₂₀H₁₇F₂N₃O₃/386.2 | 8.24 (m, 2H), 8.17-8.12 (m, 2H), 7.95-7.92 (m, 1H), |
| | | (M + 1) | 7.82-7.77 (m, 1H), 7.41-7.37 (m, 1H), 3.70-3.56 |
| | | | (m, 4H), 2.14-2.03 (m, 4H). |

### BIOLOGICAL EXAMPLES

### Example B-1. hPGDH Inhibitor Screening Biochemical Assay

A hydroxyprostaglandin dehydrogenase inhibition screening biochemical assay can be performed to assess the synthesized inhibitors provided herein. Provided herein is an exemplary biochemical assay for hPGDH inhibitor screening.

The in vitro biochemical assay can be performed in white, 384 plates in total 20 μl reaction volume consisting of 10 nM of 15-PGDH/HPGD (R&D System #5660-DH), 15 μM Prostaglandin E2 (Sigma, Cat #P5640-10MG) and 0.25 mM β-Nicotinamide adenine dinucleotide sodium salt (Sigma, Cat #N0632-5G) made in reaction buffer (50 mM Tris-HCl, pH 7.5, 0.01% Tween 20) at 10-point dose response curve for test/tool compounds. Briefly, 5 μl (4×) of compounds solution and 5 μl (final concentration, 10 nM) of enzyme solution is added to white 384 well plates and incubated for 10 mins at 37° C. 5 μl (4×) of Prostaglandin E2 and 5 μl (4×) of β-Nicotinamide adenine dinucleotide sodium salt is added to the wells and incubated for 10 mins at room temperature. Fluorescence is recorded at ex/em=340 nm/485 nm. The percentage (0%) inhibition of enzyme activity was determined relative to positive control (1% DMSO) and IC50 was calculated using GraphPad prism software (four parameter-variable slope equation). Exemplary data are shown in Table 3.

TABLE 3
hPGDH inhibition potency.

| Compound | hPGDH: Average | Compound | hPGDH: Average |
|---|---|---|---|
| number | IC₅₀ (μM) | number | IC₅₀ (μM) |
| C-22 | A | C-13 | A |
| C-21 | A | C-7 | A |
| C-20 | A | C-6 | A |
| C-15 | B | C-12 | A |
| C-17 | A | C-9 | B |
| C-16 | A | C-14 | A |
| C-11 | B | C-8 | B |
| C-10 | B | C-5 | A |
| C-4 | A | C-2 | A |
| C-19 | A | C-3 | A |
| C-18 | A | C-1 | A |
| C-31 | A | C-27 | A |
| C-34 | A | C-90 | B |
| C-35 | A | C-92 | B |
| C-24 | C | C-41 | A |
| C-25 | A | C-93 | B |
| C-94 | B | C-95 | B |
| C-42 | B | C-43 | B |
| C-44 | C | C-46 | B |
| C-47 | C | C-49 | B |
| C-51 | B | C-54 | B |
| C-57 | A | C-58 | B |
| C-60 | A | C-124 | A |
| C-61 | A | C-62 | A |
| C-63 | A | C-65 | B |
| C-64 | A | C-66 | A |
| C-67 | B | C-68 | A |
| C-69 | B | C-70 | B |
| C-71 | A | C-72 | A |
| C-73 | A | C-74 | A |
| D-1 | A | D-3 | B |
| D-2 | B | D-4 | B |

A < 0.1 μM;
0.1 μM ≤ B < 1 μM;
1 μM ≤ C

### Example B-2. Additional Biochemical Assays

Cell Based Assay: 15-PGDH is highly expressed in resting human lung adenocarcinoma cells (A549) (Tong et al., 2006), and this cell line was used to assess 15-PGDH inhibition by MF-300Na in vitro.

In this assay, A549 cells are treated with interleukin (IL) 1β, which induces the expression of cycloxygenase-2 and the synthesis of PGE₂ (Tong et al., 2006). In the studies evaluating test articles, thirty thousand A549 cells were seeded in 100 μL F12K completed media and incubated for 24 hours at 37° C. with 5% CO₂ before being serum-starved for 24 hours. On the day of the experiment, buffer was changed to complete medium, and cells were incubated for 30 minutes with compounds prior to the addition of IL-1β (final concentration of 0.1-0.25 ng/mL) overnight at 37° C. with 5% CO₂. Each concentration was run in triplicate. In this assay, tool compounds increased PGE₂ in the supernatant, and a half maximal effective concentration (EC₅₀) was calculated for each compound. The PGE2 in the supernatant was detected and quantified using a Cisbio HTRF technology (Homogeneous Time-Resolved Fluorescence) kit (62P2APEG-62P2APEH) according to the manufacturer's recommendations, quantifying the fold induction of PGE2 of cells treated with IL-1b plus test article, versus treatment with IL-1b only. Data can be seen in Table 4.

TABLE 4
PGE2-HTRF over IL1-B

| | PGE2-HTRF | PGE2-HTRF | PGE2-HTRF | PGE2-HTRF |
|---|---|---|---|---|
| | over IL1-β: | over IL1-β: | over IL1-β: | over IL1-β: |
| Compd. | Fold induction | Fold induction | Fold induction | Fold induction |
| No. | at 1 μM | at 0.1 μM | at 0.01 μM | at 0.001 μM |
| C-29 | 4.88 | 6.4 | 4.08 | 3.78 |
| C-30 | 3.79 | 2.5 | 1.68 | 1.16 |
| C-19 | 4.5 | 3.61 | 1.81 | 1.11 |
| C-25 | 6.39 | 4.84 | 3.56 | 2.08 |
| C-26 | 3.45 | 1.8 | 1.31 | 1.05 |
| C-27 | 4.68 | 3.36 | 2.06 | 1.04 |
| C-28 | 3.61 | 2.06 | 0.76 | 1.04 |
| C-6 | 4.88 | 5.31 | 2.14 | 0.98 |
| C-57 | 4.86 | 4.94 | 4.94 | 4.07 |
| C-59 | 4.32 | 4.74 | 3.09 | 0.91 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (V), or a pharmaceutically acceptable salt or solvate thereof: 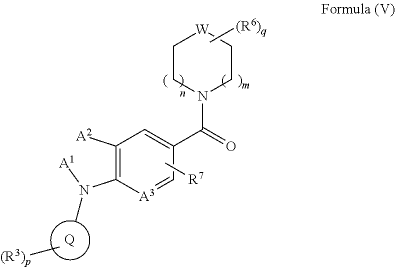
wherein,
ring Q is C₆ aryl or 5 to 10-membered heteroaryl;
A¹ is CR¹ or N and A² is CR² or N, provided that at least one of A¹ or A² is N;
A³ is N or CR⁷;
W is —CR⁶R⁶—, —O—, —S—, —NR⁵—, —S(O)—, —S(O)₂—, or —C(O)—;
R¹ and R² are each independently H, halogen, —CN, —OR¹⁰, —C(O)R¹⁰, —C(O)OR¹⁰, —NR⁸R⁹, —C(O)NR⁸R⁹, substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —OC(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁵ is H or C₁-C₆ alkyl; and
each R⁶ is independently H, halogen, —CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl;
or two R⁶ can join together with the atom(s) to which they are attached to form a C₃-C₆ cycloalkyl or C₃-C₈ heterocycloalkyl ring;
R⁷ is H, halogen, —OR¹⁰, —C(O)R¹⁰, —C(O)OR¹⁰, —CN, —C(O)NR⁸R⁹, —NR⁸C(O)R⁹, substituted or unsubstituted C₁-C₆ alkyl, or substituted or unsubstituted C₃-C₈ cycloalkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, and C₃-C₁₀ cycloalkyl;
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
n and m are each independently 0, 1, 2, or 3;
q is 0, 1, or 2; and
p is 1, 2, or 3.

2. The compound of claim 1, or a pharmaceutically acceptable salt or solvate thereof, wherein ring Q is a bicyclic or monocyclic heteroaryl comprising 1, 2, or 3 heteroatoms selected from O, S, and N.

3. The compound of claim 1, or a pharmaceutically acceptable salt or solvate thereof, wherein ring Q is a phenyl, pyrimidinyl, or pyridinyl.

4. The compound of any one of claims 1-3, wherein the compound has the structure of Formula (VI), or a pharmaceutically acceptable salt or solvate thereof: 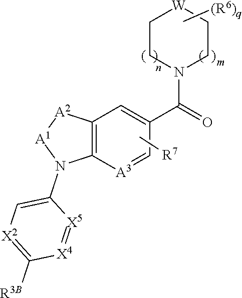
wherein,
X² is N, NR^{3A}, or CR^{3A};
X³ is N or CR^{3B};
X⁴ is N, NR^{3C}, or CR^{3C}; and
R^{3A}, R^{3B}, and R^{3C} are each independently H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)OR¹⁰, —NR¹²C(O)NR⁸R⁹, —NR¹²SO₂R¹⁰, —NR¹²SO₂NR⁸R⁹, —OC(O)NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R^{3A} and R^{3B} together with the atoms to which they are attached form a substituted or unsubstituted 5 to 6-membered aryl or heteroaryl; or
R^{3B} and R^{3C} together with the atoms to which they are attached form a substituted or unsubstituted 5 to 6-membered aryl or heteroaryl; and
wherein CR^{3A}, CR^{3B}, and CR^{3C} are not all H at the same time.

5. The compound of claim 4, wherein the compound has the structure of Formula (VIa), (VIb), or (VIc), or a pharmaceutically acceptable salt or solvate thereof:

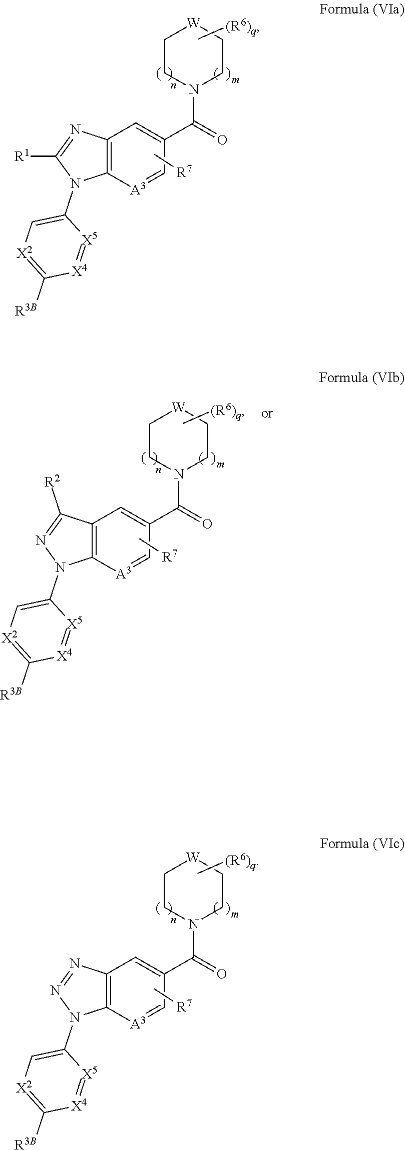

6. The compound of claim 4 or 5, or a pharmaceutically acceptable salt or solvate thereof, wherein X⁴ is CR^{3C} and X⁵ is CH.

7. The compound of claim 4 or 5, or a pharmaceutically acceptable salt or solvate thereof, wherein X⁴ is CR³C and X⁵ is N.

8. The compound of claim 4 or 5, or a pharmaceutically acceptable salt or solvate thereof, wherein X⁴ is N and X⁵ is CH.

9. The compound of any one of claims 4-8, or a pharmaceutically acceptable salt or solvate thereof, wherein R^{3B} and R^{3C} together with the atoms to which they are attached form a substituted or unsubstituted 5-membered heteroaryl.

10. The compound of claim 9, or a pharmaceutically acceptable salt or solvate thereof, wherein R^{3B} and R^{3C} together with the atoms to which they are attached form a 5-membered heteroaryl comprising 1, 2, or 3 N atoms.

11. The compound of claim 4, wherein the compound has the structure of Formula (VII), or a pharmaceutically acceptable salt or solvate thereof: 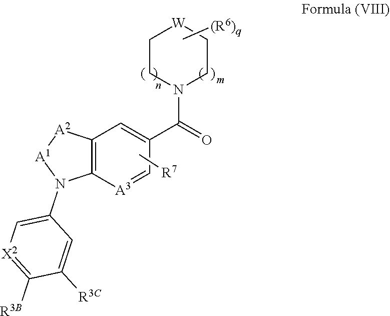
X² is N or CH; and
R^{3C} is H or halogen and R^{3B} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl; or
R^{3B} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

12. The compound of claim 11, wherein the compound has the structure of Formula (VIIa), (VIIb), or (VIIc), or a pharmaceutically acceptable salt or solvate thereof:

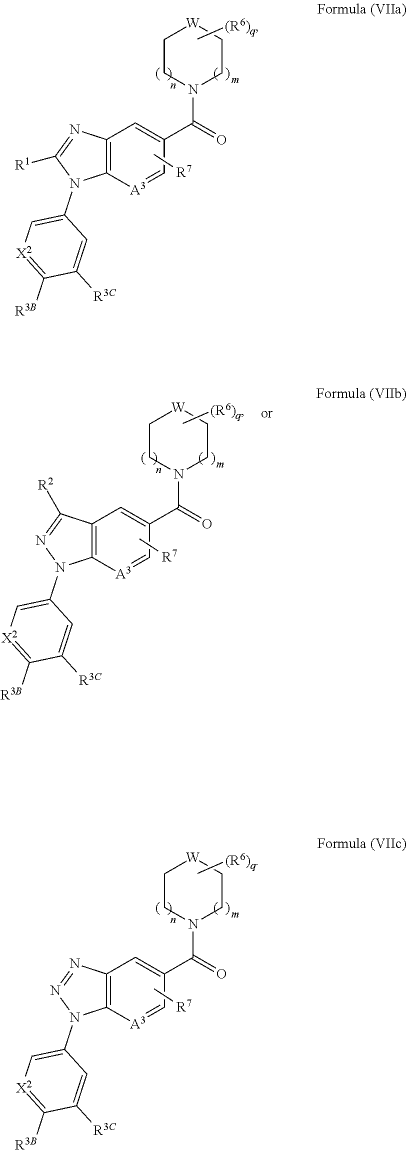

13. The compound of claim 11 or 12, or a pharmaceutically acceptable salt or solvate thereof, wherein R^{3C} is H or halogen; and R^{3B} is selected from H, halogen, —CN, —NR⁸R⁹, —OR¹⁰, —C(O)R¹⁰, —C(O)OR¹⁰, —C(O)NR⁸R⁹, —NR¹²C(O)NR⁸R⁹, —NR¹²C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

14. The compound of claim 13, or a pharmaceutically acceptable salt or solvate thereof, wherein R^{3C} is H; and R^{3B} is —C(O)R¹⁰, —C(O)NR⁸R⁹, —NR¹²C(O)OR¹⁰, substituted or unsubstituted C₃-C₈ heterocycloalkyl, or substituted or unsubstituted 5-membered heteroaryl.

15. The compound of claim 11 or 12, or a pharmaceutically acceptable salt or solvate thereof, wherein R^{3B} is a selected from H, halogen, —CN, —NR⁸R⁹, —OR¹⁰, —C(O)R¹⁰, —C(O)OR^{c}, —C(O)NR⁸R⁹, —NR¹²C(O)NR⁸R⁹, —NR¹²C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl; and R^{3C} is H or halogen.

16. The compound of claim 15, or a pharmaceutically acceptable salt or solvate thereof, wherein R^{3B} is —C(O)R¹⁰, —C(O)NR⁸R⁹, —NR¹²C(O)OR¹⁰, substituted or unsubstituted C₃-C₈ heterocycloalkyl, or substituted or unsubstituted 5-membered heteroaryl; and R^{3C} is H.

17. The compound of any one of claims 4-16, or a pharmaceutically acceptable salt or solvate thereof, wherein X² is N.

18. The compound of any one of claims 4-16, or a pharmaceutically acceptable salt or solvate thereof, wherein X² is CH.

19. The compound of claim 1 or 2, or a pharmaceutically acceptable salt or solvate thereof, wherein ring Q is a 5-membered heteroaryl selected from triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, and tetrazolyl.

20. The compound of any one of claims 1, 2, or 19, or a pharmaceutically acceptable salt or solvate thereof, wherein ring Q is 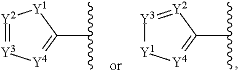
Y¹ is O, S, or NR^{3D};
Y² is N or CR^{3A};
Y³ and Y⁴ are each independently N or CR^{3B};
R^{3A} and R^{3B} are each independently selected from H, halogen, —CN, —NR⁸R⁹, —OR¹⁰, —C(O)R¹⁰, —C(O)OR¹⁰, —C(O)NR⁸R⁹, —NR⁸C(O)NR⁸R⁹, —NR⁸C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

21. The compound of claim 20, wherein the compound has the structure of Formula (VIIIa), (VIIIb), (VIIIc), (VIIId), (VIIIe), or (VIIIf), or a pharmaceutically acceptable salt or solvate thereof:

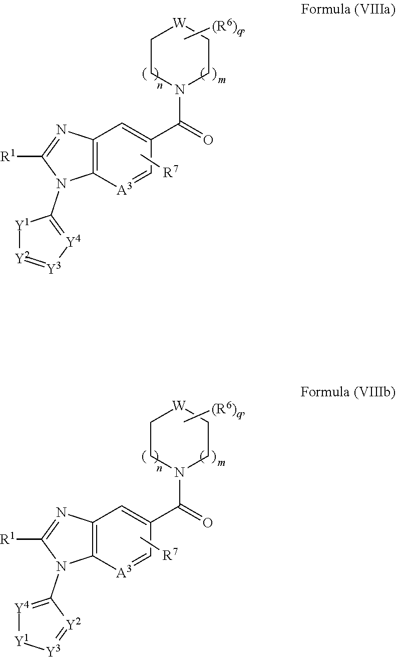

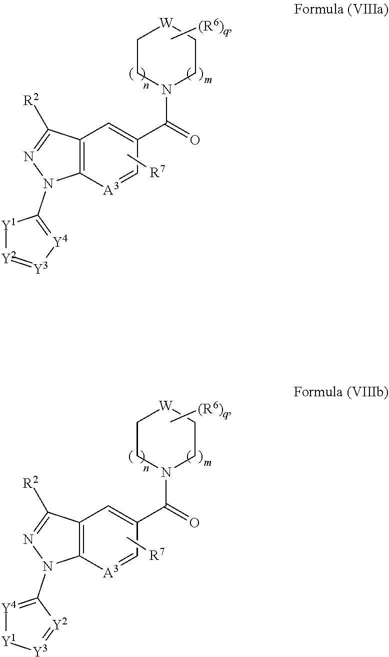

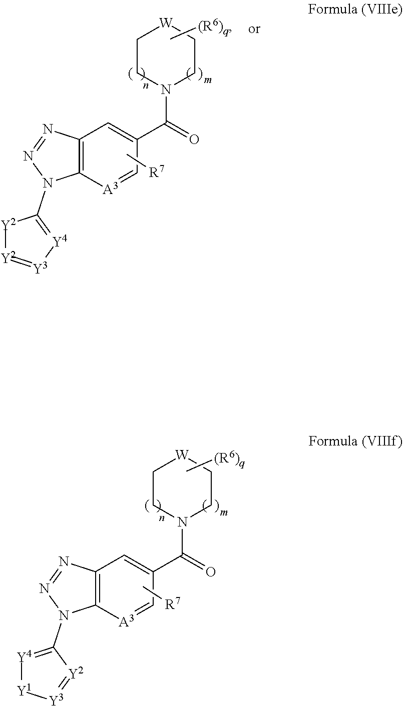

22. The compound of claim 20 or 21, or a pharmaceutically acceptable salt or solvate thereof, wherein:
Y¹ is O or S;
Y² is CR^{3A}; and
Y³ and Y⁴ are each independently N or CR^{3B}.

23. The compound of claim 20 or 21, or a pharmaceutically acceptable salt or solvate thereof, wherein:
Y¹ is O or S;
Y² is N; and
Y³ and Y⁴ are each independently N or CR^{3B}.

24. The compound of claim 1 or 2, wherein ring Q is a bicyclic heteroaryl comprising 1, 2, or 3 heteroatoms selected form N, S, and O.

25. The compound of any one of claims 1-24, or a pharmaceutically acceptable salt or solvate thereof, wherein W is —O—, —S—, or —S(O)₂—.

26. The compound of any one of claims 1-24, of a pharmaceutically acceptable salt or solvate thereof, wherein W is —NR¹³—.

27. The compound of any one of claims 1-24, of a pharmaceutically acceptable salt or solvate thereof, wherein W is —CR⁶R⁶—.

28. The compound of any one of claims 1-27, or a pharmaceutically acceptable salt or solvate thereof, wherein each R is independently halogen, —NR⁸R⁹, —OR¹⁰, —C(O)R¹⁰, —C(O)OR¹⁰, —C(O)NR⁸R⁹, or substituted or unsubstituted C₁-C₆ alkyl.

29. The compound of any one of claims 1-27, or a pharmaceutically acceptable salt or solvate thereof, wherein each R⁶ is independently F, —NH₂, —OH, —OCH₃, or —CH₃.

30. The compound of any one of claims 1-27, wherein R⁶ is not halogen.

31. The compound of any one of claims 1-27, or a pharmaceutically acceptable salt or solvate thereof, two R can join together with the atom(s) to which they are attached to form a C₃-C₆ cycloalkyl ring.

32. The compound of any one of claims 1-31, or a pharmaceutically acceptable salt or solvate thereof, wherein n is 0, 1, or 2; and m is 0 or 1.

33. The compound of any one of claims 1-27, or a pharmaceutically acceptable salt or solvate thereof, wherein: 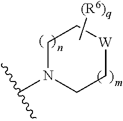
is

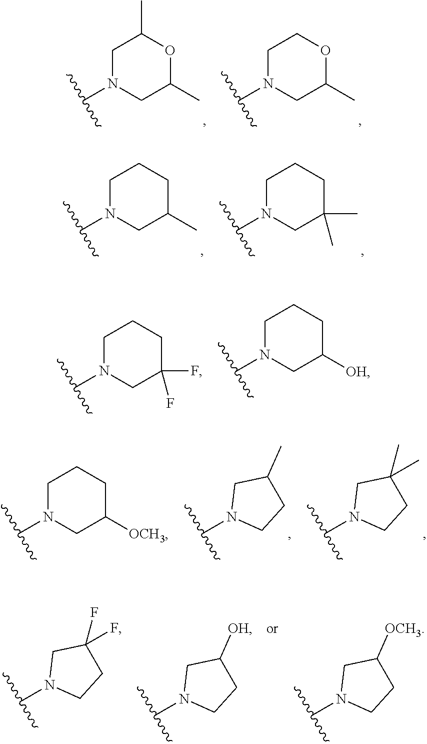

34. The compound of any one of claims 1-27, or a pharmaceutically acceptable salt or solvate thereof, wherein: 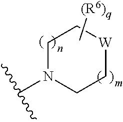
is

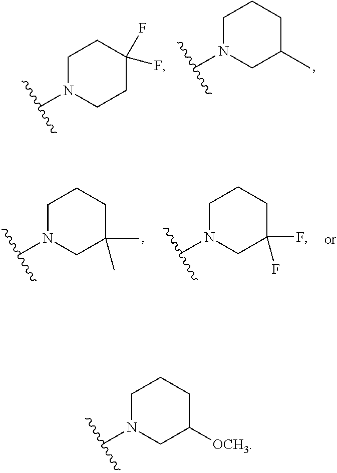

35. A compound of Formula (I), or a pharmaceutically acceptable salt or solvate thereof: 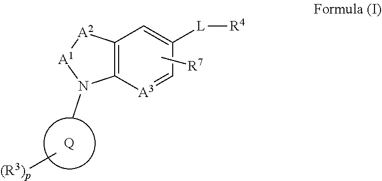
wherein,
ring Q is C₆ aryl or 5- to 10-membered heteroaryl;
L is —CR^{13A}R^{13B}, —C(O)—, —S—, —S(O)—, —S(O)₂—, or —S(O₂)NH—;
A¹ is N or CR¹ and A² is N or CR², provided that at least one of A¹ or A² is N;
A³ is N or CR⁷;
R¹ and R² are each independently H, halogen, —CN, —OR¹⁰, —C(O)R¹⁰, —C(O)OR¹⁰, —NR⁸R⁹, —C(O)NR⁸R⁹, —NR⁸C(O)R⁹ substituted or unsubstituted C₁-C₆ alkyl, or substituted or unsubstituted C₃-C₈ cycloalkyl;
each R³ is independently selected from H, halogen, —CN, —NR⁸R⁹, —OR¹⁰, CN, —C(O)R¹⁰, —C(O)OR¹⁰, —C(O)NR⁸R⁹, —SOR¹¹, —SO₂R¹¹, —SO₂NR⁸R⁹, —NR¹²C(O)R¹⁰, —NR¹²C(O)NR⁸R⁹, —NR¹²SO₂R¹⁰, —NR¹²SO₂NR⁸R⁹, substituted or unsubstituted C₁-C₆ alkyl, substituted or unsubstituted C₁-C₆ haloalkyl, substituted or unsubstituted C₃-C₈ cycloalkyl, substituted or unsubstituted C₃-C₈ heterocycloalkyl, substituted or unsubstituted C₆ aryl, or substituted or unsubstituted 5- to 10-membered heteroaryl;
R⁴ is substituted or unsubstituted C₁-C₈ alkyl, substituted or unsubstituted C₂-C₈ alkenyl, substituted or unsubstituted C₁-C₈ heteroalkyl;
or R⁴ is 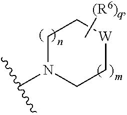
wherein
W is —CR⁶R⁶—, —O—, —S—, —NR⁵—, —S(O)₂—, or —C(O)—;
R⁵ is H or substituted or unsubstituted C₁-C₆ alkyl, substituted or unsubstituted cycloalkyl, or substituted or unsubstituted heterocycloalkyl;
each R⁶ is independently H, halogen, CN, —NR⁸R⁹, —OR¹⁰, —C(O)R¹⁰, —C(O)OR¹⁰, —C(O)NR⁸R⁹, —SOR¹¹, —SO₂R¹¹, substituted or unsubstituted C₁-C₆ alkyl, —NR⁸C(O)R⁹, —SR, or C₃-C₈ cycloalkyl;
or two R⁶ can join together with the atom(s) to which they are attached to form a C₃-C₆ cycloalkyl or C₃-C₈ heterocycloalkyl ring;
n and m are each independently 0, 1, 2, or 3; and
q is 0, 1, 2, 3, 4, 5, or 6;
R⁷ is H, halogen, —OR¹⁰, —C(O)R¹⁰, —C(O)OR¹⁰, —CN, —C(O)NR⁸R⁹, —NR⁸C(O)R⁹, or substituted or unsubstituted C₁-C₆ alkyl, or substituted or unsubstituted C₃-C₈ cycloalkyl;
each R⁸ and R⁹ are independently selected at each occurrence from H, C₁-C₆ alkyl, C₂-C₆ alkenyl, C₂-C₆ alkynyl, C₁-C₆ heteroalkyl, C₁-C₆ haloalkyl, C₃-C₁₀ cycloalkyl and C₃-C₁₀ heterocycloalkyl
each R¹⁰ is independently selected from H, C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹¹ is independently selected from C₁-C₆ alkyl, C₂-C₆ alkenyl, C₁-C₆ heteroalkyl, C₁-C₆ haloalkyl, C₃-C₈ cycloalkyl, C₆-C₁₀ aryl, and 5- to 10-membered heteroaryl;
each R¹² is independently selected from H, C₁-C₆ alkyl, C₂-C₆ alkenyl, C₁-C₆ haloalkyl, and C₃-C₈ cycloalkyl;
R^{13A} and R^{13B} are each independently H, CF₃, halogen, or C₁-C₆ alkyl; and
p is 1, 2, 3, or 4.

36. The of claim 35, wherein the compound has the structure of Formula (IIa), (IIb), or (IIc), or a pharmaceutically acceptable salt or solvate thereof:

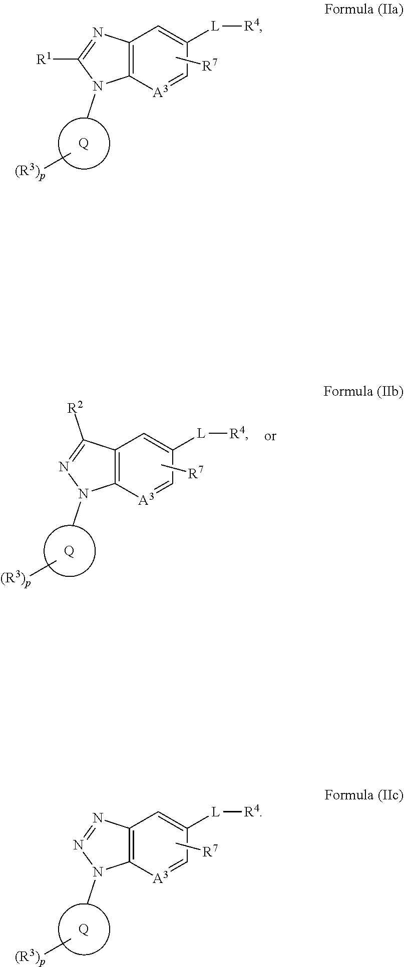

37. The compound of claim 35 or 36, or a pharmaceutically acceptable salt or solvate thereof,
wherein:
L is —S—, —S(O)—, or —S(O)₂—; and
R⁴ is substituted or unsubstituted C₁-C₈ alkyl.

38. The compound of claim 35 or 36, or a pharmaceutically acceptable salt or solvate thereof, wherein:
L is —C(O)—; and
R⁴ is

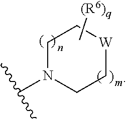

39. The compound of any one of claims 35-38, or a pharmaceutically acceptable salt or solvate thereof, wherein q is 1 or 2.

40. The compound of any one of claims 35-39, or a pharmaceutically acceptable salt or solvate thereof, wherein ring Q is phenyl or a 6-membered heteroaryl.

41. The compound of any one of claims 35-39, or a pharmaceutically acceptable salt or solvate thereof, wherein ring Q is phenyl, pyrimidinyl, or pyridinyl.

42. The compound of any one of claims 35-39, or a pharmaceutically acceptable salt or solvate thereof, wherein ring Q is 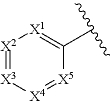
wherein;
X¹, X², X³, X⁴, and X⁵ are each independently N or CR³; and
wherein at least two of X¹—X⁵ is CR³.

43. The compound of claim 42, or a pharmaceutically acceptable salt or solvate thereof, wherein:
X¹ and X⁵ are each independently N or CH;
X² is N or CR^{3A};
X³ is N or CR^{3B};
X⁴ is N, NR^{3C}, or CR^{3C}; and
R^{3A}, R^{3B}, and R^{3C} are each independently selected from H, halogen, —CN, —NR⁸R⁹, —OR¹⁰, —C(O)R¹⁰, —C(O)OR¹⁰, —C(O)NR⁸R⁹, —NR⁸C(O)NR⁸R⁹, —NR⁸C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

44. The compound of any one of claims 35-39, or a pharmaceutically acceptable salt or solvate thereof, wherein ring Q is a 5-membered heteroaryl.

45. The compound of any one of claims 35-39, or a pharmaceutically acceptable salt or solvate thereof, wherein ring Q is a 5-membered heteroaryl selected from triazinyl, pyrrolyl, furanyl, imidazolyl, pyrazolyl, oxazolyl, isoxazolyl, thiophenyl, thiazolyl, isothiazolyl, triazolyl, oxadiazolyl, thiadiazolyl, and tetrazolyl.

46. The compound of any one of claims 35-39, or a pharmaceutically acceptable salt or solvate thereof, wherein ring Q is 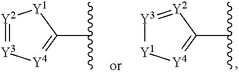
Y¹ is O, S, or NR^{3D};
Y² is N or CR^{3A};
Y³ and Y⁴ are each independently N or CR^{3B};
R^{3A} and R^{3B} are each independently selected from H, halogen, —CN, —NR⁸R⁹, —OR¹⁰, —C(O)R¹⁰, —C(O)OR¹⁰, —C(O)NR⁸R⁹, —NR⁸C(O)NR⁸R⁹, —NR⁸C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

47. The compound of any one of claims 1-46, or a pharmaceutically acceptable salt or solvate thereof, wherein each R³ is independently selected from H, halogen, —CN, —NR⁸R⁹, —OR¹⁰, —C(O)R¹⁰, —C(O)OR¹⁰, —C(O)NR⁸R⁹, —NR⁸C(O)NR⁸R⁹, —NR⁸C(O)OR¹⁰, —OC(O)NR⁸R⁹, substituted or unsubstituted C₁-C₆ haloalkyl, substituted or unsubstituted C₃-C₈ cycloalkyl, substituted or unsubstituted C₃-C₈ heterocycloalkyl, and substituted or unsubstituted 5-membered heteroaryl.

48. The compound of claim 47, or a pharmaceutically acceptable salt or solvate thereof, wherein each R³ is independently selected from H, halogen, —C(O)R¹⁰, —C(O)NR⁸R⁹, —NR⁸C(O)NR⁸R⁹, —NR⁸C(O)OR¹⁰, substituted or unsubstituted C₃-C₈ heterocycloalkyl, or substituted or unsubstituted 5-membered heteroaryl.

49. The compound of any one of claims 1-47, or a pharmaceutically acceptable salt or solvate thereof, wherein each R³ is independently selected from the group consisting of

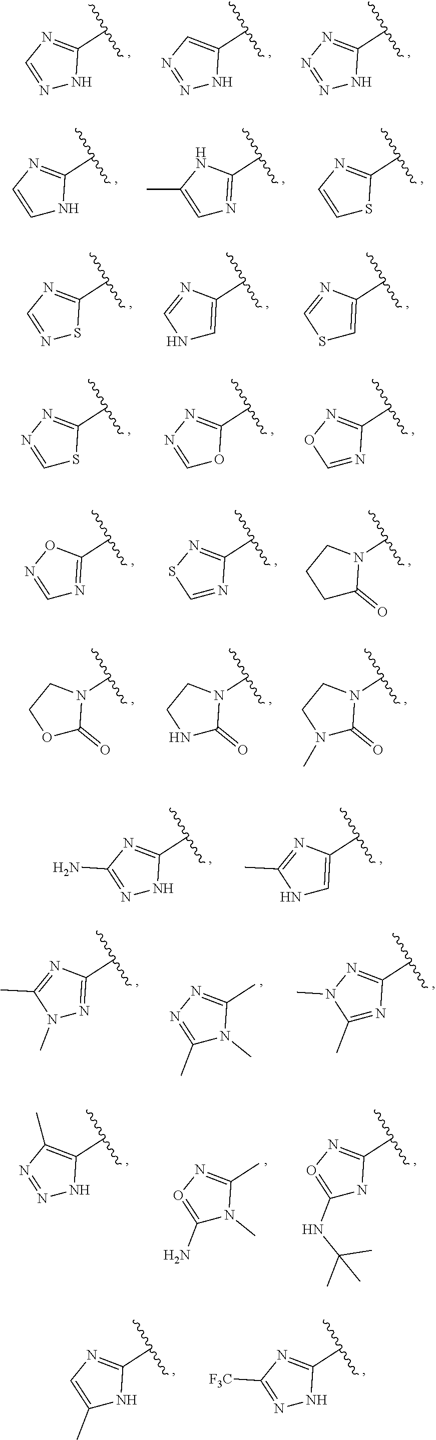

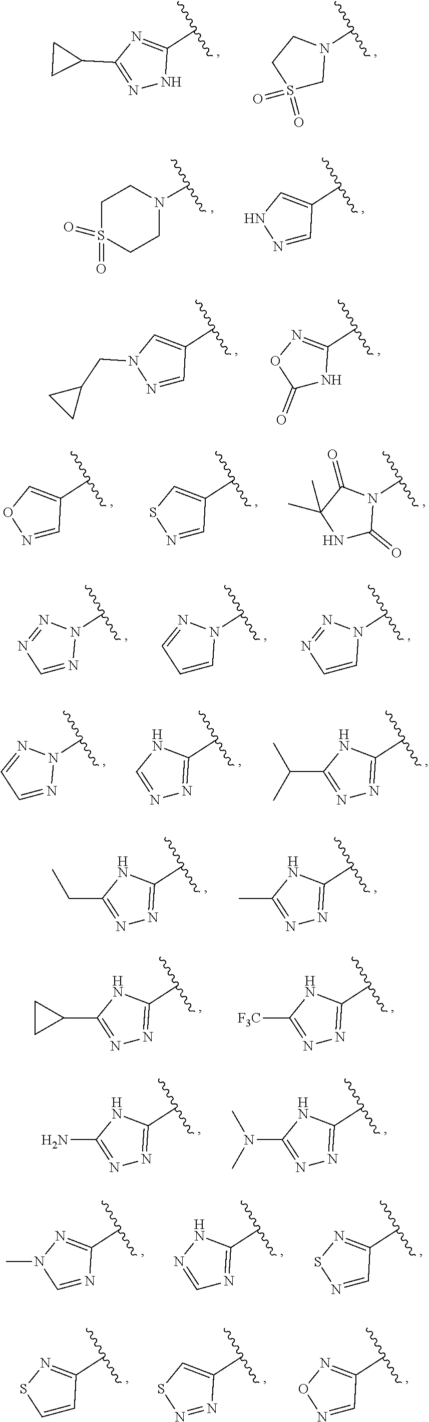

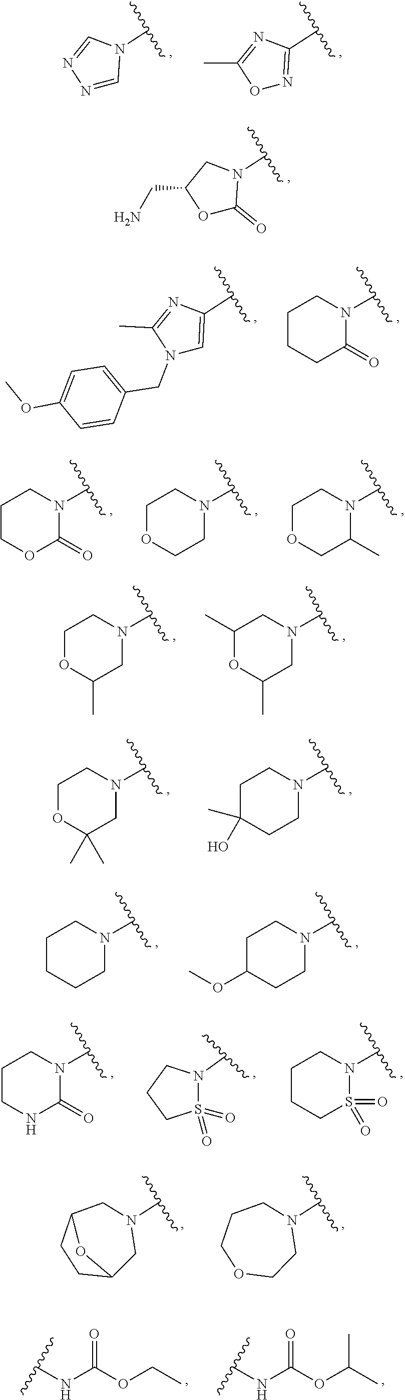

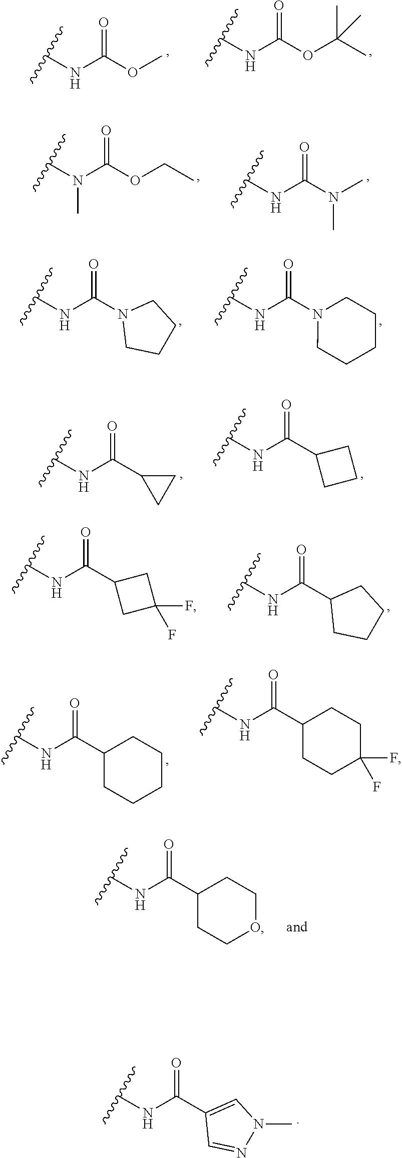

50. The compound of any one of claims 1-49, or a pharmaceutically acceptable salt or solvate thereof, wherein:
R¹ is H or substituted or unsubstituted C₁-C₆ alkyl; and
R² is H or substituted or unsubstituted C₁-C₆ alkyl.

51. The compound of any one of claims 1-49, or a pharmaceutically acceptable salt or solvate thereof, wherein R is H and R² is H.

52. The compound of any one of claims 1-51, or a pharmaceutically acceptable salt or solvate thereof, wherein A³ is N.

53. The compound of any one of claims 1-51, or a pharmaceutically acceptable salt or solvate thereof, wherein A³ is CR⁷.

54. The compound of any one of claims 1-53, or a pharmaceutically acceptable salt or solvate thereof, wherein R⁷ is H.

55. The compound of any one of claims 1-54, wherein the compound is selected from Table 1a or Table 1b, or a pharmaceutically acceptable salt or solvate thereof.

56. A pharmaceutical composition comprising a compound of any one of claims 1-55, or a pharmaceutically acceptable salt or solvate thereof, and a pharmaceutically acceptable excipient.

57. A method of promoting and/or stimulation skin pigmentation, comprising administering one or more of said compositions of any of the preceding claims to a subject in need thereof.

58. A method of inhibiting hair loss, comprising administering one or more of said compositions of any of the preceding claims to a subject in need thereof.

59. A method of preventing and/or treating skin inflammation and/or damage, comprising administering one or more of said compositions of any of the preceding claims to a subject in need thereof.

60. A method of preventing and/or treating vascular insufficiency, comprising administering one or more of said compositions of any of the preceding claims to a subject in need thereof.

61. A method of preventing, treating, minimizing and/or reversing congestive heart failure, cardiomyopathy, comprising administering one or more of said compositions of any of the preceding claims to a subject in need thereof.

62. A method of reducing cardiac ejection fraction, comprising administering one or more of said compositions of any of the preceding claims to a subject in need thereof.

63. A method of preventing and/or treating a gastrointestinal disease, comprising administering one or more of said compositions of any of the preceding claims to a subject in need thereof.

64. A method of preventing and/or treating renal dysfunction, comprising administering one or more of said compositions of any of the preceding claims to a subject in need thereof.

65. A method of stimulation bone resorption and bone formation, comprising administering one or more of said compositions of any of the preceding claims to a subject in need thereof.

66. A method of stimulating tissue regeneration by stimulating, comprising administering one or more of said compositions of any of the preceding claims to a subject in need thereof.

67. A method of modulating cervical ripening, comprising administering one or more of said compositions of any of the preceding claims to a subject in need thereof.

68. A method of promoting neuroprotection and/or stimulating neuronal regeneration, comprising administering one or more of said compositions of any of the preceding claims to a subject in need thereof.

69. A method of treating and/or preventing a neurological disorder, a neuropsychiatric disorder, a neural injury, a neural toxicity disorder, a neuropathic pain, or a neural degenerative disorder, comprising administering one or more of said compositions of any of the preceding claims to a subject in need thereof.

70. A method of treating and/or preventing fibrotic or adhesion disease, disorder or condition, comprising administering one or more of said compositions of any of the preceding claims to a subject in need thereof.

71. A method of reducing and/or preventing scar formation, comprising administering one or more of said compositions of any of the preceding claims to a subject in need thereof.

72. A method of treating and/or preventing muscle disorder, muscle injury and/or muscle atrophy, comprising administering one or more of said compositions of any of the preceding claims to a subject in need thereof.

73. A method of treating and/or preventing fibrosis, comprising administering one or more of said compositions of any of the preceding claims to a subject in need thereof.

74. A method of treating and/or preventing idiopathic pulmonary fibrosis, comprising administering one or more of said compositions of any of the preceding claims to a subject in need thereof.

75. A method of treating and/or preventing kidney fibrosis, comprising administering one or more of said compositions of any of the preceding claims to a subject in need thereof.

76. A method of stimulating muscle regeneration, comprising administering one or more of said compositions of any of the preceding claims to a subject in need thereof.

77. A method of promoting organ fitness, comprising administering one or more of said compositions of any of the preceding claims to a subject in need thereof.

78. A method of promoting wound healing, comprising administering one or more of said compositions of any of the preceding claims to a subject in need thereof.

79. A method of treating acute kidney injury, comprising administering one or more of said compositions of any of the preceding claims to a subject in need thereof.

80. A method of treating sarcopenia, comprising administering one or more of said compositions of any of the preceding claims to a subject in need thereof.

81. A method of treating a neuromuscular disease, comprising administering one or more of said compositions of any of the preceding claims to a subject in need thereof.
